SEQUENCE LISTING (17 sequences)

SEQ ID NO: 1 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - protein, 190 aa, Homo sapiens
MGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 6 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 7 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 8 - protein, 209 aa, Homo sapiens
MGSVSNQQFAGGCAKAAEEAPEEAPEDAARAADEPQLLHGAGICKWFNVRMGFGFLSMTARAGVALDPPVDVFVHQSKLHMEGFRSLKEGEAVEFTFKKSAKGLESIRVTGPGGVFCIGSERRPKGKSMQKRRSKGDRCYNCGGLDHHAKECKLPPQPKKCHFCQSISHMVASCPLKAQQGPSAQGKPTYFREEEEEIHSPTLLPEAQN

SEQ ID NO: 9 - DNA, 122775 bp, Homo sapiens
tgagaatgatccatgtgttgcagaaaagaatgtctattctgcaggcatcggatgaaatgttctgtaaatatctatgaggctcattctacagtgcagattaagtctgatgtttctttgttgattttctgtctagaatatctgtccaaagctgaaagtgggatgttgaagtctccagctgttattgtattgaggtctagcagtctctttagctctaataatatttgccttatatgtctagatgctcaagggtttggtgcatatacatttacaattgttagatcctcttactgaattgagccttttctcattatatgatgacctttgtctcttctgatagtttttgttttgaagtctgttttgtctgatataaatatagctactccggctctttttttggtttccattggcttggaatatctttttatgttccttattttcagtctatttgtgtcattataggtcacatatgtttattgtaggcaatagaccaatgggtcttattttttaatccattcgcccactctatgtcttttgattgcagagtttagtccatttaattcaatattattattgataagtaaggacttactcctgctctttatttgctttttggttgttttggggtcttctcttccttctttcatttctccctgtctgccttttagcaaaggtaattttctctggtggtatgatttaatagcttgctttttatttttgtgtattcattgtgtttctttgacttgaagttctcgtgagccttgcaaatactatcttcttataacccactattttatgttagtaattacttaacactgattacataaacaaagaaaactaataagagctctacaccttaacttcctccccttgcttctaaactctttgttgtttctccttatattttattgtgctgtctatgtcttgacaagttgtcgtagttattatttttgattggttcatcatttagtctttctacttaagataagagtagtttacacaccacagttacagtgttataacattcggtgtttttctgtgtacttacgattaccagggagttttatatcttcagatgatttcttattgcttgttaacatccttttctttcagattgaataactccttgtagcatttcttgtaggacaggtctggtgttgatgaaatccctctgcttttgtttgtctgagaaagtctttatttctccttcatgtttgaagaatatttttgctgtatatattattctagggtaaaagttttttccctccagcagcttaagtatgtcacgccactctctcctgggctgtcaggtttccactgaatagtctgctgccagatgtattgcagctccattgtttgttaattctttcttttcttttgctgcttttaggatcctttatcctagacctctgggaatttgattattgcatgccttggagtagtcatctttgggtttatctgctaggtgttctatacaattcttgtatttggatattgatacttttctataggtttgagtggttctctgatattatccctttgaataatctttctaccccatctctttctcgaccttgtctttaaggccagtaactcttagatttgcttttttaggtaattttctagatcttgtaggcatgcttcattattttttattcttttttctttgtcttctctgactgtattttctaatagcctgtctttaaaagtgcactatttcttctccttaatcaattctgctattgaaagactctgatgcattcttcagtatgtcagttgcatttttaaacttgagaatttctgcttgattctttaaaattatttcaatatctctattaaatttatctgatagaattctgaattctttttctgtgttatcttaattttctctgagtttcttcaagatattgattttgaattctctgtctgaaacatcacatatctctatttctccagggttgttccctggggacttatttagtttgtttggtgaggtcatgttttcctgggtggtctggaagcttttggatattcattggtgtctgggcattgaagaccaccccaagagcctgcttagtactctacctccctgtggctgagctggtacctaaggggaaggaaaacatcctctttacttttccttctgcttttgtcaagcagaaggagtctctcacattagccaccacagttgggaatatactgggtctcacctgaaaccagcagatctcagagtcacacccaagaaccacagtgtactacctgagtattgcttctggctattgagaacccaagggctctttagccaacaggtgatgggtcctgcccagactgtttctttcccttcaaggcagcagtttcccttctggcccaggatgtgtctagaaatgttatccacaagttaggacctgggatgggggtctgatgactcagactgatgccctgtcctactgtggctaaactgatattcaagataacaaagtcctctttactcttccccctcttctcttcaagcagaaggaatgggtctcttcgtgctgtttttaatcactccagccactagcattgggactgatgcagacactaaggatccaaatgattatttttgttttgtttttttaactgcattgggattaggcaacagaagggtctaatgtggctgagagtttttgagagaggagcatttttaggagggtagctgcattccaagtagagagctctgctggggtaaaagaggcatgagtgctagaaaagggcatgcagtgatgaagagccactaacagcactacctggggttggggtaggggtgtcacaagcactcccttagctcccctgtctggtgtctcagtaagtcgcatgccctccaagtctactggctctgagctcagcatttgggcttgcttaggagttgctgtccttgtggcctaggctgcctttaaggttatttggggccccagagcactttactgaaactcaagttttcaccactggtatgggctatttcctctgagggaaattggctgagttcagcctagttttgctttctgctatgacagagcagtactgagttcaattcaaagtctcccaatcactgcactctccctttcccaagccacaaggctgctgctgctgggggatggaggactggcggcattggcaaatcaaaactgtctttcctggcttcttcagtgcctctttcagtaatatgaaattaaaaccaggtattgtggttactcacctgatttttggttcttataaaggtgctttttgtgtgtgtagatagcagttaaattgtgttcctagccggggtggggggtgggtggtcaaaccttctatttgaccatcttgcttcacccctttctactgatactattgttgttgatattattaggaatgaaaatgagccctgtgagttataattgtcttcattttgcagaccaggaaactgaggctcagagaaatattaagcagccaaagtcacagcgattaaaagaggcgggaccaaactttgacggagatcaggacccactcctttgagaaatcacctcttttgctagctccagcctaaaagtgctatctaggagaaggtttatgggaccccagcagatgttcaaccagaaacttctgacctgcaggggctgcagaggggaaatctgccctgattagaagagatttgttttccacccatattagtttgatctcacattgctataaagaactacctgagactgggtttcagaggctgtaccgtaggcatggccagagaggcttcaggaaacttacaattatggtggaagaagggaaagcaagcacatcttcacatggcagaaggagagacagagcgagcaaaggggaagtgccacacacttttaaatcatcagatatcctgagaactcactcactatcatgagaacagcaagggagaaatccgccctcataatccaatcacctcccactaggtccctctcccagtgaattataattcaatatgagatttggatggggacacagagccaaaccatctaaccttgccatgtcctactgtactttttggaagttttctgaactaacatttcttgtctagaaatatacacactcaagatcaatctaactattaaaaacaaaaaatgcttattttccagtacctgttttatgttagattctggggtaaaaagaaaaaggtactgacttaatgttcctgtctatcattcatttgtagcttgtgcatccatcctttcatctgtctatccatatcacaaacatgtattaaacacctgctgtatgctagacaccatcctgaaggtgcatttcttccctaagtcttttttttgcctgccggagagtacagtattctcaattcaagataagaagtggtgaataagagtgaagtgcaaattggtgaggacacttagacaccgagtgattacttctgacaggtttccaccactcagctaagtagcaaggtgaagactccgaggttgagttgtaactcacagagcgggaagaccaataggcagtgagaaaagaagttgcagtggcttcacgttatagcagatgcctgtgtgtacacatgggagcgaatatggctttatgctgtttttcaaccgcttcagccactagcattggtactgctgcacactaaggatccaaatgattgtttttttgttttttgttttgttttgttttgttttgttttgttttaaattgcattgggattaggcaacagaagggtctaatgcggccgggatgagacaggagagtttttaggagggtagctgcattctaagtaagggactctgcggggggaaaagaggcgcaagcgttgcaagaagggagtgcagggggttgagcaggcacctctacaggaaatggatgctgtccaggtgctggtgggcgccccagggctacgtggcgaagcagctcagccggtccaatcagagtgcgtccagggctcgggtttcgcgatctttaagtgactgaggcagatccccacgcggcacctggccatgctctcagctctcccgccgcggtgagtatgtttaaggaatcctttccattacggcggccccatacctaggtccccgtccgggacagaggaagccgcaacgggtccccccgggcacccgggcctttctcctgcctcccgccacgtccgagggtccggccgcagcgccgcctgagcccctccgcggccggcagtgggaggcgggctctccgaaagccgtcgcggtggtcccagaagccgggtcataaataattcacgagccagggtctggcgagctaagggaagggcggcggcagcggtgactagggcgggagcaggagcgggagccgggtgcacggtgagtagctgggtcctcatcccggagcgagagaggcatctgctgaccaggcgcggggctgagcgcactccttccactgtactgggggtgtacagtgaggagtggacgggttcgctctgcgccccccttaccctagccatctgcgccgcctccctggcccctcagcaggtggtgcgggcccggacagcggctgggggccagaggaactgcggggagagcgtggtggtgccggtgcccgtccaacccaaactttttagtctgagtggtggcccgcgacgccccgcttgggtcttctttccatccactggggatgggggcgggggtgggggtagccggcggccgtggttccggagcgggagtcggaggtgagtgtgcgaactggaccggggccccagagacttcggactcgctctcggggagaacggtgaccgaggtgagactcgccagggtcttgggaacgagtctgcggtaaaggaaactctagggatggccccggggacgtgaggcgctgagcttccctggctgagtgcggaaatgggccaacactgtgcagccgaccacagcgggagtagggatatgggggtacaaagcgaggccatctccccgcgtccgtgggagaaagcatgcacccctggcttgaagcaccttcccagcctctcgctccacggcccttctgcttgcatgcgcggggacccgcggctatcgctagatggagaactggggtgctcaaggcgcaccctgccagaacacgccgaccggacaacaaagaggagcgtggacagaccaggagcttcacggctgtagaaagtcagaggctcgggatcagtttccccgaaagcccgtgtctggaggggcagggagtcggatcggagggaggaactagctccagaaccccttctctctggcaagatcgcccccgatcggtgcccaacaggggccgcggcggggaactggcgccgacgcgctcagagcagccaagttactccggcaaccccctcccctgcgcggctgcgaattcctggagtctcttggcttcccgcgcccgccccgccgctcattcttttgtgtcccgcagctcgcgagaggcttccgcccagaagggagctaggcctggagggggcttggcctggaggtggtttggaggtaaagggcggggaagccggcgccccaggcttcattctgcaacaggttgctcctggagcccgctaaggcgactgacctttctggagaaaggtttgctggagttcctgagggcggcagctgttctttgttaagaaagcaagtgcacgatcgtagttcttgagtgccccccgagactcatgcgacacgaacaaccgctgcgatatgttgggttgaatcataagaaattgtcggttttttggatggaagatgtgaatatcgtcaacctcatatgattcaacttgtgtgatgttactcatttttcttttctcaccgtcctttctgtcccggttatcctaggagccccggtggagttgagctcagagtattgatgctagcagaagcagacagaccccagatagccttccagctgcgtgccaggtgctttacctattagatcgctagcctgcgaaccctatgcgaaatcctcctttgacagaggagcagaagaagggttggagaattcaaacaactgtcatcttggcagagttagtaagggggaccagagcccccttactgttgctgttgtctgtctattcagcagtgccctttagtttattcttgttttttttttcatgtgttccataatttttttctaaacttccttctgatttctaaacttttctaaacttctttctgatttctaaacttccttctgatttttatacgagacagtttctttgtgttttagtacaatgaggtgaacaaatgcatcagaggacaagctgaaaactttaaaccaagttggtactgtttaaaatataaaggaaataggtttttcagggagcaaagagctatttctgagatttgttaagggtcaagatactttgttaaaacagtggaagctgagtcgggggtaatttttatcagataaaggacttcttggattctgtaagttgcctgttcactctgatggtagtttcttttgctctgcagaagctctttagtttaattagatcccatttgtcaattttggcttttgttgccattgcttttggtgttttagacatgaagtccttgcccatgcctatgtcctgaatggtattgcctaggttttcttctagggtttttatggttttaggtctaacatttaagtttttaatccatcttgaattaatttttgtataaggtgtaaggaagggatccagtttcagctttctacatatggctagccaggtttcccagcaccgtttgttgggaattgaacaatgaaaacacttggacacaggaaggggaacatcacacaccggggcctgttgtcaggtggcgggagtggggagggatagcattaggacatatacctaatgtaaatgacgagttaataggtgcagcacaccgacatggcacatgtatacacatgtaacaaacatgcacattgtgcacatgtaccctagaacttaaagtataataaaaaaatatatatacatatacatatatatatgtatatgtatatatataaaaggacttcttgaaaggatggtccctgggtggcttaggtaagtggcagcaaggctggctagccaggcttaggaatctaggacatgccacaggacccctgatgggtgggtggctgcggctgccagaacaggaccctggatgtggctactggcattgctgccatcattgctggaatgaattagatactgtcctgcttcagagtgcgcaggcccaggcccagaagaaagcttctgattggcccagcatggctcagctccaccaggacacagtttgggcgcctcagctgacctcacaatgcaaggcagggctgcctcccacccagctcacaaacgtggtctcagatgttgggtatcccccatgcccccgccaaaagcactccagatgacaaaaatccactacaaagctcttctttaaaaagaaagattctgagaagttccatcaaaagaagaaaagaagaaatgaaggtgattgatcaccatatttgctttttcatttttaatttttttttttgagatggagtctctgtcacccaggctggagtgcagtggcatgatctcaggtcactgcaacctccgcctcccgggttcaagtgattctcctgcctcagcctcccgagtagctgggactacaggtgtgcaccaccacacctggctaattttttttgtatttttagtagagacagggtttcaccatgttagccaggatggtctcgatctcctgacctcgtgatccgcccgcctcggcctcccaaaatgctgggattacaggcttgagccaccacgcccgggccatatttgcttttaaaccagtattgaaatgcagttctctggactccaggctcaaaatcatgtaaggtcgcccaggctggagtgcagtggtatgattatggctcactacaggctcagcttcctggactcaagtaatcctcctgcttcagacttccgagtagctgggactacagttgtgcaccactacgtttgctaatttaaattttttttttttttttttttttttttgtagagacagggcctctctatgttgcccaggctggtagcaattcttcattgagtaccaactgtgttccaggctctggggatacagaaataagccagggacctgcacagcagctggggtagatcaattcaaagcaatatgaaaagggcatttggggagctttcaccttacatgtgttattttccaatgtgtttgggaagcctgaagtgtccttggaaagtgacatgaattgactaggcgggtactccaggatcagagtactccaggaacagaactgttctcaaactttgtttgaaggatcagtccccaggaaagcttgttaaaaatgcagattaatgggtttcatcccagaaattaggtccattaatagtctgcatttcaactaaacaccccattttatcccgagacagatgctactctcagtggctctcggacctagtggggtttagctggtaaatactcagagcttctgtttttggcacaacctattaaacccgagataaaaaactcttcctctttgacttgactttaatcagtttctagtatttcatagttagtgagatattaatactttcttaaaatgtactttaaaacttcagagggacctacattttggagcaagatgtaaagaaatctggaagtgcaactgcagttgctgtctatctcaagtcattgtctcccatctttgctgcggtgagcagttatgtttcacttcactgggaatttcccttcccctgctttgtcactgagggagttggagagcctgtttgttcatagttcttcccttggcagtttctctctgtcctgggctgaggtttggtggaattaccagaaagtgggccttctcagcatctgagcctttaatccttccagctgctgtgctggaggcttagctctctgcctgtcctttagcaaaaaaaaaaaccaagataaagtgtacatgtggtgtgttgtgcttgcagtagatgtttgaagggagccctttggaaacaccagggtcaaggtgtctattgtggatgtgacggtgggcgtaacctgaagcgtgggcctgaggttagtactggcagccacttgggatctgagcagcccatgatttgacaaaggaagaatttggggcccttgtattggtagctttgtgcaggagtaattggagtgggacaggacttagaagatgaggcaatggagggattagatatgaattaacatctttgttcaaatagctcaagaaatcccatctgttgtccaaagctttctttgacttggaatattggtagtgggtcctctggcttcttgcaaagtatgtcaccttcttcatcatcccccttccctttcttacagtatgtgtttcaggagcaccctgtacttatcttgaaagttagtaagtatgtatctagctcacaaagtgacagcagcttgctcttgctccaggacacttaatacagagaggaagagtggtacacttgggatccttccaggttctgtgctgatttgcctagtctcatgaaaccgcacaaggaaataacatattttggcttctacgccagcacttgccctgtagctctggagcaggctgtgtttggtttccacccacctacctggattaggctgaaggccctgctctaaagctttgcagcctcctgtctggggcacttcttgctgcaggcaggttacatgcattggtgactctgctgctatttctttccctgccagggatggtgccatgagtgaaacaagctgctcagcaggcacttactaaacaccgactacgtgccatgcccagtgttaaggtctggggattgaaagaagagtaggacagagtccatcttggaggcactttctgtcccattcccttcccagatgctggagccacagctgcttttaggattcttgtgtctttctccgtctcccaaggggtaggtaagagtctccctctgccctggctttcatctctgagacccctagttgtagaggtggtgacttagggtgaagatttccatcaggattacttggtaagcttattagaaagacaggttccagtgttgcacatgtgtccagcctcaaacggtgtggtccctagacttgaggccatgcccttctactttggattcggggtgaggtggagagggggatgtttgaaatgagattagtgagtaaataagtagaatataaggcagagtaagatatatctccagcggcaaagagaacaacacacacttggagaagaaataagcatataagcatgtactggattgctggggccccagtggctcgtactagagcctgagagggagggcactgaagggacagtgtcttgatgtcatagttagatcttggactttgaaggcaaactagtgggtttgactcttggccgcatggttaattgaaggggtgggttgcccctccacacctgtgggtgtttctcgttaggtggaacgagagacttggaaaagaaaaagacacagagacaaagtatagagaaagaaataagggggcccaggggaccagcgttcagcgtacggaggatccatcctggcctctgagttcccttagtatttattgatcattcttgggtgtttctcagagagggggatgtggcagggtcacaggataatagtggagagaaggtcagcagataaacacgtgaacaaaggtttctgcatcatagacaaggtaaagaattaagtgctgtgctttagatatgcatacccataaacatctcaatgccttacagagcagcattgttgcccgcatgtcccacctccagccctaaggcggttttcccctatctcagtagatggaacatacaatcgggttttataccgagacattccattgcccagggacgggcaggagacagatgccttcctcttgtctcaactgcaagaggcatgccttcctcttatactaatgctcctcagcacagaccctttacgggtgtcgggctgggggacagtcaggtctttcccttcccacgaggccatatttcagactatcacatggggagaaaccttggacaatacctggctttcctaggcagaggtccctgcggtcttccggccttccgcaatgtttgtgtccctgggtagttgagattagggagtggtgatgactcttaacgagcatgctgccttcaagcatctgtttaacaaagcacatcttgcacagcccttaatccatttaaccgtgagtggacacagcacatgtttcagagagcacggggttgggggtaaggttatagattaacagcatctcaagctgaagaatttctcttagtacagaacaaaatggagtctcctatgtctacttctttctacacagacacagtaacaatctgatctctctttcttttccccacattagggagggcactgaagggacagtgtcttgatgtcatagttagatcttggactttgaaggcaaactagtgggtttgactcttggccacatggttaatcacctgtgaccttgggcaagtcaccagaatcctctctgtgtcagtttcctcatctgcaaaatggaataatgccttaaacaaggaggaccttgtttctatctatgaagcatggagaacagtgcctggaagatagtaagtgctcaataaatgttagtttccaagatctcaagctttctctacaatatttagtatttcccgaacccataaagggtctgtgttctgagtctggccctcctgagaagggaccttctgtgagtcatggctttgatgccagcacctgccctgtggctctggagcaggttgtgtgcttggtttccacccacccatccggattaggctggaggccttgctctaaagctttgcagcgtcctctccgggcagctccaggtgcaggcagtttacacgcattgatgactctgctgctgtttctctccctgccagggatggtgccttgagtgaatgacccccttggagaacattcttccgcatccctcgcctcaagccagcctcagacagaaaactgaagattcagcagatccagtgcttcctgctcctcttctgcccaggaacacgcttgccttccccaaggcttccagaagctctgaggcaggaggcaccaagttctacctcatgtttggaggatcttgctagctatggccctcgtactcggctccctgttgctgctggggctgtgcgggaactccttttcaggagggcagccttcatccacagatgctcctaaggcttggaattatgaattgcctgcaacaaattatgagacccaagactcccataaagctggacccattggcattctctttgaactagtgcatatctttctctatgtggtacagccgcgtgatttcccagaaggtaagtgctgatggcagggaaagataggatttatctccaaacgcacccacaatattgttttttaatgtcaatgcaatcaatatataatatattcagtattttagcgattgctttgcacagaagcagaaagcatttagacagaagttaagaagacttgtaccaggttacataggttgcgaggaatgttattataagaaatggaagcaaacgatgttgctatgttggttttccacttctttaccttttgttttgcttggttttgctttgtttgggttagagcaatgaattattctaaggcacttaccaaaggtaaaagttagagtaaagttagggtaaaaatgcaaattgtgtgtcatcaaaggagaaacgaataactagcaactgatggccacctcatcgcttaaaaatttaactctcttctgcccaccacaatatgcaaattttgacattgcttagggtaaggacaaacatgtttacttgatcagctgttttcttcatttctaagagcttgggtgtgaggaagaggattggcactttccagaactgtcatgccatagtgctgttattcatcacacacagtgctgtttgtcattttcgccttcttaatgtttgttgttttccatactcttctgaattcagggtatgtgttcatagaaaagggattatgaaagtaaaaatagcctgtgggaaatatctgctactagggtttggcacctaaatttttgattggcttaacagtaagtccatcctaagcctgttcttttagttacaaagatttgattacaatcattgattatagaattcgctgtaatctttcttatcattttccttagctgttcagctactgtttgtaagagtcctactggtgtgtgtacatgcctgtgtgtgagagagagggaggaagagagacaggatgtatttagtgaatatcatcacatgtttatattatggggagtttatatattttcattaaaatacatttgtattggttttttaacacttatttgtcttagggcaattaaaacagcttgaagtatagttgtcaatttttgctaatttaaatttcagttcaccaaaacaaatctctttgaatggttattggcattgaagagaatcctgaacgaatgctacttactgtttatgttgctgttaaaaatactaactttggaaattccaaagacagaagtagaaaattagagtttatttcttaacattggatgcaaattcactcttctgaatagacatctcttggcttatttggagacttaatatgtgttactgaataaaataacctactaacatttttatttaaaaaaactctttaatttttgtcggtacctagtaggtgtatatattcacggggtacaagagatattttgatacagacatgcaatgtgtaataatcacatcatataaaatggtgtatctatctcctcaaacatttattctttgtgttacaaacaatccaattactcttttagttatttcaaaatgtacaattacattattattgactatagtcatcctgttgtgctatcagatactaggtcttattcatccttttaaactagtttcctactaatgtttttagtctttgcttcttggttttgttttaatataaaaggcatagaattcagttgtcgaatatgaatttacaatcatttcctcaaaaagactggaaagcttcccgtgtctcattctaaggcatatcatgtaattagttaatcccataatctctttccatatgttgctctattttggtccctttagatttgattggaaggtttcctcatacttttttttttaataaggaaaagcaatgtattttaaggtcttcattctggtgatcaagttcgaagatgtcttttagcttacacaacttccaatgcattacaaggttttgaactgcaacaacattgttagaccgaaggagggaactgaagaattgtaatcctaatgtaatctgagggtattagcctagtttgtctcttgaccttcatcaatacttattaccttggagaaggaaattctcaactttcaacagtagaccagggcagaccagcattacctctttgggaggaaaaattattaggttgcttaagtaatataatattgagtttaacagatgaatcttggggtgttggttttcttccatcatcctatttgtattctttcacctagttactacatttcctagtggtcttaaagtgcccactgggtgctttgcaatgtagaggacacatataagataatcatgatttttccatttggggccaatgcatttcacaagtaccaagtcataaatagatgccctcataattgaagatgcaaaaaacaaaacagaaagagtgagagcatgtgcatgattgagtcctatatattgtttattagtttaataccttagtacattaaccaaacacaagacttcaggacaggtcgtctgtggagtaatatcttcatagaagctattctcccatagatatatcctttcatttgaaagcgtgcaactggccagtgggctcttcgtgcctgctggccagatacagccgatatatcaatttagaggaattgcagtggagaaagagtttaattcaagcagagccagctgaatgggaggctggagttttattactcaaagcagtctcccccaaaattcagggattggggtttttaagtataatttggtgggtaggggattagaaagtggggagtgctgattggtcgggtagaagatgaaatcatagggaatagaagctgtcctcttgcactgtcagttcctgggtgggggtcacaagaccaggtgagccagtttatctatctgggtggcatcagctgatgcctcaaaacacagggtctgcaaagtactctgcaaagtatctgcaaagtatctcaagcactgattttaggctttacaacagtgatgttgtccctaggagcaactgggaggtttagaactttgtgacctctagctgtataactcctaaaccataatttctaatcttgtggctaattttttagtcctgcaagagcagggtagtcacccatcaagaagcggtttgtcttgggaaaggtctgttactgtctttgtttcaaagttaaactataaactaagttcctcccaaagttagctgggcctatggccaggaatgaataaggataggttggaggttagaagcaagatggagttggttaggtcagatctctttcactgtaataattttctcagttataatttttgcaaaggtgatttcaagagtagtggtgctttgaatgcaaagttccagtacaggtcaaggaatagaagagatagggacagccctggagcctgtgcttcctcttttcttaccatctgactggaaagtttttcacttttatattctgtgtccaatcaattggtttgccaaattttgcatttgaaactctaagagaaacccttcttgtatccccaggaagtaaggctttttctcaagttccagcttaataaacataaaatggaaaggtcttattcagctgggacttgagacaaagccttacttcctgggcagaagagattagtttaaattagacaaggaacacaggtaaccacccctgtgttgacatggcagaatgtattctagaacctgctgtgggtatttcataaatgcttgccttggcctgaagctgcaaaaggattactctacacagggggcttaggctcattaagaagagagctgtgccttacctttgagaagagtctcttgttcattcattcaaacctgaaaggatattggtggcagggtgatcagaggaagttagggacagggtgttccaggtagagagtagggcatgtctagggcatgtaggagactgtggttccaatatgagtgtcctactccattcagtttcttggaataataccatagactaggtaacttaaacaacaggcatttatttctcagagtgctggaggctgggatgtctaagataaaggtgctggcagatttgctgtctagggaaggctcactttctggcttgtgaacagccaccttctcgctgtgtgctcacatgctctttcctgggtgcaggctcctggagagagggtgaaaagatatcttctctttctctaatcccatcaggcactaattccatcatgaaggtctcacccttatgacctaatttaaacctaattacctctcccagaatccccacctccaaacaccatcatgtcaggggttaacgcttcaacacagaaatgtcaggggacacagatattcaggacgtgacagagggtgaaataggtattcatggaataaattgaccaaagttagggagaaccagagctggaggaccttgatctccagggagttttagagagagaaggaaggtgaggaagccttccagcagaggtttcatttgttggacaagcatttattgaaagtccctgcgttctgcggtacaccttgctggatgctgtcagccagggtgtacgtggaattcagtttggggaagagaacccatcaacagctgataggttagagcacagggaggtggaggcttttgtagtggtctggagggaggtaataatgaattcaaaatgaaacaaaagggaataggggaaatggatgcagtgtccattccatgtcccaggaacttggtttaaatcctagctcttgcctgttaactgctgaatgatcttggctaagttgtctaactttcagacccttttttctcttagacataaagtggattacagttacaatcaatttgatgccacgattgatgtagggaggagacttaggaatcagaaggtgtctccaggagggatcattgaactgtccttgctgctgttcccttaggaacattctacatggcattgcctcgtaagaaacagtcccttcacttttgaggttaagtgggccgtgttcccttggcaagcctgacagtagcggaacagggtctagaagccaaggtttggatgtgatgctagtttactgattggtctttgcttctctgcaaaaatatctacatctgacaggctcttgttttcttggtttctttgttcttttctgcagttctcagtatctgctgtaagacttactttgctaagtgtttgctcatttgattcttttgttaaacaaaattatgggaggtctttgttctagactggccccaacagaccagtccaaatcaaaatggactcaatcatgctaactgcgatgtaatcaaactgaaactttaaggaagcagagagatcccaaaacagaccaaattttttttaaactcttgaaaacaggagattgcatcataataacaaagtcccctctgctctaaactccactaaaaaacaagtaacctgaaataatcagtcaggttctttttttctatttctgtgtccttgtttccacctacaaaactgttctgctgttttccagtgggatttgagatgaaataagtccgttaacgatggagacagagtgacttcaatgcctaaagttttggtcagcctctcaaaattgccaaatttaccaacctctcaatcttgagaggttgatctaaaggggggaaattgttaaaaaattaaatgtagcctaaagctgcctccttacatattttaagtttggcctaaatgtttctccatacatagtaaactgtgacctaaacacatttgtgaacagacttgcgacctactcttataacaagtagctgagtcttacccaatcgcattagctgagtttcagccaatcgcaggtggccactagttcaaatcctgttcaaataaagcaaacacccagctgtatcccatctagctgttcctgtacctcagttgtgttttctgcatatcactttcctttatctgtccataaatgttatatgacccagagtcgttctgaatgtattctgattccaagggctgcctgattcatgaatcattctttgctcagttgaacgctattaaatctaatttgtctaaagtttttccctgaatacttccaataactttggaaggtagatgatggtggtaagtggtgatattatttcttacattctcctcaacagattggaaaactaagtcagaaagaggttacatgttcaaggcacacactagcttgtggcatattgagaaactcccatctctaggactagttttatctcctataaaacacagctgccttgtgtgtatttttcttttattttactatgatgcttttaaaatgaccatgggttagtatggaaaatagtaatttttgcacgttaaaagtatgcattgtaaaagattgtctgatgtggatgatgagtagtgccacttccaattttatgtggccgtgatgaacttggcataagttctgaccagatctgagctattctaatggaacaaacaaggaaatacgtctcctctgttgttagtttcctataaattctctaaaattattctctgccctgttttgttttggtttttgttttttgtttttttcgaggcagagtcttgctttgttgcccatactggagtgcagtggcacgatctcggctcactgcaacctctgcctcccgggttcaagtgattctcctgcctcagcctcccgcgtagctgggataacagtcatgcgccaccatacttagctaatttttgtatttttagtagagatggagttttgccatgttggctaggctggtcttgaactcctaacctcaggtgatccgcatgccttggcctcccaaagtgctaggattacaggcatgagccaccatgcccagcattggtatgtttttaaatttagattttaggatggttggcctatttgtaaaaatattttaggtgattctgatttgctcgtgggatttggtgtgtgtgggagtggagttgggtgagttggtagaagtgaggaaggtggggagttaagccttgtttccatggaatttgagaggccatcaggatgttatagtgagtataagagctccctccattacaggtaacagaatcctattagctgtaaggacaaaattataacaaatttagttgaaagatctcaattggctatatttgcaattctagaattgagcgacactttattccacaaaattgaataaatgtcgcagtgagtccagcagagcctagcttaatattcagaaaaagggctgaagaaagcagaaacaaagaacacaaagttgaattggtcacttcaaagttactttccttgtaagacagggacatggagacagagcaatagagaaataactggttacatcaagtgacttcaggttaaggattgaaatggagggagcttcattatcatgccaatagcaactggcctgtttgggaaataggctgctatctgtctctcctgatttcttggaaagccagataacaactcagtttccatttggtaacgtggaactttagcatgggtgactccattttgatttttattctgctctgttggggcctagtacaggagcctagtcctaaacagtggcctcctatcatttttatttaagatagccaaggtaaccaaaaacagggagtttctcatcaggactccgggttctctcagggcactcagtgacatatgagccaccagggaccagccctcacctggattctctctgcctcctatcttggcttctctgtacatctactctgcctcctcccctaccctctccctctggcttcctgcacccctgcagtcctcatggtggagaaccagcttgtcagcatcataggtttcaggccagcttcttggagagtggctgggagttctttctttttctatcccaattctcaattcccagggagggcttctggctcacccgaaccaatcataaaccgtggagccattgagtcacgtggccggggtcgtctgtgtgagcctggctgctgtgggccctcactgtcaccatggcctcagaggcagagagaagcggtacccaggaaaggggcggctgagtgaatagccaaatcgacgtgtctatgcccatgtccacgtaggatcaggctccaacatctgcgtgtccttacttaattcagacaaagccagcattgtcgcctttagaacatgtgactcatgctggcaatgggcctagccacaaattaggtcacacacagaggaaagaactgatatctattgatccttcctcctccatgtcagtcactctgctgggcaacttacagctgccatcctaacagtcagaatggcaagcaccactcactgtgtgcccgctttcagggatttaggcgagtcaacagcaccaggagctaactgctgttatctcattttacagaagaggaagtcgaggctcagagatcaaataacttgccccaggtgctagagctggtcagtggcagaggcagggtttgaatccaggcaggcttcagaggcaagtcataattgccacttttctttcgctgatgaggaaagtgagactcagaggctaaggagtgtctagggtcacacaccctgatggctaagaggcccagccaaggttagacctgggaccctgtgccctccaaacgtaggctcttccctccgtgctgcaccctattccctccatggaggaggagatctgttgtatagatttcatttgacgtccccagtgacctgtgagtgggtgggtttgagtggcaggtgacgtgtctgcagattataatagatacttctaatcttccttgtgtgtcttcactttcatagggcagtcttctgtaaccaaactgaaaaactcatcctgtagacttttagcctattatctagcctacaaagtccctctgaaatagggtcctatcttgccctacataaataatcgaggcgggctatgcacagctgggaaatatctggcaaaagacaccagaagcaaagcttcctgctctgctgatcttcagcgatgacatcctttcaggaggccttcaatacatgcaggaaaggggagattaagtgtgaatgtaaatacagagaggccgcccagcagctgctgcatgaagccaccatggtattaaatagcatttgtccactgggcttgagcagaaatgaagtcttgagatttacccctagagaagtggcgggaaagtgtggtctgcagggtggtgccaggtcagagcctagagcaggatgacttttccaggagagatgacacaggtggcctgtgcctgggatcaagattcagaaaggagccccaaactcagtggccctggagtcactgcctagatttgaatcccaactctgacgtccactagctctgcattgcacaagttacttaataatctctattcgttagttttccctctctaaagtggaaatgataataatagtacctgcttttcaggtcatcttaatatttgtttaggtaaattatatttataaagcatttagaatagcgcttggtagatactaagtgtttgtcaaaatatgaatgtataaaatccatatttattaaaagctttgaaattaatctcaggtcttggtgatgacatggacaaagaaagaagtatttatttgaccttcgctttattggtttcctagggcaaccataacacattatcacaaaccaggtagcttaaaatggcagaagtgtattcttctgtggttctgtaggcaggaagtctaaaatctaggtgtgggcagggccacgctccatctgaaggctctggaggacaatcttccttgcctcttctagcttctgatggctccagatgttccttggcctggccaggatcactctcatctctgcctgtcttcacaggaccatctctgagtctcttcctttctaaggacacctggcactggatttagggcctaccccaagtccaggatgatctcatctcaagatacttaattatgtctgcaaagaacttttttccaggtaaagtcatgttcacaggtcttggggttaggacttggatctgtcattttggggaacaccattcagcctgctactcctgctatattcttatttagtttttcaagtaactttgtgctgctcagagatattaagcaatgcacccaaggtcatatttcttgagaaagacagaatcagattgaaacctgactgtctgattccaagtgcttatttcacttctgcagatgtgtagcaagcatctactgggcaccaagcctgacgctgtttggcagatggtgttgactgggttcccttgctctcaaaaagctgacattcagctgaggagacctccacctccacaatcagctgtagcacagggcaggctagtccgtgcttactatgaggtatgaatagtgttcacagctgtgtagaggagggattggtcatggtgggggcaatcagagaaggcttcctggaggaagtagcattcaaactaagctatgagtgtgtggaagagttcaagacacagagaacattccaggtggagggaactgaggcctgaatgatgcaagttgagacaggctcataggacatggcctggggagcagtgaggctggttccctgcatgtctggaaggttgtgaggacatggtctgaggagtagcttggaggcatatggcatgcaggctctggaatgtcatgccgtcagggtgctgcagacagtggagattgagcatcactcagaagaaaacctctaaccctgccaaagcaacatgaaggaggccatggggagcatggattggccactgagtttgtttctggaaattcttgtgtgtatttggacactagtggcttagctcagtcctatgcaaagtgtggcctttggtgccaacagcatcagcatcaccagggagctttttataaatgcagattcttgggacccatcccacacttaactcaatcagaattttcttttcttttcttttttttgagatggtgtttctctcttgtcacccaggctggagtgcaatggtgtagtctcggctcactgcaacctctgcctcccaggttcaagtgattctcttgcctctgcttccagagtagcagggattacaggcgcccgccaccatgcctggataatttttgtatttttagtagagacagggtttcaccatgttggctaggctggtctcgaactcgtgacctcaggtgatcagcccacctcggcctcccgaagtgctaattttctgtttctttttatttaaaaaatatttaaaatattaatgttaaagtataatatgcatacagaaaaattacgtgtaagcttacagcttgatgacttttcacaaatcgcatccgtgtaactagtactcagatcgtgaaaaagaaccatcccacggaggaactgcctcttgttacctggtcactgtctcctgcctcctcccaagggtaacgcccgcctgactttccgtagtatagattaattattcctggtgggatcacacgatatatactctttggtttctggcttcttttgctcaacattttgtgaaatttatccatattgttatgtgtagttgtaaatcttcattctcattgttgtatactattctattatgtgtgaatataccattgtgtgttgatccccctactgcattggcatttgagaggtttttggttggggccattatgcttagtcctgctatgaagatttttcctatgtgttttagtgactatatgtacacatttctgccgggcatatatctaggaatgaattgctgcacgatggggtatgcatttttttcatctctagtaatactgccaaaaagttttcccaagtgcgtatgccaagcaaattttttaaaaacattattatggagaatttcaaatatgtataaaagtaggaggagagtctaatgagtccctgtttatccatcactcagctccaacaactttcaactcattaccagtcttactctggttctacctctattcactcctcctacccccttcttttactttgaagcaaatcccagttgttctgtcatttcatctgtaagtatttcagaatgtgtctccaaaaataagaacaatgctattatatgctgtggaagttaacagtatatcctcattataatcagacattcaggaaatgtgcagttttattgattttctcatagctgcatttttaaaagttttttttgtttgaatcaggatacacataacgtccatacattgtaattggttgatatatctccttacatctatttattattattattttgtttgagatggagtctcactctgtcacccaggctggggtacactggcgagatctggactcagtgcaacctctgcctcccaggttcaagtgattctcctgcttcggcctcccgagtagctgggactacaggcatgtgccaccatgcttggctaatttttgtatttttggtagaagcggggttttaccatgttggccaggctggtctcgaactcctgacctcaagtgatcccctgcctcggcctcccagagtgggggattacagtcgtgagccactgcgcccagcctccttacatctatttttgatttataaggtgcacctttatgtctttttccctcttgcaatttatttgttgaagaaccacgttgcttttcctacactatttcccttaggctgcattttgctggttgcacctttatggtgttctttatcatgttcctctgtctactgcatttcctgtaaactagtacttatagagatcagattcaggttcattttgggggaaaaacacctttatagatagtgtcctacatttacatcaggagatggatagtttctggttgtatcttttgtgacgttggcagtgatgatgatcactgtctagatttattcattcataatgggcccaattctttcaaaatcattcacaaacatgctctcaaatatattgcaacttttaggggaaaaggggggatactttgcatattttgtaacttaaactttttacttaacattatatcacgggcatctttccatgtctgttcctaatagggtgttgcctaaattaagaaagttttttaagtaaagaacttaaaacattcaccaatcttttgatgtagagccaaggcttaaaatttgtttttgttaggcgttaattgttagaagtttcaaacttagtgtttctaatataaatcatactcaaaatgcatcctttataatttgtggtttctttgttttggtttctttgaagtatagcaaaaactaaaacacttaagagttcagagttctagatttttagtttgttgtttgtctccccagacttttaaaatttactcacatgtaatttcattcaaatgactggcctttaataagtctttataggacatttgacttaggtttttaatgctccttctctttatgttcatgggtctttagttggtgtagtatttatttattattcattaacaattgtgcttaatatacagaaaggggcctgggagcaaacccaatgttatgggttgaattgtgtcacccgaaaattatgtggaatttctaacccgaagtacctcaggatgtgactttatttagaaacaaggttgtcgcagatgaaattagttacttttaggtcatacaggggtaggctgggtccttaatccaatatgactggtgtcttcttaaaagggaaatttgtggccgggtgcggtggctcacgcctgtaatcccagcactttggggtgccgaggcgggtggtcacgaagtcaggagatcgagaccagcctggccaacacggtgaaaccccgtctctactaaaaatacaaaaaaattagccgggcgtggtggcaggcgcccgtagtcccagctactccgggggctgaggcaggagaatggtgtgaacccgggaggcggagcttgcagtgagtcaagatcgcgccactgcactccagcctgggcgacagagcgagactccgtctcaaaaaaaaaaaaaaaaaaaaaggaaatttgtaaacacacacacacagggagaatgccaagggatgatcacactgctacaactcaaggaaccaccagaagctaggagagatctctaacagatccttctttagaggcttcagaaggaccatggccctgttgacacctggatcttggattgctagccttcagaactgtgagacaatacattgccagtccgttgtggtactttgttacaacagcaccagaaaactaattcaccccataagtggataaatgagctgtattcgcagagggccgagagcatccgacagagtggtcacctcatggggagggacagagcacagccatgtgttctgagggacggaactgatgtccatgtcatctggtcagccgagaaagtgggctttagctcctgggatctctatcctacatattctattgtgtatacatacatttgaatcacattatctatttaattatgaagattcaactccatgtgctgaaaaggaaaaagtgaaatagcttaaatagtacacatgattcttcctactgttctacagctagatcacatagtattctagaagaagcccttactctcagggttgacattccgttgttgttgtttctctgtagtcagctgataatgcatctctcctttcttaaaatcctaatgattcatttaaatctctggatgagttccagtggggggctccatttttcctgtagaattcaaagtgcctggattttgtgatggctatttctatttcagggacttaacaatttatttccaagataactgcttgttacttccatgggaaaattcaagtttataatacttaagccaacaggagtccccagcttaacagtaactacttaacagtaattaaatatctgcaaagagctcaggggcaagggacaactgtgacatgaggaacactttgtgtgatgcagaccaaaggtgacaagggctcctgggtatacccgaaatgagaagacatagtgttaaattaaatccgaatcatcgaatgggctcaattatatatgttgctttcgtgttcaatatttgctaaaataatttcaaactaagtttattctaaattttaaaacaatgtatatatatgttgggaaaaacgcaaatatcacttaaaggatgaaaaaggaaaagtaaaataaccctacttcactctccagaggtaattaatgatcaccaagttttgtatgccaccattcattcattcgtttggtagatgtttattgagtgtcaggctctgttttaggctctgggaaataaacatccacaaaatgttttagacacacatgcacacacacacacatatttttttttcaaaaggaattatttgagagcccaggagtttgagaccagctgggcaacatggcgaaaccccatctctacaaaaaaatgcaaaaattagccgggcgtggtagcacgtgcctgtagtcccagctacttgggaggctgaagtgggaggattgcttgagcctgggaggtaggtggaggttgcggtgagccgagattgcaccactgcactctggcctgggcaacagagtaagaccctgtcccaaaggaaaaaaaaaaaaggaattatttgacatgctcttctgctccttgcttttaaaacttaatatatctggtagataattctagtcatgcgtttctctttaatttggaaaaaaaatgcagggataacctatttatatgactgaaaattcagaagtacaaaggaatatgcagtgtgaagcttcagtcccattcctgattctcagccccccgcctccctttataggcaaccattgttatcagcgtttgtgtgtgtctgtgtctcttcagacctattttatgcatgtagaagccaacatgtgtatatggtcttgtctccatcagtgaaaaaatactgtatatattacttggcaccttgctttctttttaaacatacagcttgattattattttatattagcacctagagagccctctcatccttatttttttagatctacccctaacagtcttactaccattttttttaattgctgtgaggtagtttattatttatcaattaatttcaaccattttgctgttatatacattgttataaattattttagttatttcacaaatatgcaagtatatatatggggtaaactcctagaaatagaattgccaggtcagtttataatttcatgaatttgcactaagtgacttttcactgaggctggactgcagtgacacaatcgtagctcactgcagcctccacctcctgggcttaggcgatcctcccaccttagcctctccagtagctggaacacaggcatgcaccaccccacccgactaattttttttattttttagagagacaggatctccccatgttgcccaggctggtctcgaactcctgggctcaagccatcctcctgccttggcctcccaaagtgctgggattatagatgtgagcttctgagcctggccagtttgcacttttgatagatatcacctaatggcactccataaatgttgaaccagtttacctatacatcagaaaaatgcagaaacagctcactgaattttgtctactgtcttgtggggtggacatttctattttatgtacatggaaatggaggctcggagagggtaactcacttagccaagggtcacatagcttgtgagtgggcagagcaggaatttgaactcatatctgactgatttcagaatcttgtttttttcattatcctcttagactaggctattacataatacatttaggagggttgaacaatgcattccatgaaaatgctttgtaagttccagtacttcctagatgtctagaaaggaaaaacaagtggagtgattctgaacatttgatataaaggttctgtccaggtattactcagtggagtttagcccattcccaaggtacctttttggacactgtactgcttgttttccaggtgtgtcttccctgctgcaacatgactaggctccagagttaagagctgggctttatgcctcctaatgtgccttttaactgaaaaattatctttgttttgtttagtttatttttagtaattcttctatttttggaagcttatttacttttttttagccttttgaggtaaattattttatttttatgcttatttcctaataaatgcatttaagtctgtaaaattactcctgaggacagttttggcttcctcctctaagtttttaaatgcggtgttctcatttttttttttttttcccggagatggagtcttattctgtcgcccaggctagagtgcagtggcgtgatcttggctcgctgcaacctctgcctcctgggttcaggcaattctcctgcctcagcctcctcagtagctgggattacaggcatgcatcaccacgcctggctaatttttgtatttctgtagagatggggttttaccatgttgtccaggctggtcttgaactcctgacctcaggtgatccacctgcgttggcatcatttttcatttctgatagtttatcatttgagttttgattcccctctttactcatttggctaataatattgaatgaattttatctttttgaaaagcagtgattttttaaaaagtatgagttgttatagttagttcagtaaaaaatgttaaagccttcagtggttgtgttctgaagtccactgggctttgtcctgagctgtgagccggatgcctagagcgattcataaggccccttcactctctcctcactgttttctggaactgcacagtgaaacctgttccttccattacagttgttgaggcatttgttgattatgtcccagtttcctctttgtgagagcccttggaaaaaaaccttctttctagtctttttttcctgaacaattggcctggacagtttcaggtggctgctactgggaagttttgaaacagtgaatcacccttcgccacaggtataggactctgctgtaccttttgcttcgtatgttccttgttcagcaaacaggtaaggaatgaatgctgtgggggatggaaccatcagtcagagagcatctgactctatggagctcctgtttcctcttaggtccccgggcttgcttttctttctttttctttcttcttatttatgttatcgtaattctatatgtatctctgtaaacctaggtagatataaatagaaataaaaatcctaatctttcttgagtgggattaaaataagaagagtcgataatttaaaactagagaagggaaatagagaagatctgttttccccatcagcctggatgaaagaagtttctttagccttgcaaggagagtggtgattagtttacagaaaatggctggagagacaaagattctgttgaagaagcctgtggagaagaagcttcttagctcaccctgggagccagaaggtggtgatgtgactgttcacatttggtttttccttagcgttgattacttcatgtaggagtctctaagttcccgaattatgatttgaaatgtttcaatctgatgctgtcactgatgaaattaggacatgtttactaattgagatgaaggctctcttcgaacctgactgcatcctgggaaagaggaggttgtatacgtttttatatcctcttggaaactggctgacccagaagctcagcctaatgagatttggacagcaggcttccagaagggtgattgattgtgacatgctgagttttatgtgccagatctgtgtacaaatgactgactacccggatgcctgcctggcaaggaattgtgtttctttaaagagccattccaaatataacttgtgcatagaaataaaactgagaagaaatgagtatctccttctgaaattcaagtaaatagctccctgacttcagaaatgacagcttcctaagcgttgtattgcaaaaacaggagcaggccagtcttcctgaagactaatttgctcgcatggaaatcgcatgctctcgtgtgagagtgtatttcaaatctgtccccttctgaccaccaggactgccctctcccagctgaggctgccagtcccctggaccatggccagggcctcctaatgggcagagagaatgggcagcagcttgcagtagtagccccaaacccctgtaatagctaggccagccgactccctggaacatgccaaacgctcctaagcctcagaccctttgcttctctgcctattccctttgcctggacgactcttctgcatggctcgtttcttttttttcttctttcgattttttttttttcatggcttgtttctttccagcctttgctcaaatgtcatctcagagggacagatcgtcttccaccccaacacccctcaccttgcttcatttttctttatagcatttattctggtattacaataaatatttgtttatctcacttgatagactctaaacatcttgaagaggggaacactattgattttgttcactcctccatacaatagccccagaatctggagtggtgcccacaacgcagcaagtaccccataattgttgcgtgcaagagcggatacactcttgtgtaccctctcagggccccctcctcttccctttgtgagcacccacagtcctgcgttataaagatcctcttgccctcctctgccaccatggttgttgtttggtggaatcctaaatctggttgcagcgttttctggttactccacttcctcaggcctacacccaagcggccgactgctcctagaaacaattgcgactgtgccagctggactctcttaaatgaatatccacagcccccacgaggatgctcgtactcctgacagctctgacctgtttctcagtcaatcctctttcctaaccccctgcttgcacacgtcctcttctcttctcaaatctctaccctgaactctcctctgccagctagagaccttactcttcatggagaatatggacagagtcacttgcagactcactcacccctccctgttttgacttccagatttctactagggttagatatgcaggaaatgtttttgtttgtgtttaggattcttttcccagaacattcttagccaaaagatacgattaatttccaagtttttgaaagaattatcaatccaccagaaggtctgtgatgtttatgccaccactagtaatgtgttgggtatttagaaaatggaattgtaaaggctatgacattgcatttttaaaaatatatatttctcattaccgtcaaggttgaccgttttcacatctgtttcctttctaaggggtttgtctgtgaaatgcctctgcctagctatgtagcagagtcttagtctttttattattgattccgtgacagcctctcttcagacgaaaaccctttccctaggctgtttaccgtgaattggtattaggctatttggttctagggaatttgtgagtttggggagatcttccgatcagaaataattattcaattccctacttccattttgtagatgagaacacaaaggcccagataggagaagcctgctcatttaagaccctgcagccaaatgagagctggtaggataacgaagccagaaagaggacaaggggaatttttgcctgggaggttatctcagcacacaagtgatcctcctgcctcagcctcctgagtagctggaactgtagacgtaagccaccatgaccagcctaatttatcaagaatggtactgtgattatgaatccacattttgtttctccaaggaaaatagtcttgcctaggagaatgctaggttcttagatctcatttagatgtctcatttggggcatgcaagttttactaagcaattgctacaggtggacagcactgggttaggagttacaggatttgtgtggcagagagaacatcttctgctttaaggaatgaacagtctctctagcagagtagacaaatgtatttacgtagaataaatagtccagggaacatgtaagccagtggatcattaaatgtcacaagtgagttatgagttgcagggatttagtgatgagggaaatcagctgaggacctcacatttaactttgaggatagggatgatttttttcccccccggaactcgagtactgaccattatagggagggttttgataggtggggaaatagcatcagcagacctcagagggggcatgaacaaaggcatgctttgggaggtcggggatctggctggattgaaggggtctcatttgaagggagacataattatagcaactctattaattgctcaatatgtattgggcatcttgctaagattttcacacattattttttaatgatgagtattattaccttcattttacagatgagaaaattgaggcccagggaatttaaataatttgctaaagttacacagcattaagtttctgacctggaatttgaactcaggatcacttgatttatggtgaccatatatttgtcatttaaaccggcaccttttgagaataaaagagagcgctggtaatgattatgctagaacagcagatgtaaaccagggctgtctggacaaatcaagatatgtgtttggtcactctgactccaaagcctgatttcactttgatttgaattggaagggtagggttacattgtgggtagctttaaagacaattgatggaggccgcgtaaaaggggttagatagactgattaacttgatagcgctgtatagaatggaatggagtagaagagagatgagaggccaaaagaataattagtgagtgacttcattaaaagtagggtagatgtgatggagatctgaactggatgatggaatgcaagggaggtaagacgtcttataaattagtttgttttgttttgttcgagacagagtcttgcactgtcacccaggctggagtgcagtggcacaatcctggctcactgcaacctccacctcccaggttcaagcgattctcctgccttggcctcccaagtaactgggatgacaagtgctcaccaccacacctggctaactttgtatttttagtagagtaggggtttcatcatgttggccacactggtcttgatctcctgacctcaagtgatctaccagcattggcctcccagagtgctgggattataggcgtgagccactgtgcccggccttataaattgtctataatgaaaaattccacacctactcaaaaccagaaagaatacagtgaatccccatatgccagtggcccagataaaaaattatttggttttatcatatttgctttatttatcttttctttccatttttctttgctgaagcatttaaagcaaacccaaacataacgtcattttccctcccaccacagcgtgcatttctagagaatattgactatccatagtgtcattaccaaattaccaaagttaataagaattctaattattttactcttaagtatcatcattaagtttagtgcccatattttctcagttatcttttttactgtttctttccttcccttgaatcggaatcacaacaagggagaaagaaactcttaaacattgtggtttttctccaaaactgagtcaaaaagttttgagcttgataaacgaggaaaattagttagctggttaactggcaagtggagtttggaaaagggaaagtgattgtagagaaaaacagaaagaggtccaacaacttttttcatttcagacttttattttgaataacctcaaacctgaaaaacattgcaaaagtagtacagatgaacacctgcttattctctacctggattcaacagttgttaatattttgccacacagtgaaatacttcagcatgcacctcaggtcattttcttacataaccacatgcaaatatgacatgcaggacatttcgcattgttatgacacttttgtctcccaaacagttgatatttacatttctcaaatatttacattaatttttttatagcttttattcaaattaaatatttcgtctgtggcactacataaatgacatgccctttcactgttatcacagtaggaggctcatgatgtcaatttgtctcattactggtgaaattaacaatgtgggtaaggtagtgtcttccagatttttccatatactatcctgttccccaacaatcatttgcctagggcttttagcattcataggttattcttgccttaataaatcacttaccgccaggccctgtggctcacacctgtaatcccagcactttgggaagctgaagtaggcggatcacttgagcccaagagtttgacaccaacctaggcaacacggcaaagcctcgtttctagaaaaaatacaaacattagccaggcatgttggtgttgtgcacctgtagtcccagctactcaggagactgaggtgggaggatcacctgagcccagggaggttaaggctgcagtgagccgagatcacaccactgcactccagcctgagtgaccaagtaagactctgtctcaagaaataaaaaataaagcatttactatagtgctgtaaagtggtggtgttctgtcacttcttttccatttattagggggcattctgctgtgtcgggagcattctgataccatgttgtagaaatggggaaatctgcttatttcagtatctcccaaggacctgatggtgattttgtgagtggccagaacagagctcagggctgtggattctgtgggagtgcagtgagtaaggctggagacgtggagatgtagaagcagtcggaaaggctgggtacttcttcagttgccaaggaggcttgatttagcctggcattctcagtggaggttcacgagcgttcaggctcagctctcacaaagcattccttttcatctccacactggctgtgggtttccaatcccatagctcataaacaggcgtagatatagatccaacagagaagctggtttgttttgtgtttagattgaaagaaggtagcctttgggcatttcattccctcccttttcatttggggttttgttttcggacattatatagaactttatgtaggagagattcaaagcaaattagaagatgtcatgtttgtattggattctaggaagtttcttggagggctaccgagttcttacaaatcccagtctcatttggagtttctgactctgctctcagctgcgatgaataattggaagactctgttaaaatgccgggctcattaaaccagggtcagaaacctggttccagtttgctcggctgggagaaaaaccattccgtgtccaaagacagtctgggtcttgaccacctgaccaggcagccttagtggctctgctgttgggcacaaaggggaagacactttacagtctgaactcacaaagcacatcctgatgaggccttacgtgttgcactcaacctgtggccaaaaccccacactcagcatcttgtagcgtaaaatcaactcattccgtgctcagagagggcttggcacacaataggtgccttcttcacctcacaggacgatggatatttagagtggttagagagttcacagctgatcatttgttaatgtttcccagcactcaaaaggttgttagtgataataatggcataacatttatagaagaggaaacgaaggcacagagtggctaagccacctgcccaaggccacacagccattaaatagcagggctggagttctctgctcctaactgctatggtaattgcctgtctttcctcatctcccatcctggcatttttatttaagcagtgccagttacttgcatttccttcagcctgtcatgttgtctcatgtctgaataactttgaacatgctgttccctgtccctggagttttttcccttcacttagtagttggatatttctactcttcttttaagacgcagctcacatattatctgctctagtaagtctttactcctcataactctcccaacctagccagctgatgctgagccttatatgcctgcatctccccacacagcacacctactttattttgtttactatctgtcttcccagtaggcagtgagcctattaacagcatggattgcatcttgtcatccttgtatccttagaatgcagtacagactctgaaatgtatttgtgcctattaagcatttttgggaatatagtctgaccatgccattaaatatatgaagggatttcaaaacgttcatggaaaatgtgtatatggaaaaaactatgcatggatttcaaaatttttttgcatcagaataaacttatattaacttcttctaagatatctgaataggtactagtttggttcactaagaaggataagacatcagttttaaaatagcctggattaggctgggcatggtggctcacacctataatctcagcacttttcgggaggccgaggtggggaaatcacttgagcccaggagttcgagaccaccccgggcaacatgacgaaatctcatctcttcagaaaatacaaaaattagctggatgtggtggcacatgacggtagtcccagctactcaggaggctgaggcgggagagtcacctgagtccaggagatcatgtatgcagtgagccatgatcgtgccactgcactctagcacgggcaacagactgagaccctgtctcaaagaaaacaaaaaccaaacaaacaccctgtatcagtgcaacatgaattctaaaactgaagcaagaacaaacatcagtttttgtgtgtgtgtgtgtgtggtttttttttttttgagacagtcttgctctgtcacccaggctggagttcagtggcatgatctcggctcactgcaacctctgactcccgggttcaagggattctcctgcctcagcctcccgagtagctgggattataggtgcccaccaccgcgcccggctaatttttgtatttttagtagagacggggtttaaccatgttggccaggctggtctcgaactcctgacctcgtgatctgcccaccttggcctctcaaagtgctgggattacaagtgtgagccaccgcgccctgcccaaacatcaaatttatggtgaagcttgggtggaagaatagtgaaataatggatgctttacacatattgtgtggggacaatgcctcaaagaaatcacagtttgcaaatgggtaactcattttaagaaggaatgagacaatgctgaagctgaagcctgcagtggcagaccatccacatcaatttacaagaaaaaattcatcatcttgttcatgtcctattgaatataataataaactatatatacatatcttatatatatatataaatttaaggagatcagagtcctcttattcagtacaattatgactggtagttggtaagtttcttatgacaaacactacatgctgaaatactgtaaaaattgttctaggtcacatatcctacataagaatatggtgagatctgatttacaggaaccagcatttttaactttgttttgtcaagaagtgcgtatttcttatttgtgggtggggggagagggagatagagtaccagattaataaaattattttattttaaacatttaattaaaacaaatttacgtgagctcaaactatatataaggaaaatcatttccccttcacaactgcttctaatcatactgcctgtcctggggaagccattgttgacagctgagcctttttccatgtagaacagaacagggtttggctgattcacattctcacagcagcgtttgtgatctcacatggacctccacacagattcaaataacattcctttcaaaggtcctcccagtaatctttcccccaggaacagcagggacaaaggctgtaaatggaattatgctttccaggaatgtcttagggaaagaaatgccgtcggaatccacaaatattttggcagtggtccttaacccccactctctggattctccaggggcggcagcacaggaacccggcccatggcccatcgcactgtcggggagccccagcccagccgccccatcatcagggtggctgcactgcaatggtcaggatatcaggctccaggagatccctggagcgtcccttcaccctgtgcaacctgagtactcgcttcctctaaaggaactggaaatgataatcttcccagttacgggctgggctgtaccacatgctacatccaggtaaagtaaaatcacagtccacacgtggctgtgagcacatggctgccaggaagctacacaggccagactggtccagatgttttcacaggaatagcttcccgggcagcatgttttctgagctccttacttgcagagtgagttcgtgccaccattaattctagtaaaagccaacgtgtgtcagtcctgcagggagaagccactccagtcagctaaaggagaggaactgatataggaaaaaagtaccctaaaggtgttcaacttgcattttaaataaaaaaaaagggagacctccctctccagggtactttgttatatttgtggaggcaccggggatacattcgacacgtctgataaatatggcttggcttctaaaagtctgaagcttcaatccttttatcaaaaaccaaactgaacagtttagtcatgtggatgtgaggatttgaagggtttaacgaaagtgctctgagaaactgaaaagtacagccctgttgtagcagaaaatggtttttcaatgcatatcactcaggtactaaactcagacaggtgcaactcagccacagcataaggaaataggctcagtcaggaaatttggtgaactccaggacccgaggccatcgtactgtttgggtccccccagaatcacttatttggtctcagtttcacccataccagccatgggaaaatgtgagcccaggagtggacagattgtgttcacttcattataaataatgaacacatttaaccaaatgggagtgctggaaggtggaggtgataagtatatttttcagagaagccacacttaacccttcaagtgacaaacttctttggctctagtattttccatggaagtcttttcaaaaggcatttatttgaactcaggcctttgaattgagtgcacaccaaatataaatcaactttttaagggggatggttaaggatccgcattcaagatccttgtcctcagagactatgtgtggcctcttgcccttgtgctcactgggccccgattgctgccctttgcttggtagggggctcaggttgtcagcagttgtcattaatgatagttcctctacctgctctgaaacacagctgctcagaggtgttctgctccagcacccatgaggtataaccatccagcaggagcaatagctggctggggtggagttaggccaggctatagtagatgatgacgtaaacaaaccttaatctagtgttgctgctaaaagtattttatagatgttgttaacatctacaactagtttaacgaaactaattgtaacaaaactaattgtgatgaaactaattgcaacctttaaatgaaaggcagtaccggccagacactgtaagtacctgcccggcatggtggctcatacctgtaattccagcactttgggaggctgaggcaggcagatcacttgaagtcaggagttctagactagcctggctaacatggtgaaatcccatccctactgaaaatacaaaaatcagccaggtgtggtggcatgcacctgtaatcccagttacttgggaggctgaggcaggagaatcccttgaatccaggaggcagaggttacagtgagctgagattgccccactgtactccagcctgggcgacagagactctctctctctcaaaaaaaaagaaaggcagtacccttgataaagtgggtgggcctcatccaatcagttgaaaggcctcaagaggaaaaactgaggtttcctgaggaggaagaaatcctgcctcaagactgtagcgtccactcctacctgagtttatagcctgccagcctgttctacagatctcagacttgcgaggtcccacaattccttagaataagtctctgaatatctatattactgtctcctattggctctgtctctctagggaaccatgacagacacaggcagttattcccagtgtttgggaatgagggtgggaaggagtgcgtggttggatgagggcctagccctctgcctattctagtcaccgaagaatcaaatgagcaaagtcacctgtgtcttgagacagagtctgtgatgactgagtgctgtctctgaagtacccactctgtgtagacagtgtgattccgtgagtcgatggggtcattcttgtctccctcgagcttgtgatttggagctgaagtgactggctttgcaccagatgagaggcaaatgcatgtgtatttgtatgtcttaaataagttaaaataaaatatctaagggttgttttccttctttttcttttccgatcaacctaacagaagtgattatatctcctacgctgattcaatgtagatttaaacattttcaggcaatgaattggctccttaaacattgttttccttgatgaaaggtaaattgagttaagctatgcaaataactagacatttctgaatttgtttaaagtcatgaggtattttcctatctaaagaagacaaattttcattttggaaagtggaatcaggaagttctaaagcataaaaacaaaacccagcgggcctagcccaatgagggaagacaagactcagagggagagttacctctgtttgtaatttaagatcccttccatatgggaaccctggagaaagtgctggtggccacatataaaccatgtaaatgtttatattttcaccaactggctctgcctcttgagttaaatctgcattctcctccagaaagggcaggctggacccagaagctggtgctcccagctcattttaccagccatttgtactcataactcttggagattctactgagatcttaagccaacttagccttgacccctatctgatgcactattcccaggataacactgttacctgagaacattttccttaagtattttggcagtagagacttgtttgaattggtggtttaaactggttatgaacatgtgttttgtagatcaggacaattcagttggtaggcttattggatgtgtctcaggcctgggatgactctccaagctttgtagaccatgaatgagaagcaggcgggaagagtgattcatctgagcaatgctccactgtatttggtcatgatttttatgagtgcaagtaccttttcatgcattctcttttatgaatacaggaattcatatatttcagtgaagatgtcttaaaaatctcagtgtatgtgggtgctggtctataagcttcagtgagtctctgcatgtgtcatgtgtatgtttactttctgctgaaaaaaaagttttgttttgttttttctcattctttgtttagtcaaagctgacaggataggctactggtagaggaggcacgtagctagaaaatgttgggggtcttaattcggtaaggtgtgttgtattagggtaagcaacaccctttgctaagagaaacaagcccaaatctcagtacaaaatgtttgtggttcactgctcagtccaacatagaggctgccgtgagttgggtggtgtggtctggggactgcggctcgctctacctaatggttctgccagccacttgggcttcggaattctccactggatcctctgtatttggctgagagaagaataaagagggcaaacatgcagggtcttttgggaggtttcagaggccaggcctggaagtggtgtgcgtgtatgtccatctgtgttccgctggccagaactcaagtcccctggtccctcttagctgcaaaagagctggggaatagagtcgggctgtgggtcaaggaggaagaggcaacacacagatggcacgggacactttcattctgtgccacttgctgagggccggcagggaagagtggagtgttgaaggccgtgggatcccagagtagaggtgggagaggctaacatgtctcttagcatctgtagtgctccgtatattatataccaggcactgtgctggacacttttcttggggcaagatgcctctcaatcttccatagcctgtgtaatgccagcacagccagagacttaccactttttgaaccatgtagatttgtgttttttctgggcttgtcagttctacttggtttgcctacttccccctgtcccctcaggaattttgtctagataggggcaagttgtgaaatttatttggaagtgcctgcagaaagtcttatatgcctggaaagaaacaattatagttgcattttaattagtatttaatttacaaaaatgatgcatgtacaaacctagtttctgcaagtctctaaattgaatattattttattctgtgtctttttggaaaatatgaataatctctttcacttgaacagtgttgttttaatgtatgggttaacatattaaatccttagcacagagccacgtgcaagaccccctgaactatgtgtgtttaagatggggccaccaaaggcaaaagagggaccctgtggtctctccgcagagaggcacttaacactatttatgctcttaattaatcacatttctaagtaaacacaagtgcccaagcaagctgacttacagacactatcagcaaaatctttccaagagcacttaagcttggcacaaatctgataaatctactgcaaacaacaggggattttaagttctctattcattttacaagagaacattatagggatagaatttcactaggcccagcttaggccagaatctacatatgtattcagagaagccagtttgggaacagaaatatcttagtcaaagatgagttttgactcacataaagaagacggatctgttataatatatgggtcctgcacaaggcacatttcttattttagcttacttccgtggctcagacatcacaaaactcataaatcgcacacatacgcacgcagcctgttggagaagctcctgctgcatgtcctgttgaagtgtagtgagaaactgcaagagaatcccacgaagctaagacatcctgccagcgctgtcccctgacctggggggtgtgtgagagggagacagacagagaggccctgagttccagaactcatttctgcagacttggctgctggccacagaaggtccatactgaagttcacagggctgctcactcccaggccacacaccctggagaggctggcagtcagagctatggacagagcaatggatggagaggcgcagaaccacatcccagacagcgtcatggggcaacctgtgtgctctcaagagcaggctccccttgttttctgtatttcgggctacagtacacatccctgccaccttctcagcatgtgcagtccagaaaaatatccattcaaagtggggctcccgtgatgctgccagggctggttctcatgtggggcttcaattctcagtctcttgccttgcgcttggaggactgtggcagtgactccagaccaacgtcctttccagcaggggcctggtgaagggtggtgttagtgagcatttctccccggaaaaagccaaggcttagctgggtgtgatgtgcatgcctgtggtcccagctactctggaggctgaggccaggagtttgaggctgcagtgagctatgatggcaccactgcactccagcctgggtgacagaatgagatcccatctctatgaaacaaacaaaacaaacaaaaagcaggaaaagccaagacttgagtctgttgagtctgtctctcactggctgaagctggattatctgttgactcctaaaccaagttataggggttgggtggatatgttacttggctcagaccagaaggtctcactcacacactgtgggtctcagatccgctttaactcttaaaaattattcagggccccagagagattttgtttatcaatatttatcatatttgaagataaaatggagaacaccttaacatatatttgtcaattcatttaaaaagaacgataataaaccctttccataaataacttaattttgttaaaaataactatagtttccaaaagcaaaaatcatgagaagagtggcattggtttttgcatttttgcaaatctcttgaacatctggcttagtgcaagtcagctggattttcatatctgtatctgggttgctcccgttgaggtactgggtgttagctccgttgatgtatagggtgaacgtctgctctcactgagataggtagttgtaaaagagagcagtactttagtagtcttttcagataattgtggatatttttctttaataccacactaaaactgtgcaagtgctagtttttaaaggttatttgcaatgtggaatctggaaccatgtgaattaattttttatagtttgatctattaacagccactggtctgtcttccactgtatttatttattaaaaaaatttaaatttgtataaatttatggggtacaagtgtaattttgtgatgtggatacagcacatagtcgtgaagtcagggcttctagagtatccatcacctgaataatgtacaccgtacccattaagtaatttttaatcatttaccctcctcccacctccttgtcttccactttgaatggatctttttccacgcaggcttttgtgacatgatgcatgaatcttttggaaaatattcgttcactgagttttgcacatctcccaaatgtggacatatttcattttatagtatcaaaaattctttaatatccttactgatcacgttagaaaggcataagtactggaatgctgtcaagctcatggtggtggatacgcactttgcaaaattctgatttttgcttgaaagctctagttttatcattggcaacaaatcctgtcagtggcttcttaaacttgcaagctccacttctttcatttcagagaaaatgctggccaggcacggtggctcacgcctgtaaccccagtgctttgggaggccaaggtgagaggatcgtttgaagccgggggtttaagactagcctgggcaacattgtgagacaccatctctacaaaaaagcaaattaaaaagttatctgtgcatggtggcgtgcacttgtagtcctagctatgcaggaggctgaggggagaggggaggataacttgagtccaggaatttgagccttcagtgaaccacgattaagggctgagattgggtatatctaagtctgaatagtcacaatttgtctctcagcctttcatatatatatatatatatatatttatttatttatttatttatttatttatttatttatttgaggcagggtctcgatctgtcacccgagctggagtgcagtggcataactgtggcttagttgaactcctgggctcaaatgatcctcctacttcagcctcccactacaggtgcatgcccccacacttgactaatgtggggtctcactatgttacccagttgcccaggctggtctcaaactcctgggctcagcaatcctctgtccttggcctcccacagtgctaggatgacaggtgtgagccactgtgcctggccatcaacttttcttccaagtaaagatggtgttccatgaaaactgtggctagttgagctcacaacttaaaacacttgcacaaatgctttttgcacattatactttgatgtatatgaaagagctttatgtgtagttaccatttttttttacacaggataataaaaagagctgtactcataggttgaaatttaataaaaataatttttaatgctttatcaagagccttcttaagtgtaactgactattgaacatgagtgcctgatggcaaagaatataatgagtatcagctgccatcttgatttgtgccaaatcacttgcattcttatctaccaccgcctttgcaccctagtgcaaatgtcaacacaacaaaaaaagcaattaacatcttgtgttttgacctcatggacaccctgaacatgtcttggggattcccacagcccatgcttggagacccgctggttcagacccgtgaggatctacctttgaggtgctgggctcacggcttccccagtccacgtggcctgagaggatggaagaagcagttccccagaagaaatccaaagtttttcattttgtttttgtttgtttgcttttgttttgtaagggaaaaaaaaaagtgaatggatattgaatggccaaaataacagtcttccacactgcacttaccatggatactgttctgtgatctgcatgctctaaattccttcatataatgatagcaggtatataccatgtgcccactgtgtgccaggcccggtgctgttttatttataaatgttttctcacttcaccctaagtggtcctttgagagtagggacgtcttattttgcacatgagcatgttcgtagcagttattcacctaagattagacacacagttaagtggcggagcttggattcccttctaactcgttgctagatcaagatcccaaattgcaacatattatgctgtgcagctaagtatattttaataaacagatgatttctcagtttttaaatcaacagtagactctttcattcagtgaaattttaaacaatgaaatgttaaagcctgatctgtaaaacagaccaaagtaatgctgactggttgaagggggaagtagcctgtgttgtcggaaggggattagacagcctcagtgtccctctggtccttgggcagcccccgaggtactgtgaagaaactattggggttccctgggtacatatcagtttgaaaatctctgttcctgcagcataatctttaaacatcagcccatatctctgagttgtagaggagaaaagttgttttgcagaaagccttgtgggaaagtggcttgctttgttttcctatatgttaatctgaacttttttgcatcaaaaaaggagttgatgtagtgtttcgtttgcccccccattagtacatttcaggcgtgatggatatagtgatagcactgcagaagctccttcctggggtgagagagagtgtacaggtggcgggaaggcacccgcacaggagaaggaagtcctgagccttggtctggcctcctcacgaatcactatgtcaccctctctgtccatgtctcagttgtctcatatcattcttagatggctgctcagggttaagaggtggctcttagtcttttttggtcccaggcctatgtgagaatttgcaaatactctaaatgagtatttatggaaaagttccagtttgcattgcataggtttttatgcactgcttggagacaggaagagaaggatttcccatgttaagaactcgagttgggttaattttttttttttttttgacagtctggagtgcagtggcgcgatttcggctcactgcaacctccacctcccgggttcaagtgatcctcccatctcagcctcccaagtagctgggattacaggcatgcgccaccacgcctggctaatttttgtatttttagtagagacggagtttcaccatgttgagcaggctggtcttgaactcctggcctcgggtgatccacctgcctctgtgtcccaaagtgctgggattacgggtgtgagccacggcatcctgccgggttgagttaattttaagattcctttcatccattcctttgtaaaatattaatatcaggcaaatgtggaattattgaacaataaatttcatttgtgtttccagacaattaagaattagcaatttcagcaaaatttttataaatgattattaataagctgtatgcggtttgctggtaaatattaaggtgtattatgataaattactaacttaaaaatgacaactctctaattctgtcactgacaaaatagtaaatcagaagtatttgtcttctattcttggcttctggctagaggtcattaattatttacctacattaagaaataagaataaaattataagaataatagaaatataggtataataatttttaagcatataaagatctttttctaaatatgcattttataattttactgaaaaaataatattgctattttgtggctttttttccagatactttgagaaaattcttacagaaggcatatgaatccaaaattgattatgacaaggtaatttttcattggtgtatttattattaaaaatacgaagtatgagaaaaattttggctgacttgaaagtaaatctttaaataaccagttattcaatctgcaattttagtaatcttttttgactaagaactttcatggaatattgaaccaccttacaaatcccatttgaaccatagcagaaagttcaaaagcggcatttgaagaaaacctttcaagttgtatgttacttaaaaattaaaaactaaggccgggtgtggtggctcacgcctgtaatcccagcactttggtaggctgaggcgggcagatcaccaggtcaggagtctgagaccagcctggccaatatggtgaaactccatccctactaaaaatacaaaaattagccgggcgtggtggcactcgcctgtagtcccaagtattcgggaggctgaggcaggagaatcgcttgaacccaggaggcagaggttgcagtgagccgagatggtgccattgtactccagcctgggtgacagagtgagactccatctcaaaataaataaacaaataaataaataaaaactaaaatttcttcaaaaagaaatattacgctacttttcttttagctgagggatgagaactccctcattaagtagaattgtttgaaaattcctagagatggtgactgtgtactctgttataaaacatcgtttgttaaaaagtagctttgcatttgtataaggtttatgaagctcctgctgtgtgaggacatggctagaaatgaaggaaggtggagataaggatgatagcctttggtctcaagaggctagaagacagatagcagggaccttatctagttaatgtaacaaggatcatacaagggcagagccggtgggatgggtgggcgctcttctcacattccaggcttcagaacatgtgagcaaagaggtcaaggtatgaaagaagcaggcccctggccagagggagagaaaaaaaggtaagttggggcctgatcctgggagggccttgaacgcctctcttgagtgaatccatagtattttcagaaagtatcaaggctccttgcatttttctgcttattacccaagttgctgccaatgcactcctctgcgactgcctacctaaacaggagaatggtctttcgattttggtgtgttatgactgtaggttgcccatgaagcaaacagacaaaagtgtgtagcactgaatgtaaggtaataaaaattagttaaatgtgttggccaggttctcagtccttctcggtaagtaaagcactttgttccttcctttcctggtttgaaggcggttatcgggctaccagtaagcagatgtataggtggacgttttttactggcctgcacaacaaatctgaatgctagtgtttccggcatgggagaaggcagttcccttttgcccagtgaatcgttacctcagatacggtagttacaaatggcctcatcatattcttaacttgcaagaaaattatactcaagagaaaagtaaatgcagaagaggaaagccttatctagaggctgtttacattcagcaagggaagactgtgtggaagatgaactgagctgttcccagggctcacagccttccccagcaccgtggaaagcattttcattcctcagtgccacaggcttcatttatatctcccaatgtgattgtcacgtcggcctgtatctctggggggaagaaaatgacagttgcttttatatttatattcaaaattagcataacatgcttagccagcccgaatgccaaccaaggaatgagagtaggggcagtgcttcagtgggatccgggacttggagaacgaggtcctgagacccttaggtggccttgaggaacctgtgagtctgagctggagctgggctgccaccgtccctggcagggtctttgtgcttgacttggtattagatttggcactcccataaccttggtttatacccctggctcctgctctctttccttgcctttgcaaatgaataaaaactctgggtgggggtgcagtggagagaagatgcagaactgagcccacatgaatgagctgattcctactctagatgtggaactggaccatgctttctaccattatccaaagaaagaggttaattaaacaaaggcaccaaaggatccaggccacatctagaggagggctttctgactagatttagtctgataaaggctgagtgtgttgggttggttccccctgaagcagaccctacaccaatgactccagggcaagtcgtttatttgggaggctcagggaacacagattcctggtcttacccatatcttctgactcatgtgctgtagcagaggagcccaggagtcaatgtttttctttaacacacactgtgaggtgcttctttgtgtctggtgagcttgggagcttgtgccctggttggtaacaagcctgcgtgccccttccccaagaaagggagaccgggatcatttaaaatgccctgtgtgaccccagaggcaggtatatctgaactgccccccagtcccagtattttggatgcctcatttaaaagtgattcccatcacaaatggtgttccctcagagctgtgaagatctgccagctgaggcaatagtttctacacttttagatttcctggaccagaatatttcaaaaaagtgatctagaaaatgacatagagccacctaattttattttgccacgaaaggacatttaaaatcaaacattgagatgtttggaataaaaatggtgggaatccgttgatcgctttaagcatgggaataacaccatcagatttttattttagaaaagggcacttgggaagatgctgccgttggttctggagttctcagatctgattctctattttatgaagatcacatggttagtgtgttaagcatttgtaacttcccctctaattctcctttgcgtaaccgattagcatacggattaagaaccttttgggaaatccaaatgtaaatgcaaatgtaaatttcagaactcagagcaacttaaaacactgtttcttgaattcacactcaagttttatattcctttctcatgtttatccttctatcacccaatttcctccttgtgggatatgaatgttcttggttatggttgatcactcataactttttctgtctgttttcttgatgcatgctgcctctgccaaaattcctcacctgcgttgacttctacagccagaaactgtaatcttaggtctaaaggtaaagtccttgtctgctatttcaagttgctcttggaaagcctttctgttcttcactgtcatcctcactgtgaagatttttaaatgatccgtttcatttctgtttttaccatcacttttaaccttctctttgaagaaagatatctctgtaatatttacatgttgatcttcaacacaatcaacaaagcactggaagatcttggtgtatcaaaaaacagtttaagtagtgacaggccttcgtctagtcggcctagaagtgaactcttgaaatgccgtatcttgttcctgatactgcctgatatttagtcagtaacattagaaaggctgtgttatcaaatgtacaatttgtatttaaagctaaaattagtgcatttaaaacatttcaatatatcttctctcctgggttttggttcagtgtctcctggaattcagatagaatttacatttctttaaaatagtaaacaatttagaatatttgctgagaaatgatggaaaaatacgtaagtaagagtttggaatgtctgggctggtttcttaactgacaagtggtattggtcctcgaggaagggttaagtcccctaataatgcctcttcgtggacatgggatgggggttagcttaagcgtgagcacatacttgatgaacccagcaagattcagccactcttttttttaagcaatttaatcttaatatggaagtagataatattcaagtgccagatgcaccagatttactagtcttgagcatttgctcatagagttaactaccatttattgaatatctactgtgtgctgggcacttggctagttgttttcacattcagtcatttagttatttgtccctcacaatatgtttatgaagttaatgctaatgtgacaccatccactttagggacctggaagccccctgctgggatgtaaagcaggtctctcctccaagacatcacatttgctcccaggaacaagactcgtgttttattttcctttgctcagcttggcttcagaattcgcagctgtctagattgtccatttagttgcaaactgccaacttgcaattttctgccattctttagaaatataagcagctacatgcatgaattggaatgacatcaagtttcatcatctagttgcattaattttcgtttatcccggaataaagtcagcagtgattatgaaaatatgtttaggtcacatgattgtatctagaaattgtgctgttcctaggaactcaggaaccgtctgagacacggactgccaaggtcggtccttcactgtggatctggctcctgggcatggcatgagaggcacctttagtaagtgtctactcagtgcttggtcctttgtcacattatttctaatcatgcgttatctctaattttcacaatgacttgacaaggtacgtattattatttccagtgtatcagaggaggaaccttgtgactggcctgtgtttaacgtgcctgacttagagtctgagccttttttctaaattaagatacattccagattcttgagtaattgtttgccagccaatccaaagtgctttgaatctttaacttctatttttgcagtgtgcaaagcgccttatatgtagattgttatgcaagtactcctgccaagatttattgatccatttaaaaacaaacaaaatacctttggggttaaaattaaaccatttgcagctcaaactagcatttagacatcttcctaggggttgaaagacatcttccagtaaccacttggccatgtatttaattactcaattctctgcttcctctgtttcaaaaaggagtttcaaaatggaaaaaaatacatacatatatatatatatatatgtacacattcagattcaactaatgatggcagataggttaaaaaaatgaaacatggccaggtgcggtggcttacacctgtaatcccagcactttgggaggctgaggcaggtagattgcttgaggccaggagttcaagaccagcctggacaacatggtgaaaccctgtctctactaaaaatacagtactcgggaggctgaggcccaagaatcgcttgaacccaggaggcagaggttgcagtgagccgaaatcgcgccactgcgctccagcctgggcaacagagcgagaccctgtctcaaaaaaaaaaaaaaaaaaaaagaactttgtacaccatggaatgttctacttattattttgtgctacaatatgtgctgtttcttttgtttgaattgcagattgtctactatgaagcagggattattctatgctgtgtcctggggctgctgtttattattctgatgcctctggtggggtatttcttttgtatgtgtcgttgctgtaacaaatgtggtggagaaatgcaccagcgacagaaggaaaatgggcccttcctgaggaaatgctttgcaatctccctgttggtgatttgtataataataaggcaagtacaatattggacaacaactgattgtgttttaggctgatgaagagtgtctcacatttttatggttaaaccatgagtttaaaactaaacaaaatagcaacaatgaaaaaaaaacccaccaaacagaaaagagagagcacaagcgcttgtctagagtgattcgaaaggatctcattatcatcagacattagcgttgtaatgaagctgccccagctgagctgatgagccagccctttggtatttttttcctcttgaaggtatcctgagaaatcttggactgcagaaaggacttacctcccagcacagcagactcctcttaattagctatttgcaggcaggcagcatgcagaaaagagagatggatctgcaagttgatttgctagttgagtccaaaggaatcttgatggactgatgcagtccctgaatttggaaatttgaaaatattgtagaatgcaattgactttctaataataaactcatttttcttttatgtgctgttaatccgctaccctcccttaagtctggaacacacttcacgatttagggtatttctgctgcagtgtctgctgctttcaaaagttttccagttgactggcagatcagaaatacattgcttttaagatgacgttttttcgagtccaaattagattgctcatctgaagatgagtgcgccacagcagtgttcccttgttatgttgcttggttattgttccacatttcctcattcattccgccactgtttcatgcagcagcagaaatcaatgtgcctatgacatgaaggcacagcaagggttggccaacagcattctctttgttgtttctccaaacaccacacgtgctcagtagccgtaagatttttttcaagttttactaaggaagtgatattcaggaattttaacagaaaacagtaagtcctgtgagttggagttctagctgcaaacccaagaaggaaagcctttaagtgtggccaccagcaggggtctgataagatgtgtatggcttagggtgaccactcattggaatgcagtgtacccattgaagaggactgggcagctctggatttgttgataccagtcaccatgacagatgaagtgaaaaagcaggttgcagaccatgtgtagaacatggtaactatgttaaaaaaaaaaaagatcaggaaggagacacaagcactgggtcatatttttatagagcagatgggatgtgaaagggttgacaagaaactggcaacagtggttgcctctggaaaggaaattgagggttgtggaggggaaggaaggaaactcatttcttgctgtagctgggctttttgtctcttttgattatgtaccaggctcgggtattacctattgaattaacttaactgaaacttgaacagtgacaaaaaaaatcttcacaacagacaaaggccaatgcattaccacttactccagggcagtgactatttaagcaaagacatacttttgctcataatagtggaataatcgaccttaactatatatgtgtgtaaacaagatccaagtgaatgtgttagctgacttcatgcattgttggttagcctgagagttacttgttttgtctccatgacattggggtgtgggcatctctgcctgtcaatctctctctccctctctctctctcttttcttcccttttctgtctctccttgcctctctgtatttttctgtcactgtctgtctctttctctgtgtctccaaaaatcatttgatccttgctatttttttcacatttgattttgagaggcagtttgggatcaggtcagtagtggtcattccaaatgtccctgtctctctctgctttgtatttgatatactctaaggaatgcacagactcctagttataggggtagttacctctttttcagctaccagaagaatctcggttacagccctgaaacctgtcaaacccaccagggatccctggtgtgtttcctgtaagcctgaaacttacacgccacatttgtctagacacttgcacagccatgtttacccgctccatacaccctggacctcaaagatgagactcggatgtgaaagcatcatctgaaaatattgcccttagggcttctagttagaatgattacttatttaacaagttaataattttcacttgatgtcaatacatatagtataatttcaggaaaattctttattgaatgtagttatttatttacctgaataagtgtttacatgtgtaataatcggtttccaatatattgggtttctttgtttttttgagacagaatctcgctgtgtcacccaggttggagtgcagtggcgctatcacagctcactccaacctccgcctcccgggttcaagtcattcttgtgcctcagccttcctagtagctgggattacaagcatgcgccaccaggcccggcagatttttgtatttttagtagagacggggtttcaccatgttggccaggctggtctcaaactcctggcctcaaatgatctgccctcctcgacctcccaaagtgctaggattacaggtgtgagccaccgtgcctagccactacaatgggtttctttttcaaaattgaacttttatttgcattacatatagttatgtgttatgcatgttatagttcactctcagctgattgagttatttacattatgttgaaaatgccaaaatatatcaacaataaagctacaaagtaaaaaataaacttggcttccatttatatataaaatgaaagctaccgttttgttaattcttgagaactcagacctatcaaatcaatatatgttttttctaagtatattttaatttagaaaattctttttaaaaatgcatgctgtgattctataccctcctacacattcagttaaaacaatttttttccccacccaagtaaaacattaatgtgaaaattgtaattttagatttcaatataattaaaccaaaattgatcaactgagattgtgacaaaactaaaactaatttgatttcatatagcacattgaatttatgtataaattataaataggcttaaataagagtagtgtgtgaatttttttttctcactttaaattctgatgagaatttaagcttctttgccttgaactttttgatttacggtagctattttaatggcttccttttgaaacccccactggtttgcaaataccttaaacatcatgagttgcataatacctacatattgtttgcaagtaatccaagctaggcaggaggattaaagacctggggaaagttaagatttctctattatgagtagttagacaggagaagaaagaatgtcaacaaaatcagaagattagataaaaataagaaggacaagaatgtcaacaaaattggaaggcagaaagggaaagaaaggaagtggagggaaagataggagagtggactctctcttgtttaatgcagggcctctagctgacattgacttcagcggagatgggacactattgagaaataatctttgcatgtgatttttaaaaaattctttcagaaacccagagcagtccacctgccttttattggactcaatttcattttgtggctcaaaatattacaattaaatgcagttggttttcaagttagttttgagcatccttttttattttaaaaaatgtaaatcttttctttagggggtgaagggaagacataggtgttaaggagaaagatgccctagcagatgtgcaatctacgtgggcatgttctttctctgagggttcccagaggcagctgtggaatgtgagaattccgtagacactgacatatgtttatgcagtctgtttgaccatcagcacaagatcgaatttccacccctcactgtagactgttaagcagatgtgtcttactccagaaaaagccaactttcttctcctttctttctttttgcttgtcaccgctgcatttctcataaaggttatggaaggaaaaaaaaaaaccatgacttgacttcctttgtatgtttctaagtttctgagtatgataagccagtcttgctaataattcccatggtgaattgaattccagtcattttcaggaagctggctattttcaaagttcattttatactaggacgtgattgccaattcctgaatgggaggctacaaggttgtggaaaaaagattgagctagatgtagcagtcctgaaaatcatgaaactttcttcatgtgaatgaggtctggttacttacagtttaaatgtaatttttctgttttttaataagagagctgttatagactgaaagcatttgtttggctttctttggctaattttattaaatcagtagaaattggtaagagcaatacagttacaagttttaaagtaaaggtttaaaagtaaggtttagccttactctgtaaaaagcttgtaagtgtatatgtgagaaaattgttttttccataataaaatatggaagatgtaaggccataaaaataaaaattcctatcttctgctaaaaattctaagagattttgttgtactgcttaaaagcatatgttttagattgtttaaaggcatttatgtcaaatgccagagaactgatattcactgaaatatgatttcctttttgtgttaggtaaatgtctcttggtatcattgtattctaataattgactggaggttgttcaaataaaaatatttattcacataccttttttttgctaaaaaatacactgaacaacagggagtgtctaaatcaatacctcaaagcataatttctttgttctttatttgtgtttgaatagaaggtttaaatttgagattgcacatggagaccatcagttggtctgcatagtatttaaattttttttgttagttgctaatatttaaaaattgggatatttcacataagaatccagattcccagatttttgtggataatgggaagatacggtaatatttatctgccatcatggtggtgcttttgagacctgcagatttagctgctcctccagcccatttttgatctcccagctcccatgaccccctgctacccaatttttctttaatgctttctgcctgcttacacaggcattagaatttaaatacaaaatttactttgtgcattagtttgaaaatcagctttgaaaatcactaacttctactctataattatacttagtagagattttttttctttttctctttaataatggggtcttgctatattgcccatgctggacttggaactcctgggctcaagtgattatcccatgtcggtgtcccaagtagctaggattacaggcgtgtgccaccacacccagcttagtacagtttttttaaaaaattatcaatgctttaattttaattagatctattatcgattagtagaaatgtaggtctaaaatatgtagcattgaaatcgagtgttgtagtgaaatgtattgcaattcatttcactgactgatgatcttgtgagtaaggacatttattcaataaaggcacaaagacagtttctgactcacacttagacatacggccagtcttctaaatagcccaaatgaaaccaaagcttgacccctaaactcaccttcctggagcacccggtgtagacaagactgcattatacaaagttaacaaagaagtagtttccacctctttttaggaaaattaaacatagataagaataaagacacattagagtctgaagtgagacatatcccatcagaattaaagggagcctctgggagttcaaggcaaggaggttcttctccctcttggggatgggtggaggggtcagtgagggtcaagtgaaagtggacttggactggtgcttttctcccaagttcactttgacttcttgtatctggaatctgacgagtgaataaaataatctgggcatgctgggaagagagagcaagagtccccattgtgaacatcaggtatgttcaggtggtgagcctgacttgtcatggtctagcatgttttgaatttggcatggcagcaagctccaggtgcattgctgactcaacttttgtgtgtgtgtgtgtgtgtgtgtgtgtttcttcttcacaaaactccaaaatcattattttgagtcatttgacctctttggcatgagtgcctgatttcataactcttattgctgtagtgcattgactcccaaaataccaatgcaaatttaggacaaattataaggcggtcattccaaatgggacagaatattgttaatatttattttataagattaccatcagaattaacccaaacttaaaaatcttagagaacgtgtacatccagtactactctcctttacagcttgagaagcacttatctaatctaagttagactggatctaatttatctaattacacctaaagatctaaactgatcttttcagtttggaggtaccaactggagtccacagaaagaaagtgattttctttggcttgtacagtacggtggtggcaggacaggacccagaacccaaatcttgtgcccctagtctagtattcagtacactgcaacttgttgaccataggaaatatccctcaacgttatattacataaacttttttggagcattttgggaaacctaatagacctatgttatactgcaaaggaaaagtatgttttgaaaccagtaatgaaccaatgctccttggaatgtatttatactataagtggggtgaataactgtccccatttacccaggatggagaaatgggacatgggattttaggtacaaaaaatgggatgtttagtcatcctacccgtaattatatgcacgataaagcctcaagacattgcttttgctgaagaggcaatgtattgattttctgcatctgatatgtttttaaagtttgcctattatttgcaaaactttagttctaaaaccatatctacatcttccactggatgattacaaactaacaagttttttacttgcttctgcaggtaaaaatagttcggctatgtttatgtagttccttactgccatctagtggccaaattcaacagaaccttggcaattagcaccttctaatggaacgttttttgccacaaaaacaggccaattggtcaaattattttaagtcaaaggattattagtaactagtcaacagtaagagcccttaaaaattattacattagacattttagatggcttgaaaaaagacgttttggttttttcacatagagaaggctttcctgtaagatgcctttcatatggcgtttttgcagctttttttgaagtatatttcacatggaggaaagtgtccttttcacaagtacatatgcgtaagtgtccatgaagtgaatccacccacgaacagagtactctgccattctacccctgctctgggcataaccactgacttccagcaccaccgtttcacttgtttttgcactttctgtagatgaaataaaacataggtgctctgttgtatgtggcttctcacataccattttaatgctaaagtaactcatgttactttccaacctggacaatacaggcactaacagacaattagagaaaccaaaagccttagaagcccaagtactcttaaccattttgatgtttttttctatagccttctttggggggtcagggagcagaagtgtgtgtgtgtgtgtgtgtgcgtgtgtgtgtgttcacacacatgcatatgttcacacaggagggtgtgtacacacattcacatgaggacaggaatgggactggatcatgcatgctgccttataacctttttgcacctatttcctccacaatttgttgtgaacattgttccattgacgagagtcattttgaacagaatggaaatttgcctcctctgagaggactgagtcctgttttgtagcccctacgtgggtggtggtttgactgtgggatggcggtgggaaggatgtgggaggagagggcagcgggagaggaaaggccagtccttctgcgggctcctccctgactcctgctctgctgcctggacctggacaaagaaccatggaccacacagtaaatgctctttcttctctctgccttttcctgcagcattggcatcttctatggttttgtggcaaatcaccaggtaagaacccggatcaaaaggagtcggaaactggcagatagcaatttcaaggacttgcgaactctcttgaatgaaactccagaggtaaaaaccatcatctctatgtaccgcgatgctttatatttttggttttttggagttcctgtgtatatttggaatctcaatcaaaccttctagaaacacagtgaatcagcgtagactggtgttatctgttaatgtcctggatgaataaataaagccctggaaagtgattttcaagagtacaaaagccgaatttcagaattggtgtttttattttattgagatataacttaacattacagtacatattatagaaggacttaacacttttaaggttcttatgattttttaaaaaaatgaaagtttttgggccttattatcctttctttttggcagtatagttaactttccatcacttgtacaaagttactcttgtaatgaaaccatccagctttataaacatccatttgctaatgtctgcatagatatatcgaattccttgggaaggacacacaagcagtctctatcacacacacacacatacacacacacacgtacacatactcacggacacacaaattgtctttagagaagggcaatgggatggttgaaggttggggatgggagaggaagtaccctttcttctgtattccagaagtatgttgatgaattacctattcagaacatcgttttaaaaagtgtctgggcaggaagcagcctaaatgtgtgatgctcatgggttccaacttgtcagaaagcagaaaggtttctaggaacttgctggttacctgaaaatgtcctgattcaggaaaacttttgtatattaaatctctttttgccctcttattgcctttggaccttagaaggggaggtttcacagaatgttcttttttcagcaaatcaaatatatattggcccagtacaacactaccaaggacaaggcgttcacagatctgaacagtgagtaaaatttaaagttgcttcacattttttttctttgttttgactcatctcccataaaacatggactaagaaatgacgtacgtgaaagttgggggaagacagaaagatgattccctagccttattgccaatgtgtcatttgttactcagcgattagaatctagccctcatcgaaggcatctgtttcacaggcagcaggaccttttgttttttcattttacacttactggaaagctttagcattcttgcagtcacaagctctcattctccatttccttcgtgagggaaagattgttttcctgccaggtgccctgcacagacgcagccagctcctcagagaaaccggtggcttcagaaatggctggatggagagggctcaaccctgcatgccacggtttagcttagtgcttctcaactgggggttgatgtttggcacatttggtgacatctggagacattttgggttgtcacaatgaggtgaaggtggggtggggacagtgctaccaacatctagcgtgtcaaagccagggatgtctctaagcatcccgtaatgctcacaacagcccccaccagagtgcagtccaggtgcgaactctggtctagccgaagaggttagatacacagagagttacacacaggtcaagatgataacaccatgctcctggtgagtctgtagtgattaatttttcactggggaacagttggtgcggagaccctgaagagaaatctccatacacgagtttaacccctagacagggtccaggatgcaaatgggttttgcagcaggaggggcttgaggcagtgtggagagaatgagggaggggtggagagatgagcagaggccaccctcaccatctttgtgcttatttgcatgtaggtatcaattcagtgctaggaggcggaattcttgaccgactgagacccaacatcatccctgttcttgatgagattaagtccatggcaacaggtaagcagtgggcaaaaatgaccaaagaaagttccatccaggagtccctgggcttggcaggcttgctcactcttttcttgttccctgagagctggcttggggagagccaccgtccgtggccatgcagacagttcaggtcaagacactgtgttgcaatagcttggtggtgctctttattaaagcttgtgccaaagctgttgccatttcagatgatagggcggaatttgttaggggcccaccacctgttttttaaacacagttttatcggaacacagccatacgcatttgtttagatatcatctgtggccgttttcgtgctatgatggcagagttgagtcgttggaagagacatagtgtaacccacaaaacctaaaatatttaccgtctatctggccttttatagaaaaatttgttgaccctgatacagaagacgaggaacctatagacatttaggtcaatcagagggaaattgattcctttttccttcctgaaagagaaacttagaagcaagaaatatacatatatatcttaagcctccatggtcaatggtataagtgacagatatactgattttaataaactgcatgctccactgtaatgaaaaaagacctcctaggcccttcctaatgttgagaaaaatgtccttgctaggtacttgatagagaggacaattacatctatgtaaaacactcattttgccaatatgaattttactttcatgagctagcatccctgagaacatagtccataaagaagagaattgcgtggaaattggaaaattattccagttccttttggcatgccttatagttcaacaactgagacaaatgtgcagaaactggaggacgtgcagaacaggccaggagttgttaagatgagtcctaactaattgaaaggcctttccttccctttctccttgaccctgcaaagggtccagccaacaacaacattctcgacatcgacaataacatcacgaacaacatcctctgttgttgatggcttagatttctctagacatctgttctcttcattaccaaccattttctttccaggcaaggaaacggaggcccagagaagggatatgacctcagagaccattgcctcctgccttcctgtcctggctttttccacgtggcttctccattccttctttctttccttcctccctccctccctccctttctttctttccctccttcccttcttccctccttccctccttccttcctttctccttccctctctcccctactcctttcttccttgcacacctggcccctgtccatcattccagtctcatttcctgtctgggacactcatctctgatgtcccttcctcttcttgtaagaacatcagttctgttggattagggacccacccttatgaccttatttaacgttagttacctccctaaacttcacatcagtggttagggcttcaacatacgaatctgggggagacatacttcagtccatagcaattgggtagctgggggaagtggtggagttgggattcagaccccgttctagcaagagcccgtgttacctggaccaccctagatgggacttagtaccttgaagagtgttaagatggcgggtgttgtggtgacatccacaaggaggcaacacatgcacctctggagggagaattggaaatctgggattccagtgctcacatggccccagatgatgccaggcaacactgcagcctggatccctcgtgcgtcagtgcgtttgcaatattgaacaagatctttttgttttccaaaagcttcaaagtattttatgtctataacatattaactgggttgtaactctgggagtcattccaggcccaccaagctcacatatattatgtgactgcaatgttgaagagatcacatatattatgttcgcaatattgaagagatccaaggtctcttccagccccaaattaagtaacagtgaaggctgggcaaaagtatatgaatgatggactgtggttgatgcatacctttactttttgcccaatgttcttgcaatttttagtaactgaattttagtactttttaggtaaaagataactcaaagattctctttttctcttcagtgcctcgtgtgttattattaaagttctttttttttgtttttttaattcagctggaataagttaaaatttagcctctggtgttgccagaacttcttaaaacatgcttaataaactactccagcattttttttttttttttggctaaaaatgatttgcagccctgtgtgtttagtgggttttggagagagcagaaacatgcaattattttggtgtgcaaatcatatttggactgcagatcaggacctataaaggaaatatactatgggctggcaaaatggtaattaagtaaaattaagaaccttagatatcttttccacttacttcagatacataacatatatgatcctggtgggcctggactgacttccagagttacaacccagttaatagtttatagacataaaatactttgaagcttttggaaaacatgtatttattacttctgagtcctccgtgtgacatgacagaatattatccatgggctagaatggctatgaacagtgcttaatacacatttcctgggccatgcatttattttatcacctgtcattctctgttgttgttggtggtggtggtggtggttcttgccttaagataacttatcctgaagtcaaagtgaaaagggccgtgaggagtaaaccacagaacattaaaacatgaagtgtgttttagcctgctcttttgtgttcttctgtgtatttctgagtatttctgtgtatttctggtgtatttctgtgtatttctggttgcagctgagttcttttctgttttgctgtggttaatatttattgagcttctgctatgatccagataccagacttcatgcttcaccgacaatactggatgataggtgggagagatgaaaaactgatacccagggtgctcttcgatagtcctaaatattattacaaccttaaatattgttaagctatgattagactgaaattcaagagaaaatactgtttgaagcagagtttaaggttttctattcaggtaaagtgatagaaatttgagaccaaggaggatacagttcaattctggaagcctccccaggtctctgggtacatcctcaaagctaggttgcagtatctaaccagacctagtgagcatgggggagaccatctcctaagagctcagccccacaagctattagggcacaacctggaatcagactcttctagtaaccaggaggtgacccaacacactccctaatttatagatgagaccaaggtcctgagaagtgagagtttgccagatgtgctgtagcagggtagaagcagaactgggatgcaacctcgggctacttttggctagactcaacacggaaacatttttaaaatgtgtgtgtgtgtgtgtgtgtgtgtgtatttttaacactcaagtattttctgttatgtggctgccaggaaagggtggggaagccagtttttaagggcattcattcttttccttgaaacacccaattgcagcttgatcctccaccatctgttctgtgaaccacagagcgaagacctgaccaagaagatttatttgcttagaagaagagcaaagccctctaatttaaatttcacatcttttactgctgtggctttaagccactgtagaaaaaaagttgtaattccctacctccctagtcatgtataggtagctccatcatccagatagatcaggatagcaaacaatttttctgggtttcctggtactagatgcccctccaaatacacgatgggggttacatcccaataaacccatggtgaactgaaaacattgtttaagtggaaaatgcttttaatacactgtagaatatgcgttgtttgccctcttgattgcatggctgactgagagctgtggctcgctgccactgcaagagagtaccctaccacatatcactagcctgggaaagttcaaaattcaaaattcaaaatttgaagtacgattttaactgaatgcatatggctttccactaacataaagttgagaaatcataaattgaactgttgttagttgggtactctctgtatgagaatgtgtcctagggggtagggtttgcaaaattaaacagaccactgcctttgaggcattttcatctggccgatctaacccccatgttgcatgatgaatgtggtggtgtttcagtacttgaagatgaggaaattggctcagagtggttcaggagcctatttttagtcacacagcaaatagcaaagctgagatccatgcctagacctgcctgaatctaaaaccgtgctcttaactactgtgctagttttgattccaactcttagaataaatagagctaaaatatatctgggcgtaatccattttgatgtgcatttacactgctgggattgacttgtttggactgagctcttcgaggctgggtggtgacaactttgctggccactgctggccagttaatgctgcattgaggtgtccccatcatgctcctgggttggtgcttgccccctgcttcttgttggaaagagaccccaagtggaagagtggagctagttggagcagctgttagagcagcctagcgatgctcctgtattcccctctcagactgccccttgcagtgtgtccctctcactgccatctaaagtcatttaccagtccatcccaagtccctttagacacacttttgtagatgaggagggacttggcatgtgtgttctgaagcgggctgtgtttccatagcgatcaaggagaccaaagaggcgttggagaacatgaacagcaccttgaagagcttgcaccaacaaagtacacagcttagcagcagtctgaccagcgtgaaaactagcctgcggtcatctctcaatgaccctctgtgcttggtgcatccatcaagtgaaacctgcaacagcatcagattgtctctaagccagctgaatagcaaccctgaactgaggcaggtgagcaggcatgagcttgccatggtcaagggctgcatgctggggattgccgggcagggctaagcgtgggtgacctatccagtgcatggtggcctgacttggagaacagttagcccctggcctctatcagccttcttgagtcatttgatcaccatattggaatgaccacagcaggagcaaaggagtaaaagggaaccgcttcatgagagttctctgccattgaatgaaacatatcagaattaattcaactttacaaagaaaaaaggaaacagaatatattcatgtagacctttaagaattaaaaaatttttaaatatgtgcctagtctattagccatgtatgctgaaatagaagaaaattcgattttcccaaagaaaagattttttaaaaagaatttggaacaacttggattacatgaagttctgagatataggtaaaatgaccatgtctattaatgatgtctaactgatttagttagatacacacctcacttttttttttgagatggagtctcactctgtttcccaggctggagtgcagtggcacgatctcagctgactgcaacctccaacctcttgtgttcaagtgattctcttgcctcagcctcctgagtagctgagattacaggcacccgccaccatgcctggctaattttcgtatttttagtagagacagggttacgccatgttggccaggctggtctcaaactcctgacctcaagtgatctgcctgccttagcctcccaaagttctgggattacaggcatgagccaccacgcccggacacctcacattttatataaaataacattttagtatagatacatttttcttacttttacatttcattccattgtttttcagacatttcacctgcgacatctgtgtctggatatcactataatcatttttgattgaacagttttttagagcttagttgcaactgagttgtttgagatagatccttttggggtgcactttttatttttaatatgtatcaaacatgtggaaaacttgcaagatgaacgcatggatccctgttcacaccctttggccacattcttcaagggtcacgattttgcaccatttgctttatcatattttatcagtttatctgtctgttcatatcatttaatatatacatgcataaaataatatgcattttgttcttttctcaaaaggtaagttggggataagttgtagacatcatcatgcctttttacccctaaatattttattgtccttcctaactttacataatcattatataatgatcaaaattaggataacattgacccgacactaccatttaatccacagtgtgcattcaaatgttgatctttccagaaatgccttttataactatcttctctttttccagtctcaggtccaggccaggatcacacattgtcatttattgtcatgtctctttcattcctgttaatctggaacagtttctaagtatgccttttcttttgtgacacagacatttttgaacaggccatttactttgtagaatggtcttcagtttagggttgtttgatgttttcttgggattaaagttactcatatttgccagggaactatcacagtagtgatattatgttcttggacatcatgtgggaggcatatgatgttcgtcccagtattggtaggagctagagaacttcaaatccatatataatactgtaagtggaaaaacgattccagattactcactaatataaccactgactacattgcttttaaaagtgactttaaaaaggttgtttacaattttattggaccatctttagtatttgaagttgtgaagtcatatatattacatttgtgttacatttctttgcctcttttttttagctggttacagcaagagacctccagacatcaaaaaattagtgcttgtcaaggagggtctgagctgtgtttttccgaaataaaactctattgttcaaaagaaactgcgattgtaccctgtagtatagatgctttgggacttggaatatacaatgacttctttcttcatgaggaattgttttgaaggtaacaaccttgtcttatattttgttttataaattttcagcttccacccgtggatgcagaacttgacaacgttaataacgttcttaggacagatttggatggcctggtccaacaggtatgcttttggaaaaaatacaatcactgattttatatcattttaaagggtggactagtacataagaaaataggaaatagcaaaaaagcagtgaaattctagagaaaggttgccaagaagggtgatagctattaccttccaggcaattgtgttcatccttccagttcccagaatttttgttaagcatctatgatgtgtgaagtggcatgctaagaccatgagagatcaagaagaacatgacctctgctttcctaggcctcagaatttagtgggtgtgatgttgaaccccacactgattctgatacaaaatgctgtccccctctgccagtgagtcagctatactggttttctttttgctcttacccaccttcacgcatttgcacatgctcttccctctgcttggcactctcttcttttcccctctccaagtagctagctacttagttgtttttttgttttgttttgttttgttttttgaggtggaatccccctctgtcacccaggatggagtacagtggtacgatctcggctcactgcaacctctgcctcccgagttcaagcgattctcctgcctcagcctgttgagtagctgggatcacagctgtgcgccaacatgcctggctgagttttgtatttttagtggagatgaggtttcaccatgttggccaggctggcctcgaactcctgacctcaagtgatctgccccccttggccttccaaagtgctgggattacaggcatgagccaccacacccagccgctacttagttttttaagcttcatatatcaccttcttcttctttttttttttttttttttttgagatggagtcttgcgctgttgcccaggctggagtgcagtggcatgatcttaggtcactgcaacctctgcctcccaggttcaagcgattctcatgcctcagcctcctgagtagctgggataacaggcacacgccaccacaccctgctaatttttgtatttttagtagagatggggttttgccatgttggccaggctggtctcgaattcctgacctcaggtgacccacccgcctcggcctcccaaagtgctgggattacaggcatgagccgtcacgcccggtccatatatcaccttcttaacagacctttcctgggcttcttgactaggtcaaaccctttttaatacattcctgcagcattgcaaatattctctgttaatttcctcataactgtaattatttctttaatgtctcccttcccaccactgaactgtaaacccccaaaaacgcagggacagcattggtctggaattccctgatttccccagtagcttaccaaatcttgctctttattcaactaatagatactgagtatgcaataaatatttctttttagattatggttcagtagagtgcctgggcctatgggaaaatgagagaatagttttattcttaactctgtcttttaggatggatgtaaaaagtctcaaatctctattaacccatttatgcctagtgttctattattggaacgctaagcttgtgggagttatttatatcctactgccaaatgtcatcatcaggttctgatttttcacaaaaaatatttgcaaccttcagcataaatggattaaacacttgtctacatgtgtttaccagacacttacttaccgacaaagtaagtggtcccttaaacaagttattccaaatttcttggtctttgcactctggtgttacttcatttgttaaagaaggcattgaaccgggagatctgtaagacaccttccagctgtgaaaccccccatgacatctgcaagtccagacacagatgaatgtcaaggagtcaggcatatagaaaaccaacacaagcaccggtgtgctgagtagcttagtctccttggaggaaacggaagtcatggatgttcattttgtgacatctgatgtagctaggctaaggtggaaactggctggcaccataagtcttatgaaggctgaaagtgctgtacttttccctggcagacggctaagtacaccagcagacagtgaagaaacacccactaaacctcttccagaggaaaggctgcacgtttagttgaattatttggaaataatgatatttccctctcatggggggtttagccccaaagccctatcgctcggcacggttgttttttgtttttgttttatcctcaccacagttgtatcctctgtaagccatgctttaggacatttttttttttcgtttagactatatttctaattttatttttaattagaaagtaagattctgtttttagaaattcactttctgtctgcccatgggatagtgtaccgatcagtgtctcttaacctcagcactggtgacactgtgcatggctgttagcggcgggggctctcctgtgcatggttgggtatttattagcatccctggcatctccccacacaatcccagcagcaccccacaactgtgataaccaaaatgtctccagactttgccaaacataccctagttgagaacaaaatttctcccagttgagaaccactggtatagatgtatgtacccataatagtacctaggctctgtgtaaatatgttgattttaaaattatggggaccccctatatgaaaaacttctaacacgacctttgattgatataatttaaagacaatgctctctaaaggggatttttttagactcttgatgaccatataatgatcatttcacccttaagatgaatgttatatttttattttgcctctctactcgtactgagttgcaaaatgagtgactagtcaactagtctttgacttaactgtgtacatccttttattttgaaatatttttcagggctatcaatcccttaatgatatacctgacagagtacaacgccaaaccacgactgtcgtagcaggtgagattcacagtttgaggtgattttgtgattggtaggtcagtacagagaaatgtgttgccaagtattgcattgttcttttcctcttaaaaaaacagtttctcattaaagctgtcaaaactaaacaaatatcagagctttacaattgtgtcattcggacagttaaaaacaagaacaaacctgttattgaacactgataaacatcatccagcccagccagcagctgccacatggttgacagccacagtgtgatccaggtccccagagtgtgcgccttcagatcttacagcagccacagtttggaaactcattctgattttcactgaagagaaaaactgtagattattatattttctaaacaaacttaagatcaaagtttgttgtttttttttttttttttttttgagacagagtctcgctctgtcgctcaggctggaatgcagtggcacaatctcggctcactgcaagctccgcctcccgggttcacgccattctcctgcctcagcctcctgagtagctgggactacaggtgcccgccaccacacccggctaatttttttgtatttttagtagagatggggtttcactgtgttagccaggatggtctcgatctcctgacctcatgatctacctgcctcggcctcccaaagtgctgggattataggcgtgagccaccgtgcctggccaaaatcaaactttttaaaggaatactgtgtaaaagaatttattttgtctattaggaagattttagtctgtccactgatgctgaggcagtagtattttaggctccttaggttgcttacaattttggagagacagaatcaatgtagagaggattctacccaaattactgaatcatgctgtatttgggttatgggatttggggtgaacgtacagagggagagaatgcagtggaagcagttctttgtgcaaggttagagacaaacctggattatttgcttggagaagaaatctctgggctgggcgcagtggctcacgcctgcaatcccagcactttgggaggccaaggcgggtaggtcacctgaggtcaggagttcgagatcagcctggccaacatggtgagaccccatctctactaaaaatacaaaaattagccaggcatggttgtgcatccctgtaaccccagctactcgggaggctgaggcaggagaatcacttgaacccaggaggaggaggttgcagtgagccgagatcgtgccactgcactccagcctgggcgactctctctcaaaaaaaaacaaaaacaaacaaacaaaaaagaacagaaaagaaaagaaatctctgaagctatgtaaagctatcttctatttcttttaaaaatcattttagtgctgcttttaactgttgagtaagtggttttaaaatattatgttagagatacttgattatttttccctaatagtgttcttttgacccatgaagagtttgagtagaaaagtgttttatcttgctacatgacactttagatgttttgtgggtttccttttgcataatataagattctacttgaaaatattaagatattaaggacagggttcagactttgatgtgctaacctctgaaggctgatgcatgctgtatgtctgacagccacctgctcactaatctctgtgtttaattttttggtttcccctctctggacctctcctgcccatacatgcagcccttccttgtaagtctgacacagcaaattgtgctgcgtgcattgagtgcggcccatggtgcatggtgccattgcctgagcacaagccagtgtcttcctagagccgggccagtgccgctctgtcagcctggatagcaggtgttcagtttcatgtcctttgctctcgtgtttaggttgtctcatcccaacctgaacaaattatttagtgccaactctgtgccaggctctgccaggcactgggctgtgagatgcataagccatgagttctgcttgtcctttagccagccatgtgtacacagacttgttgtcacactctggcaccatgaccaggtttgtgtcagggaaacaaggtgaggagaggctagtgggaggctgcctggaaaaggggacatcgagagaggtttgaagagggtatatgccaagagaggacaccagccacatagagagttgtcagagcagcaaagtcacggagccatgtcgtgcgtgagggcaagccaggctgagaagcaggcaggggccctaggagctgctcctagaacatgggaaggctgcattccccatctctgaaaatctcagaagtaaccaagagacccactgagtcatttttggccaaggatcctcaggtgttttcacacacgagccatttggagaaggcaatgagcccccaaggagtaacagtcctttcttaacgagtcacacccaaacacctgacactctgttgctcgcttctgtgtctttgatttctagatgtcatagctatgtgaaaaacaaactttaaaaaagtttccagggccgggtgtggtggctcactgctataatcccagcactttgggaggctgaggcaggaggatatcttgagcccaggagcaacatggtgagattccatcttcccatctctacaaaaagtaaaatcataaaaaattagctgaacatggtggtacacacctgtagtcccagctacttgggaggctgaggtgggaggatccttgagcccaggaggttgagcctgcagtgagccatgatcgtgccactgcactccagcctgggcaatacagagagatcctgtctcaaaaagaaaacaaaaagtttcttgccatttttatgtgattacgttttggaccaagctaccattctgttctctacctgaagttcaccgggagcctcaggataatattttgagaactgctaatcttgtctctctgctgctgcaaagagatttacctctcttgcaggatcagtggacttttaactgattagtgtgttcttagtatgtccgtgtcaagaatcacttggatacagccaccttcagaactcatgtgagcgttggttcaggtgaaatcctgcacccaaggttagttgattttaggacctgttgtgatggacactctccagaaaactggttttctttgaacttaactcctgtctgagaaattacgactgagcaaaagaaactggccactgagactactattggtttttgcatagaaaagacacagatgtacttcctaagatgtctcaatttgtatcagtcaggtaaaattttaacaaaaatatcacccagagtcacttgtgtttttctgtttgtttgtttgttttttaaatgggccttgagaattactctgttcctgaagactttttttttttttttttttttttgagagcgagtcttgctatgtcacccaggctggagtacaatgaatggcacaatcttggctcactgaaacctctgcctcccaggttcaagtgattcttctgcctcagcctcccgagttacaggcatgcgccaccacagctgggtaatttttgtatttttagtagagatggggttttgccatgttggtcaggctggtctcgaacttctgacctcaggtgatcctcccacctcagactcccaaagtgctgggattacaggtgtgagccactgtgcccggccctgaataccatttcttagagggagaaattattattattatttattttcccaaaagcatgtaagggggacgttagagtcagatacacccgaatttcaatcccgttaccagagctttctttaaaagacccatgggcaattaattgaatctctgtgagcctccgggtcgctgttagcaaaatggggaatgagaatatctacctcagtgaaataaatgagttactatttataaaagtgcctggcacgtagtctgcacaccatgcattttaatcacttgatctactttaagtaaagtaagtggaagaaggaaactggcttgacttcagtaccatctcattaaagtcagtgctcacagcttgctgttttgttaagtaattgcttttcgatggtcttggctatattcatgcttgttgaagaatcttaccatattttaaacacatacatgacatgagaaaaactaaaggtggttcaggcttttctgtttggttccaaaacaaagtttctatttcctccatgtttgcaaatgcataacaaaggtatctttgttttcaggtatcaaaagggtcttgaattccattggttcagatatcgacaatgtaactcagcgtcttcctattcaggatatactctcagcattctctgtttatgttaataacactgaaagttacatccacagaaatttacctacattggaagagtatgattcatactggtgagtgtctccttggagctggtgaactacgagagtgaactttgtagagaacgagataaaaatctgacattagcaccaggataatcttatgagagcaaattgtacaaggaggccatgacctgatatatgttaaccccagtttatgttcattagaatttgaagtgctcgcttagaccaggcagcacatatactaaaattggaacaatgcatagaaggttcgcatgactcctgtgtacagatgatatgcaaatttgtaaagcagtccacgtttatttttaagttgaagactggaccttcgtgagccaaatacttgataatttgcaattttgatgtcattagtggtttctggaatactagttataattatcaaaccaggcatgtaatgagattttaaagcttaaaagtaattaagaaggacatctgggaaatattcttttctattttataaaccaatataaacattggtttataaaataaatatgcagaggttggttcttactgaaagagcattcttttcttagctgtgccttgaaaccagtgaattgctcccagagcttaggtgagtggctgtggtgcccactggttgtcccattagaaaatgcgagtggtggggtttcacccggggtgagggtggtggacatcccagtcagagccctggggagggaaggacttggaggctcaagaaatttagaggatcatgtattaggtaagatgcacttctctctctgaagagctcagcaaccctttatatctcccaagcccatggggagtccggagactcattctcatgtggattatttcttatacaagaccacaggatacgtaattaaaattatagcagttgctaccatttatgggacagtttcagtgtgcccaccaagcactttctgcacatttcttttaatcctcacaaaaatcccctggcctgtgttccatttacaaccctgttttctagacaaggagacgggctcagggaagtgaagtgacttccttaaggccagctagcatacggcagaggtaggttttgaagctccatctggctctgctttttgtcatcgccccacaaacaacgagctccagggagcacctgccatgattcccttgtggggctaggccaccccagcaagtctcagaacaaacagaaggcagaccctttctcagtgaacttataatctcattagagatcagagcttcacaaacgagaaagaatagtaccattacctgtctacctgactgccatccatctcccttccttttccccctgccctgacactatgaacttatcttaaaaagaaaattaactaagcagatgctcctttaactctaaaagttttctgaggccagcctccatggcttatttaattttgtgccctcactgtctggaacaaacctccccagaatggctgcctgtggctattactataattactgagattgctcttaattgaatgaaaagcttcagtggtgctgcttggtcaagaggcaccaagcagaagtaaattctttgttttttagtgtcttaaatgttgggaagcattactacccttgaatgcctcttgctgccgggaaaggttgttctcccttccgggaatgccagtaaggcctaattgaggatccattatcctaaatgtctcgcaggaccattatgaaattgagatttgcattctgcaggcatttgatttaggatgctcataaaattctaaataccccatgcatctgccatgtaatctggttcagtaaagaacacagtggccccaaatatttctgcaaccacaaaatattttagaaaaaggcccagggaaaagaatccagtatttctttatctgtagtctaatcatgatctagccatatgccttcattattataataggcacttagaacatcagaggccttaaaagaacataaaaatcttaagggtgttttcatcagtagccttggagaagctcagtgtgctgtgcttatgaaccctgtgtgacacccgtgggaatcaaagcaaaggaaggaggcaatgatcccatgaccaccaggaacaatgcaaacctagaatcaaaataaaagtttctccgctggttgaattggaaggcaaagggtgtgaagggatcttttcactcaacattattgtcctctgaggaattgatagaagactctgattttagaagccaagaataactcatcggctccagccttagtccagcagcttttgtgttagggtggcgtcacctgtgtcatgtatactaacactgtgcttgcctctccaggtggctgggtggcctggtcatctgctctctgctgaccctcatcgtgattttttactacctgggcttactgtgtggcgtgtgcggctatgacaggcatgccaccccgaccacccgaggctgtgtctccaacaccggaggcgtcttcctcatggtgtgagtgtctgctcctctctgtcatgtgggagtgggaatgcaggagagagactgggtgctgactctggtgttacatgaacccggcgccctctgggccacgtacgcggggttccagctcccaggtcgcgaggaggagagatcctgctgtgatgggaccttccttgatttgggtacttctgtcaagcctaaacattaaaaacaaacaaacaacaacaacaacaacactaaattacttgcattttgatagaccagcttaagcacactatttatatatggcaggctccttttaaaagcagctagacttgctttcttgatttctattgttccttctttttgtcattcattcattcattcatttattcactcaccagaggctgagccaatctcttgtgggacagcagtattggtcagcagtattgctagcttgaaattctagaaacttgatttagaactaaggctgccaaggcagaacccttaggatttagacaggtagcctagattaggcgtgagaacaggtgtgaggcagcagggagaggtatgcctgcggcagaagactggagcgactgtccctgcttcaaagacattgacctccaagttagtgtctttgaaaaacctgtgactctatggatacgacaaagcatagcaggcatgagccaggtgcaaccggggccagagccagaaatgtaagctaggatgtggtgtaatggctttgaatgctctgcagggagagagctgtgtgggttctaagtcaatattctctttttgataagatcatgaagaatcagcagtagggagtgagatggggctctaggtcagtggttaaggtttgaaacaattcctttggggagtgaagaaccaaaacctgagtcgtgttctctcatttatccacttaagtcactagcacggggctgtgagtgagcgggctggagagcagatgctgtacattaatttgattaatttgttttttaaaactttgggtcttcaaaccaaattttcacacacacacacacacacacacacacacacacacacacacacacaccaaacaaacaaaaaactttgggtcactggaacaataaatcaggcttcttacaagctatgattataaatcctttccctacaataattaggctccagtatggtataggcctttacaaactagagtcgtaaaatcaccttagagctaatgacctgaggtctaccagggctggacaaagaactccacacaaacagaagcttagatgctcacaggccaaagcttaagaaattatatttgtctgggcacgatggctcacgcctgtaatcccaacactttgggaggccgagacaggcagatcacttgaggccaggagttcgagaccagcctgaccaacatggtgaaaccccatgtctactaaaaatacaaaaattagctgggcatgatggtgcacacctgtaatcccaggtacttgggaggctgaggcacgagaatcacctgaacccaggaggcaaaggttacagtgagccgagatcgtgccaactgcactccaacctgggcgacagagtgagactctgtcaaaaaaaaaaaaaaaaaagagagagcgagaaataaagaaaaaaagaaagaggaaggaaggaaggaaggaaggaagggagagagagagggggagggagagagagagagagggagagagagaggaaggaaggaaggaaggaaggaaggaagaaaaagaaagaaagaaagaaagaaagattagctgcaagtacctgtggacagagaagaaaaaaaggagaaatcacatctgcaaggaaaaatgaatttgtcttcccttcttgttcaaatgtacacagatacacaaaaatacattaaacagaagattattaggttgctgtaaaagtaattgcagtttttgccattacttttaatgtttaattgattttaagtttaatgttacaaatttaacaacaatttaacttggttgtttgttagtgttttggtggggtgtgtatatatacctatatttccatcttttttaactacttgaaaggtagggtaaattgctctgaaaaatggtgcacccctcatcgaaattttattcacactttttaaagggatacaacaaaaaatgtgatatcaagcttcaaagaaagaaaaaataaaacagagaaatcatcaacaaattgatggaggctaaaagctaccttagtttagatatactaattctatagacatggagacctgtggcatggaggccataggattcatctcagcagcaggaagaaggctggtaactgtcagatgttgtttgattcatttcaaggagtctgcagacttagggtggctatgatttctttattgttgaatgcaaccagtcagcatggcacgctttgctgtgcttggcagaattctctgcttcttgaggtatctggtaaacaactggataattgcaaaaggaggagatgatacaagcgtcatcagtctaatcagactaattagaagaatgcagtttctttttttaaaactgtttacttgcttgtagaaaccagaaagagtatgtgtagttttgttgcaaccaaaacaccacctgctctttaaaaatggtgacaccatccaatcaagagcatggtctctttcaaaaccaagcaggaattcttacctcccaaaaggataccagtgaaagatctggaaggaggaaagtcagagagccctggctctgctctgatcttttatcttcagatggtcccagaagcattttattttaattttatttttgtggagacagtcttgctttgttgcccagattggtcttgaattcctggtctcaagagatcctccgacctcagcctcccaatgtgttgggattacaggtgtgagccgctacccctggccccaggagcattttagcagctgaataggatgcagtggtgaatgtattatctgttgcaggggggcgtaggaggcctctccagagataaggagatctaacagaggcacaagcatctcgtgatttgaagcccatcataaaacttcccattttaggtattgacaacaatgccaagagcagccacaggtatcacaaactgggcacattcctatggctggtggcagtttctgaggttatggaaagcagtttgtcaggctatatagcaagggctgtaaaaagaccatgatgctctttgccgaataatttccattttggggaatctaggtgaaggaagtaagccaaactgtgaagaaatcatcatgaagagggaggtgttttgcagtattagttatgatagtgaagcactggaaacagcctacatgtccagctgtagaagagtggccaagtgagttatgacacatccatgcgtggaagatcacatggcccctgaaatgataactaggtgacagtcctaatttgttgacaaaagtttctaagataatgcatttgaaagggggtcagaaaatgatgcatgtattatcctaactgtgcaaatcaggggttagtcagcaagcttttcccaagaagggccaagtaataaatattttaggttctgctggccatacacagtctttgtcgcatattcttgttctttttcaccttcttttgctctttcatcttactcttattctccccctcctcttcctccttttcaatattttcagtacacattagccattatctcactaaccttagaaaaacagaccttgggcagtggttcccctacccctgatctaggaatgtgcatatttagaaaataaaggctggagatagtgactgtatgcgatttagagtaagagtcatggtgccctttttagacagctgtcttggtttatatgagttgtttaactccaacacacagacacacatgctcagacagggatcctggcatggccctcactcacttcttctttgtgctctcccagactcagaagccttgtaggtcagagaaagtcaggctgacctaggcctttccaatgaacattacttgctatttattttagtgaagatacacaaatattgtaattatggctttggcatctgcaggcaaaggggagaatcgtgcccttaggaggggatgctgtggtgattgcgagcatcagcccacacccttctgaggctccttcctagtgtgcaccagtgatggtatttgatttggcagctgcacctagatcgatttaagagtttgttctctatgtctacatctagacagctaagcactgctagagaaaagctacgcaccgggtacattgggtatccctacaaacttaggatctcctgtctcaaatggacttgccatagtctgacaaatcttttcttttatttcttttgttttcttccttctctttctgttttttttctttttcgacaccccccacactttcctgcaacagtcttttcactctgatacgtccattttcctctacttcacccctctctcagcagatggctttacctccttcttctcagagaaaatagaagccatcatgcaacttcccatcatcaaacttccaaacacacctgctctcaaacacatccatcccttctctgcagtgccagaagcagagggattcctgcctgctcaggttgatcccgccagctatattctcaatcccattcctttccctgatcctgataaaatggatcatcccctttcttttccatgtctttattctcttcctgtctccttgactttcactcaatacttaaccaggctctgttctctcccttcttcagaaataaaagcagaaactctttacccacactgcctcttccctatagctctccttcccccacacaggctctcagaggagtgatctgcgcttgctgcctgtgccctcctctcctgctgactctccggtccactccgatgtggtttcctcccccatgactgcacttgtttcaagttggcagtgacctctctgtgcaatgcagacagcacattggttggtgacagccgaccattccttcctcctgcaaacactccttttgtcccctaggtggcatcacacccacatagttaccttcctacttctctgcctcatcatcttggtctggtttgctggcccctccttccaccacccatctttgtaaatgttgaagtcttccagacccagccctaggctgtttctctctccaagccagccctggggataaatgaccatggctaagctgctggctccctaaggtacatccgaaacccagatatctgagctccaaacatactgactcctacacagtctttccattcagtgtctctggcaacaagaccaacatccagcccatcttctctttccttccagagcatcagcgctccctggcactgcccttggagcccttccttgcctctacctctcgtgttcagcgctttccgagtcctgctgcctcctatattatgcctcctgtgtctcctatattacatctccatcatccaccaactctccaccaatcctgcccccttaggccaccctcatgggccatgataacagcctctgcaatgatgtcccatccacgttggcacctgtccagtctatcctttacactgtgccaagagtgcttgcgttcagagagaagtcgggttgtgctcctcgtgtgctttcagccccttattggctacccctcgtgggtccccaggcccctatacagtacatccccgccttccctggagctgtccctccccaggtccccaggcccctatccagtgatcccccgcctgctctggagctgcccctccccaggtccccaggcccccatccagtgtacccttgcctgtcctggagctgcccctctccaggtccccaagccctgtgtcactcctaggtggatgatctgtttcacctgaataacactgaagaccccagggctattttatagagtataggtagatttagtaagtatcagtaggtaatattgaatcattctttgttgaattactaactttatatttttttccagtggagttggattaagtttcctcttttgctggatattgatgatcattgtggttcttacctttgtctttggtgcaaatgtggaaaaactgatctgtgaaccttacacgagcaaggaattattccgggtaaatatgattttctacccattatgaaaggacttgaacagaaacttggatttcttcatatattgtgcattactttaagagcagtgcaaacttataagcagtttggtttaaattcagtaaatttcttgaaattattaaattcaataaattttcaaatattcaataaattcagtagatcctttctatttgtaagagaaaaaaaaatatgggagcgtccttattttctgatcataagcgaaatcagggtcaggagttaaagtcctgccgctcaatgtggattgcagtatttggggagaaaagaattttcctcagcagtagtttccaaatgtggctgggaacatcagaagatcagttcccaggttcctcccacacaccctgggagattcccaggtgacagaggtgtccacacagatgagccctatatttgaagggaaagagactgataagcggatgactcgtggcagagggctgacctggcacacgtctagtagtgccaccaggaaatgaattcaagcttcgctttctccctctcagtccaactcccaccgactccgggggaccagttggcagttttgctttcttcattaatgaggaaattttgttgtaagtgtctggcttctaattgtggcttttctgtgttttttttttctccagattctgatttttaaaatatatttctatttcactctttcattgtatatacatttttccgcctaagatctgttatgaaatcaggagaatactgttgtcacagtattttagttatttcactggtcgagggagattaagttgagatcaccagcaggtcaaatgatctccatttggccttttgataaaagaaagacatcaagccaggcatcattatctcctgtagagtgtcgaatcattcaaagataatttttcttaagtacgggatatttcaaaaccatttctacttctcaagcgcgtacagtcatgcattgatttgcccatcttaaatatgtatgatgcagaggcagagagtgtgtgaatttttttttttttttaagacggagtctcactctgtcacccaggctggagtgcagtggtgccatctaggctcactgccagctccgcctcccggtttcatgccattctcctgcctcagcctcccgagtagctggaactacaggtgcccgccaccacgcgcggctaattttttgtatttttagtagagacggggtttcaccacgttagccaggatggtctcgatctcctgacctcgtgatctgcccaccttggcctcccaaagtgctgggattacaggcgtgagccacagcgcccggccaagtgtgtgaattttaatacattttgtccagagagagaaatagcgcagcactgggtccaccacttaatggctgtatattagctattctgatcaaatggtctactctgcttctatttataatgtgtttgttttattaagataaaggttaaagattaaagagttctcggcgtggtggctcacgcctgtaatcccagcactttgggatgctgaggtgggtggatcacctgaggtcaggaattcaagatcagcctggccaacatggtgaaatgctgtttctactagaaatgccatttctactaaaactacaaaactagccaggcagtggtggtgcctgtagtcctaactactcgggaggctgagactggagaatcattttaacccggaagggggaggttgcagtgagctgagatcgcgccactgcgctccagcctgggcaacagagcaagactctgtctcaaaaaaaaaaagttctcaaaaaaagtaattttaataattttgattgatgctacccatcaataaatataataaattgatttattccaattatactattattttctagttataagttggaaatcaaccagaaaaataatgttatgaaaaatttccaaaatacattcaacagaatattaattttattatattagtatgtatgttagtgtaaaactgatttttcgaaaatacttcctaatgtgttctaggttttggatacaccctacttactaaatgaagactgggaatactatctctctgggaagctatttaataaatcaaaaatgaagctcacttttgaacaagtttacaggtatcaatatttgcattatacatttcgctatttaagatgttctttttctggaatgagaattttgcatttgaagtgtttttaaatcttaattatatgactttcatgtttcagagataagctgtcctgtattattttaaagacacagtagaaattagatcaaaaatatattccttctactgaaagcctttgagaccttggaatggtgatgtttcaaggtctcgctctccaataatctctggaactcactagttaaggtgaccctacgatgcactaatgggaccccatgagagatcttgttcaagaaccacattgctctaagtaaaagccatccaacaagaatagtgaacaggtgattcagtgatttggatcactgatagggggaaggcaatgcagttggagagctgtctgctgtcaggactcctgggctctgaccccggccccctcctggaggctctgtgccctgggcaattccacatcttctctgttccacgggggtcatgatcataggaatctgaccctggggttgttttagggttaagtaagatacatgagattcgaatggtgcctgacacatcatttggacttgataaacaatacctgttcttaatattgttcatctaggctggccatagtggctcatgcctataatcccagcactttgggaggctgaggcaggtggatcacctgaggtcaggagttcgagaccagactgaccaatatggtgaaatcccatctctactaaaaatgaaatacaaaaatgagccagtcatggtggcatgtgcctgtagactcagctactcaggaggctgagacaagagaattgcttgaaccagggaggcgcaggttgcagtgagccaagatcgtgccactgcactccagccgggcaaaagagtgagactttctctcaggaaaaaagaaaacaaaaaaactatgtatatatatatataacaactcctgacctcaggtgatgtatactaccagccatcagactaggagcccatttcaccactgggcaaaagcagacactacacagtgagctaagagaaatgcatttcatatatatatatatatatatgtttgcatttcatatatatatatatatgaatgaacgaatatatatttcatatatatatatgaacgaaatgcatttcatatatatatatatgtttcattgaaatgcatttcttttagctcactgtgtagtgtctgcttttgctcagtggtgaaatgggctcctagtctgatggttggtagtcttaggtgtctgatcttaggtgtgaacccatgggcagctctctgctatgagccacagggaagggagtcactttaagagtatttgaaatcagttcacaggatgctttttaaacactggtgacacttatttctcccactgtgagaccgaagggccggaggatgtgtgtgttaacatggtgtgttcatagtgaatgaaactaaaaataacagggcgatagtgactcacattgagagcagaacacttcataagtcagagacacagaagcccttccatgccctacacgtgaaccaccattcttagtgttcttattattctctcagagttcctttatgcaattttaagcaagtattaaccctatgcctagcgttattatatagcttagtagtattcgccatcatttgatattcaagagacttcttaagcattttgtgaatttgttatacttttaggtattgcaaggttctttttgtactttttaaaaatattttgaaatatgcacacatttaattaatcaatggtcattagttcctgtagccaaaccacagaagttattttaaaacaaatgtagctgtgtcattactaagtatttatttatttatttatttatttatttattttctgagatggagtctcgctctatcacccgggctggagtgtaatggcaccatctcagctcactgcaacctctgcctcctgggttcaagcgattctcctgcctcagcctcctgagtagctgggattacaggtgtctgtcaccacgcctggctaatttttgtatttttagtagagatggggtttcaccatgttggtcaggctggtctcgaactcttgacctcaggtgatccacccaccttggcctcccaaagtgttgggattacaggcgtgagccccaagcccggccattactgtttatttctattacctaataaatattatgctaagtggtaatagtggtaatttaacaaaatgctaagagctagcacctgcatggtgctttctgtatgccaagactgatggaaacacctcggttgtgtaaacttctttaatatccacaccactctgtgagctaggtaccatactaactacttttattattttatgagcacctgttgtaaagcgagctaagtaccatactaacaactgcttttattattatatgagcacctgtggtaacggtatacctgagatattatacaaaattgaattagactctggtcctacttgtcaaggaaaacctagcagggagttagatctgtaaagaagtgttctagaatacagtgatacttacaggtgacagaaatgtattgaggctactctagaagccccaaggagggtgtcttatgcagtgtgggaatggagggattaggaaggactacctgagggaaatggcacctaagctgatcttagtaggttagtggggattggctgggccaagaaggaaaaaggtacctcggccgagggacacagacaaaggcagggaggtaggggcagtaggctactctttgggagcttttaaaaatcccactaccactttggggccaaatacaaagttaaaggacagagaaaagtcctcgtttccattattcagcccatggcagtgaaaactgagcttctggagccctgagacaacactcagaatgaacttataaatgttactcgtaggctttgtcttctaaatctttcttaaatcttcttcttcttcctcttcctcctcctcttcttctcctcctcctcctcctttttcttctttttcttcttcttttcctcctcctcctccttcttcttcttcgtcttcttcgtcttcttcttctttttctctctctctctctccttccaactggtctccttgttcccattccatcctccaaacctccaagcatgatgtgatgtgacccaaccacatgtgtcacttcctacctaagtttctccattgcttctggcagaggcagctaacaagtgtccagtgcccatcccccatactcagggtagacattgatagtcaaccgaggtcttctgcttagcccagggagcctcaggctccttctggacagcgtgctctaggcatacattagcaatccattggcttttctctgtacagttaaacccgttcaccacccctaggctgagaggacaaagccaagtgtctcagcctggcatgctagctccttgtgattctggctcctgcagagccctccagttttacctctggttgttcatgcatcaggctctaagttccaacaacaccaacccgcttgttctctggagggaaggatcacacctcctagggatgtgggaaagttgcttcttccaggcatccctcaggcctagagggccctggctgctgtggcaaggaagagatgccacattcctggatgccgctatttttgtggcaggaatacacatccaatgaggcctacccttcttagctcagtcaagtctaaaaaagtcaaattttcctattttagagatatgattgaggcatggtgaggtgaggccatttcacccagggggctgacttaggttacagccaaactgtgctgaccatcgagatgtctgcgtctcctccctatctctgtcctcaccatgaggatatacgtctaagcgtatatttagtcagtgccgctcggccctggaaatacagaggagaaatgttcttggcaggagcttcatagaatcatttaatgaagttcataatctactctgcagtgcgtctctttctagcaagtattgtttttctttttaatgagaatttatttcttcccaatctgtaaacttgtggtccacagcagaactagagcatgtaggagacagatggaaagatatattcatcaatcctattgcatctcttccaagaacaggactgttagtaaagcaaggcaagaagtcagaagtggtgggtgctgggggatttgtgtggccactggggacatgcagccccacttctgtgttcacagattaaacaaggagaaacagtgagcctttctcctcatcttcagtggtggacagaggtagctgcaacagcttttctaggtcctaatttctctgttcattcccacagtgactgcaaaaaaaatagaggcacttacggcactcttcacctgcagaacagcttcaatatcagtgaacatctcaacattaatgaggtaattagaactcgtcatggaatcgacataacacattccttcaccaaatctaggtgtctttctttcttttctggaaattgagatgaaatttgcaaaagataaaattaactatttaaaagtgatgtttggtatatttacatggctgtgtaaccttcacctctatttggttccaaaacgttttctcactgcccgaccagttgtctttcttgtggtttgagttttcctagtggattatgcaagagcctctagaggggtctcactgtggacacttcacagggaaagatgtcattttgctgagtatagacgtttttgtgggaacaatccctatttgttcctcaggaaggctctcactccaagaccagcctccctttctgccagtctctgaaattgggcactcagtgtggaggagcatggtccataccgttctccttccttatgagtcgcaaggtggccccaggactccagtgtgaactaaagcaaaggatggaagccgacagaacctcctcttttcttttgccagagcccacaccccagtcttgagtcttaagatttggaggaaagaccattccagggcacactggcctgaagaaagagcaagagtttcagctgcccaggcctagggtgccgttaacctagaccaaccccactccccgtacctggaggcctcttctgactttctgccagaaagaatctagactgtcatctctcttttgggtttccagaatgagagcagaaacaaagggatcccaagggaagaggctattcccaagcccccacagcttgcgcactgctgctagagttaccatgaggccatgatggcttgatggttttcaaatgtgaaaagctgtgtttgaaagctgtatatgaaatgtttattatcttccctaaatagtccatacatatacattaaacagacataattttgtaaccaaaccctaaatagatgcccgtcgtgctgtgggtcacttctctgtgtgctatggtggatggcagttggagaggttgcctgggacgtgcaggcactccatgctgctggaaactgctgactatcaatacaaccctgaatcaccaaccgtgtttccataatctttgcccaagtaactaaacactgttccccaaatgatatctcccatttcttcctactcccagctgttgtgtgtactggtcccttccctggaatgccagcctaccctgtgtccctttcagcttctctgtttgtcaaagctggaggcttagaagccatgggacccatagagaaagagatgggaaatgaacaggatgggaagtaccaggaagagatgccagtcaagtgcttgcacattttttttgtcttttgtggtttgattgctgtttattttttatttattttttaaatttatgtttaaaattttttttaaatagagacaaggtctctttatgttgccctggctggttttgaactcctgagctcaagcgatcctcccacctcagcctcccaaagtgctaggattgcaggcatgagccaccacatccagcttttattgctcttttaaagcatttggtttgtgacattcttggagaataagggactgatttgtaacttccttttttcgcagcatactggaagcataagcagtgaattggaaagtctgaaggtaaatcttaatatctttctgttgggtgcagcaggaagaaaaaaccttcaggattttgctgcttgtggaatagacagaatgaattatgacagctacttggctcaggtatggcgcatgtgttcatgcttgatcctttaggagaaattaaagtgtcagatgtgtatttctgttttgcttttgattttttattaagatgagattcacatgatgtaagactattttaaagtgagcaattcggtggcattgagtacattcacagtgctgcacaactacttctgtagttcctgtagttccaagacattttcatcaccctaaagcaaaccccaaagcagttgcccccatttcctcctccccaaacccttggcaaccacccaaccacccatctgtggtctttttttttttttttttttttttttttgagacggagtctcgctctgtcaccaggctggagtgcagtggcgcaatctcggctcactgcaacctccacttcctgggttcaagtgattctcctgcctcagcctcccgagtggctgggactacaggcgcctgccaccacgcccagctaactttttttgtatttttagtagagacggggtttcaccatgtttgccaggatagtctcgatcccttgaccctgtgatctgcccacctcggcctcccaaagtgctggaattacaggcgtgagcctctgtgcccggcccatctgtggtctctttagatggatttacctagtctgggtagtaaaaaaaaaaaaaaagattacgtaacacataatcttttgtgtctggcttctttcactgagcatgtttttgctgttcatccatgttataacattatcagcacttcattccttttcatagttgaataatatgattgaataaaaatatggctttatatgtataaataagaatgcaaatgttgccacctgtttaagagtttctgacctcacatgattactttatggttatgaatccatgtttaatctgttgcctactagatgttgtgttaaggcttaaagatcccatatccataagacaatttttttcacttctattgtagactggtaaatcccccgcaggagtgaatcttttatcatttgcatatgatctagaagcaaaagcaaacagtttggtgagttcaaatcgtccggttaaatactgtagtagttgtagatgtcaaatgtcagtaccattcttagggaagctgctggagaggttcctcacactgtgagtaatcccttgttcataaacacctctcttgtgtgtgtcattacgtttccgtccacagccattgccatgtgtcccaaacccctctgagccttgaatgttgccaggatggtgtcgggttgatgcagggcaggtgaaacctgctttattgaggcagtggcagggttacagctcagagactgctcttctggaggggggctaccccataggcagtgtgcagaaagtagcagcctagggacagttttgtagtcttatttataaccgctttcaatggaatgctaattaagtggcaggttgttcagaaatagctagaaaatgagtggtaacttcccagtgttgccatggcaatggttagctgtcatggcgctggcgggcatgccttatggtgatggacagggagaggtgctttgggctcctctttctggtttccgccagtcttcagtctggtccggagtcccacctgccttctatctcaagatgactgcagccttccaccctctgctctgagtgcgggcaccctcactactttagctctggatctgtttccacctctgtcaacccctccacttctaaacacacccgcccatgcacacaggttgttttcctgctgtgtagggaatagtacctggggctgtgggaggaagatttcatgtgttgctttgagttaccaatagtaagtgatgagggaaggcagaggtaccaccccagccccataagcttcccctttcaactcctctgctggttggtgactacttggccttaactggccccgtccaagaagcagcacgccatggtgacgaggaacctgggctcttgaagaagacctgggtctaagtcttgactcggtataggagcttaggcaagttatttctcccttctgccttagtttcctcatctgtaaaatggggtaatggtatctaccttataggattgttattcattaataaattaaacattcattgagtgtgggttaaataaaaatttctctatgggtccttggttttctcacctgtaaaatgggaaactgagagtggctgtatcatggtgaggtggagagaacacagtctctggcactcaggaagggctcttgggtgtgggttattgctgagagtccatggttctgtgctttgaaggagggtgtcttggcactatccctcacaccagcccccatgcccactcacagccatcattgcatgcttatgtcatggcagccctcctggctacacagggcccctgaggatagcgagagtgctttgagtctggaaagatggtattgatctttgttttttattctttttgtagcccccaggaaatttgaggaactccctgaaaagagatgcacaaactattaaaacaattcaccagcaacgagtccttcctatagaacaatcactggtaacagtcaacgacgtattgtatttcactgtgacctgagcaaaatctactgcagctagttggctttcatttagcctcacgacacatgctcatctctcataggtcccagctgactggcttcagccccagaagttgagctggcctttagacaaaataattgcacctccctctgctgcttattcccttccgtttttcatttgagtgtgaacagttagataaaatctgtggctgtctcttccaccttgctctagtttccattgctgtgagcaggccctcctatgccccgcatttagctacaatgctgtggactcacttgattctttttctccgagctttgtctagaaatatgtgaaggtgaggttaagtgcttctctgtgtagatccacttagccctgtctgctgtctcgatgggcgttgcttcgtctctcctctcttccatcctttccatttgcttctcaccaccttctggcttcttttcttaatgcaataaaggcagtttctaacaaagaaagaatgtgggctttggagttagacagacctggttttaaattctgcttctggctctccaaactattgtagcttctcagtttcctcatctaaaaatgttgggatcaataactgccttgctcttacctgttcatggaaatgaaggacctgacctcagaggatctcagtaaagaggaggaattattatattatgcttgtgtgtgggccgtatttgtagccatcaaggagagaagtgtgtggaaactgggttactgaatcagcttgctcaggtttggacaagcaagtaaatccacttagccccaacatccaaacatccgaacatcccaaccactaaacttcccagaactaatttccaatactctttttttctctctcgatttcctaatttattagattattcacaaagtcctgtcactgaattttcttaatttgtgtccagtgctatttacaattttctgtaactggtttatgcctaacacaaatttgttaacattttaagctggttttgcatttttttaaatgcttttttcccaagtacacaggaccctttggccttttcagagctagggcaacctgtccacaaataggcagagtgcttcctacctgagacctatgatctaaaccaggggttggcaaactagggaccctgcaaactacagcctgcaggctaaattcagcctaccagcagtttttgtaaataaaattgtattggaatatggccacacccactcatttgcaaactctctgtggctgcttttgattacaaggacaaagttgagtagttgggactatggcagataaaacctaaagtgtttactctggtgttttacagataaagtttgtcaacctcctgtctaaaccattcaggttaaccactcttgcggggcaggctctgctttaaataatgccttccactttgatcaaacctaattcaaagggatttcttggatgcctcacatgtgatttgctgttcttcacagctttaggatatgtatccaatgttttcttcaggttttcttacaccagaggcatagaaagtacacattgttggccaggcgcggtggctcacacctgtaatcccagcactttgggaggccgaggcgggtggatcaccaggtcaggagatcgagaccatcttggctaacacggtgaaaccccgtctctactaaaaatacaaaaaattagccgggcgtggtggaggacgcctgtagtcccagctactcaggaggctgaggcaggagaatggcgtgaacccaggaggcggagcttgcagtgagctgagatcgtgccactgcactccagcctgggtgacagagcgagactccatctcaaaaaaaaaaaaaaaaagaaagtacacattgttaattgtgttggttattttttctagaatgaaaccattacagtgtaaagacagatttgatggctatcttgtgggaagggaggacacaaggtatgtgatcttgctgcttcatgtaataattttggaaatattatctgtttcttgccagagcactctataccaaagcgtcaagatacttcaacgcacagggaatggattgttggtaagggtttactttgttttctgtcatggggttttgttataaaataccacacaaagaatgctagaaatgtggatttttgtaggtactttaagttgaaaaaaaaagcctcagttatctacctattttaaatagcaaattgggctttcctactatttcctttctacaagtacttctctaagtgcagacctcatcttagcaggctattagatacataggtgaaatctatgttttgatgtgtaggatgcaagcttggaaggtacattggtccctttcacagagataatgctgcctaacacacaatcccaaacctcagtgccttctaagtataaatgttcattttccctgctggtttgacagcaggcttctctacacatcttcccattgagagacccggcctgaaggagcagctcctgtctggggcacctcatgtcctcatagtggaggacaggagttcaggggatgagtggaggcacaggttgtcctttaagctcagaatgggcaccctgccccttcaccagcgttccattggccaaagcaagtcacatggtcacacacatcgtcgtctggctggggaagtatactccacgctctgggaagccatccaaggagcatatcaataatcctgaacagctaatgcaatctacctaccacaggggccatgtgtgggctgcaagcattccctcagcagacacagagctgcaaggctgggttgtattcatcttgtatctacagtaccaatgggcaactcagaagatattttttcattgaacggaatagtctgtacatgacaggttttgggtatcagccccacaggataaaactaaatgttcctccttccacttttctcagtgagaccaacatcatggctcagctcctttgaagatgtttagatttactctgctccaggaccctcgctatgcctctgatcacagttgccatttcataggtgtgtgtctcatgccaggggaacctggaaacacaccagtgactacgtgcaggtgctttgctacctctgtgaacagggcgagggtgtgcagtggtgcaaggcctttcatgaggggcccagccaagggggttagtcgagggcagaaactggccattgactgagttgccaggctctgtgccctgaggtccctgggcagaaaaggccccatttccaatttgcaccaagctgccatgtgggttggcagaggactcgtctttgcttgtgttgtagtgcaaagactatgggttttgtagtgaaaaaaaaaaaagttcggtgcaaatcccagcattgtcgtatgtttccccagcatggtgaccgtgaagaggctgagctgcctcatttgtgaagggaacctaacaaacagtaccttccttcctgcctcgttgtgagtttccagcaagtccagcaaggccattcctatcaaggactagcccagtgtctgggctagagtaaatgctcactttcagctcatctccttccctgccctcaggggcctgggtgggttctgtcttcctcttaactagcaggtccaggtcgtggtgttgcagagctgagttacagtacttgaatcaatatgcaaattgtctaattaaaactttctatgacttgataccctgataactgtggctgtgtctttcaggagagagtaactaggattctagcttctctggattttgctcagaacttcatcacaaacaatacttcctctgttattattgaggtgagccttcttttttaaagtaagcgtgtccataagtgggcacttgaatgttacagattaaatatttataatgtttaagtatgctgtcatcattttaatgcttctactcatcttttgtttctttccaggaaactaagaagtatgggagaacaataataggatattttgaacattatctgcagtggatcgagttctctgtaagtagtttatcttttatgaatgttaaggggggtcaagtttcaggatagccattttctagggtaaaatctggaagaaataaaattaaatttcccataatttggtaggaagtaagtgaaaggacattgatgtgcaggaccaagacttctggggcttcccaccaggtcagaggacagccacctcctggtcccctttgctcacctttgccatatgcagttcttccatttgcttcccaccccctttttcaggtaacaaaggggccaggtaacaaatttgaactaaaacactatgctgggcacgagaactttttaattctctcagagtcctgtaggctgggtctcactgttccaaaacaaatgaggaaactggaaaccgaggctcagagagaataaggatcttgctttaaaatatatacaattgtccctcagtatccatgggttattcgttccagagtcccctgcagataccaaaatccttgggtgctcaagtcccttatataaaagatatagttatttgcacataacctatgcacatcctcctgtgtgctttaaatctctctagattacttataatacctaatacaatgtacatgctatgtaaatagttggtatactgtattaaaaaaagtctgtacatgttcagtaaaggtacaactatccattgttttctgaatatgttttaatctatgattggttgaatccatggatgcagaattcagggatatggagggccgactgtaactgtatatgcagaactcatccatgcacatcagccgtcagttccatgtcttagagcagcagtccccagcctttttggcaccagggactggtttagtggaagacagtttttccacaggccgggggtggaggtagtttcaggatgattcaagggcattacatttatagtgcactttatttctattattaatacattgtaatatataatgaaataattatgcaattcaccacagtgtagaatcagtgggagtaatgggagacagtgacagatcatcaggcattagattctcataaggagcacaattgagattcctcgcatgtgcagttccacaatagggttcatgctcctatgagaatctaatgctgctgctgctctgacaggaggtggagctcaggcagtaacataagagatgaggagcagctgtaaatacaggtgaagctttgcttgctcacccgcccctcacttcctgctgtggagcccagttcctaacaggctgtggaccggtactagctggtatccatctgtgacccaggagttggggacccctgtcttagaggacatggagatcctttttgtgacaccaagtcaaaagtaaatggcacgtctcattcctttgggtttttgtggatgcagctcaaagtttccctgtgtttccacccccctagatcagtgagaaagtggcatcgtgcaaacctgtggccaccgctctagatactgctgttgatgtctttctgtgtagctacattatcgaccccttggtaagatttcatctttcaaaagaagacacaatgaatccatcatctttggttgtttttctggattatgatatacattataatatttgttttctgcttgagagaatccaaaacctaaaatgtaatattttgatcagtgtattgagatgctaatagactagaaaagtcaaagttctgggcggttgtcttctttcctcactaggattatatttatgagtcaggagacctgcagggagaaaactcctgttagttcagaataatcagaaagacagtcacgggtgtgcagatttctcatcttcaaaagaatactcagtacaactacatttacaaccccaaagttgcatcaaatttcatttgtcattaaaaagcccttagaaatgtgactattgggatggtaagttccagttagtcaggcttaatcctcagctctgttccctcctcaacagatgctgactctctctgtgagtggatcctctccctctgctgaaagcacctcagtggcttcccattgctatctaaagatcaggattcctcatagcccatactaggctctgcatggctgagcctccccattccagatctcactgtgtgctctctgcctgcccgcctttctttctgtgctccagacatactcatcttctttcacttcatcaaataaatctcaatttccccaccttagggcccatgcacatgccgtttcttctgcctggactggagatgggcacacgccgcctttctctttctctctcctcttccgcttcccacctacccactccccactttaagacctaactcctccaggtctggtaacacaatggtggagaacatcaactctgaatgaaaattccacttctacattttcagaactctgtgactttgaaaaattcctgaaattttctgagttcctcgcctacatgggctgttttgatgattcagtgagaggtcacatgtgaagcccttcacgctgttggaacatcagcactcaatgaatattcactactattgttgttccgccttcaggtcttagctcaaacatgtcttttttccaggaagccttttctgatccccaaaactaggctgagctccgttttcctcattcctgtagaactctatgcttttcctttagagatgttattgctgtttcaattacatatttatatgtttctttgattaagacctgtctctccagcacactctagactctccactccatgagtcaccagtgcattcaaggctgtgtccctgttgtctggcataatacctggcacagagtatcactgatgcattgtcaggtagctctctggcacagttcatccctattgccctgtctgtccacctccctcgttcacttgtccatctgtctgctggagggtctttcatgcctgagtgaaaaggattctgcctccgacccaaccacgcagaccctaggccctgcttcctggatgcttccttcttcttgcaactacttttcttgtcctgtgcttggtggcccctgccgccagctttggctgaagccttcattaggggagtccagagccaacctgcttgattctgaattgacgtctctctatgtccagcctccaccacccaccccattcagtcgaaccacatagccaccagatgcctttccacagatcaccagggacagaggggaggcctagaggctgctcaggaagatcataggctggagctgtgtagcagagggtttacatcgtaaggaagagctctgggcatccatttagtcaatacgatagactgtgggctagctaacatgtttattttgtatctcaattgtccccaaaaggattgaggcagcttccagaaaaaggataaggttgttataaagttataaagcttctcaagacaaggttgaaaacagaaatctaacagaacatagagggaagggggaacaaattgactggaagttaggagccaagaggtggtgccacctctgtaattctgaactcagccaggagcaaagcagaaatgcagggtcaagctgtgagggtgaagcaggtgttatttccaagtggcatttggcctcatggtgggctggggagagtcgttctagtgttactgacagcattattctaaggcagggatattgtcagactcgaacttacgtaggaagttggcaggttgtagatttacatagcaggccaagtgtaggatgctggccagagtggcattcagcccgtgacatctatacattgtgtgtttgtcctgattggtccataaggttacatcatttggaatatcaccttgcagatgtgagtggtgtagactgtgtcaccactgaggacaaatgtccccttgaaggaagaaccacttccagactcttgctttgtgggaataagggttcaatgttaagagatggaactttagaattttcaagaaaggatgtaaatccaggtttacatgtgaaatctgacttaaaaatgttggcaactatttaaaaaaggatactttaacattgctctttagacaaaacatggtcatctaccttctatataccttcaagattggttaaattaaacgtgaaacattgattgagaccctagcatgcagtgaacattgtttctaacttcttgacacagtaatgtcatggaatgaagatgcaagcatctcagctgctcagatgcagaacaaagccagaagggttaatttgtcatcttttttttttttcttttttttgagatagagtctcgttctgttgcccaggctggagtgcagtggtgcaatctcggttcactgcaacctccgcctcctgggttcacgtgattctcctgcctcagcctcccgagtagctgggattacaggcacacaccaccatgcctggctaattttttgtctatttttagtagagacggggtttcactacgttggccagaatggtctcgacctcctaacctcgtgatccgcctgcctcggcctcccaaagtgctgagattacaggaatgagccaccgcacctggctaatttgtcatcttaataacaaacaactcagccaggcgcggtggctcacgcctgtaatctcagcactttgggaggccgaggtgggcagataatgaggtcaggggatccagaccatcctggctaacccggtgaaaccccgtctctactaaaaatacaaaaaatcagccaggcgtggtggcggacgcctgtagccccagctactcaggaggctgaggcaggagaatggcatgaacccaggaggcggagcttgcagtgagccgagatcgcaccactgcactctagcctgggcgacagagtgagattctgtctaaaataataaataaataaataaataaataaataacaactcataagagggactgaaactcaaacctgctggccatccatgctctgattactggatggtcgggcaacaaagggaatctgtttggggtggttagaagttgggttggagtggcctagattcgcatcctggctttaccacttgctgtttctgctcctcgtttgtaaaataaacataataataggatctactcaaggtttttagaaagatgaaatgaatcctaacaatgtgcgtagcacttcacacagtgcctggtccacatgacattctcaaattgttttaaagaacaaaatgacaactcatttttaaaaattccaaaaaaaaaaaaaaaaaaaaaaaatcccccaaacaactgtttttcccatttcttatcttcaaacatttaattcttctgatttttttttcctagaatttgttttggtttggcataggaaaagctactgtatttttacttccggctctaatttttgcggtaaaactggctaagtactatcgtcgaatggattcggaggacgtgtacgatgagtaagtatgtgggcttccacagacgtgggggtcattttctaggtggtgctgtcttcaaagagttgctaaagatgagttctgatgaggttaaagttgatttctttttttttttttttttagtagtagttgggtcacttctgttcaggtgaatttttttttcttggtactgatttttctatacttgttttaactacttttttcctcttgcttaatcctctcagcttccctaaccagcctttttatttcttgtttgcagttcctctctctcggggacctggcatttcactttatgataactgtttttacactttccattttggctctgtagtctgcccctcattctgtgtctggtgaatgtgtatgccttgtttttcacttcacttatctttcaacatgggtctttcctgagtttgcactgtcagtatccgtgttagagtaaatatttgggggatgtagttgctgagctttcataataattaaaaaaattaattttatctcccttttttgtattttatagtgttgaaactatacccatgaaaaagtaagcctttttaaaaaaactatattcctagatgggatgggggaggttgatattattatgtaatggactttaaaaattaagttattaacatcttaaaattatgtagaaacagcagatctgtacaggcaaaaataaattgcaatggtcttaaaaagagggttggcacatagagggtgctcaagaaatacttgatgaattaatgaaattttatatgttaagtttattaatctataaagtagtcctttggtctttgaagtttaaaaggagcaaaatgtcaattactcatttgtcttttttttagatttaacaaattgttacatacttttcatgaccattacattgtctttgtaatccatgatgtaaatacttcatcttagagataacatggtgtttttggtgtatttatttctgttacagtatggaaaatggtaataatggttatcataaagatcatgtatatggtattcacaatcctgttatgacaaggtaaagcaaagtctggaagtgcatgcccgccctatgataaaccgtctcatctcctatagatcagcactgaattttctgcatgctgctgatgccttcacttctctctgtatgatactttatcaatgctagtctccaatcatgctctacatagtgttccatgatgccatgagtttaaatgttcagtcgatttctaaacaatggaattattcagcaactaaaatttggtgtcatcttaaaatgctatgtatggctgtagatcattacagattaaatgcatgcataccttgcatttagaatttctctgcaccaaataacctgagtagttacagttttcaaagcttttccttccttccttctttctttccttccttccttccttccttctttccttccttccttccttttttccttccttccaataaattaactgtaaccaaccagaaaaagaacctcaattattcctccgaaatctttatgaatttacattctctagaattaaggagcttctgtggttctggatatatcatatgtcaggtaactctgagctttgcttgccagaggtcggtcctcttctgaggctctactaagaattcagacccacacgtcagccagggacctatctgcttataagaggtggagacatagtgtgcagggcagcagggcagcagtgggctgtcctcttcagtgggcgtgatccttcccacgcccatacagctggcccgccagtgctcccccgagcccgctccccacagtgatgtctcagatgcaaaagggtgagtctaacgttggtgcatgggggagatccaccttagaaggcccacgctccacatgggaggcatccagttaaaagtgcccacattcaggtaagcccacagctttggcttcacaccaggtatttatatttagagcctcccaggccagaagctgcttaccagtcaggcacatccttatcttaagcaatattacctagtaacagttgatgctccaccttggtcagatttcaagatgaacagaaccagaaagtcattaaaatttgtcctttcaagaggtaaatggcttcctgaacttaaaactttgaacattgtaaaaaaatcagaacttttcattatgattttccatttaggggaagagaagttaactgagtgcattccagctgctataatgaaatgccttaaactgggtagcttataaacatcagggtttatttctcacagttctggaggctggaatacaagatcaaggtgccatcagattcggtgtccggtgaaggaccactttctcataggtggcgttgtcttgccaaatcctcacatggtggaaagggcaagctggctccttggggtctattctctaagggcgttaatgagagactaatgagggctttgccttcatgacctaaagcgtcaccgtgattatgacgtcaatgtatgaattttaagagtgcacaaacattcagatcagagaagaaatggtgataattaaaaaaatcaagttcttggccaagcgcggtggctcatgcctgtaatcccagcacattgggaagccgaggcgggtggatcacgaggtcaggagatcaagaccatcctggctaacacggtgaaaccttgtctctactaaaaatacaaaaaattagccaggtatggtggcacacgcctgtagtccctgctacttgggaggctgaggcaggagaatcacttgaacccgggaggcagaggttgcagtgagctgagatcgtgccactgcattccagcctgggtgacagagggagactctgtctctaaataaataaataaagtcatgaaatggcaaattgacttcatctcatctgatacttccagtatgcaaggcttggtggctagagtgctgtgttgtgacagagttgaagatcgatttaagtatcaattagccaggtgtgctctggggagagtagacagtacggctgcctgcataatgtcctggccttcctttttgcggtgtagcatgcttggagggtgtcttatatatatggactgaaattttcaagatcagaggacagctgttctcaattacctatgttgccctaacatcggatcttctaggactgctttgaatttgtagaattgcttcctggtgtttgcctgttggttgtgaaggcactgaaagtactctctgttctaagaagagtcatccaggctgcctggaagatgctagagagagagatgcaagcctgggtggaggctgccacaggcaggctttctggaagcagtgctcaaatggagtttggagtgcaggatgtttatgaggagccatggttgtgaaaaaaagggggagaagtcaaggttggcctgccaaagccttggcccgactaggcaggagctctggatcaagcatggcctgtagggagttgcccacactgggctaatcattacagattagctaagtctttgcaccccctcctcccccagtcactgctcccagggctggagcagcactgccatagagctgtaggctctgctgtcagagtggtaggctctgacagctggaggctctctgctgaccatgctgcccccagctggaggcaagtcattcctaaaagggaatctagaaggcacaactccacggctccctgagaagcagatgcactggtaggcctggaagaatgtgctgcatccaataagtcctcctgcaattgaaagggaggtgtcagtgtgcaagttgacaagacagcctttgagatgccagggtcctctcagcacaccagtactgccacagacctttgtcacttaggcatgatcatcacagccaactggactctggctaatccccagcttcagatctgagtgctggaaataggcaggcgtcagctgtcttctgtcttatgccaccacgtgagacctttcccacaaaggagggtagacttccagttctagcatctagaagagaataaccgagcacctattagtgccacagatgtcctgcgtgtagcgtcaaagacttaattctctaggctcaagaaggttgtcgtagctctccttatttactagtttttactcacattgctggaacagcataagacttgataactcttggatttaacttgctgtacaagaaaaaaggcatgatgatggaaaacaggaaaatgttagaaaacagccctagtcggtgttgagaaatgccactcttctcacttgctaatgaacatttcatcttgggcttttagtgttgaaattcgaactagattaaatgccattgttctattaaatacataaatgaagctttaggatgattctgtggtcattctgcttacgacagacattggaaggacttgtctttgttaagggcctagcacttcatacacctgtgaagcctggggctagggatatactaatgttggacgtccttccttaacggtccacagcacacttgaatttttttctactttctatattctgtgttctatagtagaactccaagggtgggttgaagtgttttgtttcagtaagagaagaatcccaaagactgagtgacaccttatgtgagtttcgctgaagcatctttcatggtaacaattaaatcgagacttgttttcattcctgcacacccacctccagtgggaaccctggcaagtctcctggatgttcagcccgtctacaggcagtcagtgaagaatcatagttttgatttggtttatttgcagaaggttaggaaatgacatcccagaaactctagggacattgggacacctttcttttccacccagaccctcctagaggtcccaggggtctgagttggagtggattctagctctgcctccatgtgtcattgtggaagtgctatttaatgtccttgagactcagttttctcacttgtagaaggagggaataataagagctttgaggctgggtgtggtggctcacgcctgtaatcccagcactttgggaggccaaggcaggcaaatcacaaggtcaggagtttgagaccagcctggccaacatggtgaaacctcatctctactaaaaatacaaaaaattagctgggcgtagtggcgggcacctgtaatcccagctacttgggaggctgaggcaggagaatcgcttgaacccgggaggcggaggttgcagtgagctgagattgcaccaccgcactccatccaggacgacagagtgagactccgtctcaataaataaataaaaataagagccttgtttgcgtagggttgtgagggttcaatgaggtaacaaggcaaaacattcaccctttgaccactgtaaacccttacttaagggtttgtggttcactataatgaccataatcttttcttattaccttaacatttccaatggaaggaacccaaaggtgattcagaagataactaagtgtggctctaatctgttagctgagccagctccacttagcttccattgatgacaccaactgggctctggatattagccgacacctcatgataccttctgtacaaatgagcccgaattggtgcctccttcacacagcttgagactgtgatttcccccagtgtttatcgccaacagttttcaggtgttgcctgcatttcaggacaaagctaggtttaggaggaaaatgatcagtgcaaagagaaggtgctaggctattctagaaaagactctggctgatgcggtggctcacgcctttaatcccagcaccttgggaggctgaggacagaggatcacttgagcccaggagttcaaaaccagcctgggcaacacaacaagaccctgtgtttataaatgaaaaaaaaaattatttaatgtaagaacaaaagaaaagccttctgcaaaccaggacactagtacttaccaagtgaggagcagtttgacaactaatattgcttcatggagcgtttcccaggagaattctcttaaataagaaccatgaagtgggctgcttctgagtggcaaactcagtttcataaactagttttttttggtctctttttcttttatttctatttatttcttaaacacacacacgcacactgtaattagtccattatacagtctgtggctgtttgcttttgtttgaaggaaaagttaatagactttcatataaacctacgtaagcctggattttttttttccaacgatgcttctgtctcccacagtctgcggagcaagtggaggcattttcatttcaggaaatattgggtgacttgtttctgaatcttcttcctggcctttcatggatcactagaggtcactcggattgtgatctttcagctgctagggagttctttagagagagagagagagagagagagaaagagagagagtgtgtgtgtctgtgtgtgtgtgtctgtatgtgtgtgtgtaaacttactctttctagctctgagatgagagatcaggagagaaagttgcttcctgtgagaccacaaaggcttgagagcaaacaaacaccagtgttggtccagcagccccaggaaccgggcaggatgaccaccctgcctcatcctgcgcagtccccacatcggccctctatggctgtcgtcaccgccttgttgtctgtcttttgcagatagactcagggacgtgtaggccatgcccaggtctctacagctagccagggccaagctatgatttgagctatttctcctattctcccaaaggcaggtgggcaaagaaaagttcccaaaacgggtggaaagaccacagaaaaatttgattatttttcccagtcttcactctgaagactatcaaattggcagaaaggggaacaaataagaggcaagtgttgctagaaactctatctgaaaaggacagaaatctgattttcatatgaaattaccctttttaatgttagcaaacaatgtggagaaaaaaacaccatgggggcccaacaaaatatgtctgtgaaccaccttcttgctagttcggctgtagatggtgttcattgtgcatgctctgtagattctttgctgttgttgttgtttgagacacagattccactctgtcacacaggctggaatgcagtggtgtgatctcagctcactgcaacctccacctcctgggttcaagcgattcttgtgcttcagcctcctgagtagctgggattacaggcacacaccaccgtgctcagctaatttttctgttattagtagagatggagtttcactatattggccaggctggtcttgaactcctgacgtcaagtgatctacccaaattgctagattacaggcatgagccactgcgcttggccagattcttagatagaagtaggaggggtccgcatggctgcacctctgcagcctagaggaggaagcaggtggatcccggggaatgctacataaacagcagctgaccctcacctgtgtaggttctggctgcataggtcttgtagagggtgcttggagccccttcccttgtgtgatgtttcaggttcttcgggggccaggaccttcctttttctgtgaaggggatagtgcatcattgacaaacagattccacctcctgggccaacttctctctgttgtccggctgctcagagccctgttgtgttgtgttgattgaaatctggaggtttaatccagactgactcaaaagtactgtcatcagtgtgtgatgtctactctggagacaggagagtctcagatgtcatgtattggccttctttattttattgctttattgtgttgggcacccaggaagttatcattcagagcccaaatcctaatggcctgaagaaatgtatcctccagttatggtctcacctaaggccctttgtttctcttacatcaggagcttccaggattagttttagttacttgctatagtttcagtgtcccctccaaaactcatgttgagatttaattgccattgtgactatattaacaggcgagaccttggacaggtggttaagtcatgagagttctgccttcataaatggattaatgccattatcatgggagtgggctcctgataaaaggaggaagctcgaccccattccctgtctgtctcttgcactcatttgcccttccattatgggatgatgcgtcatgaaagccctcaccagatgccagcaccaggttcttggacttcgtggccttcagaactgtgaaaaacatatttttttctttgtaagttacccagtctctgatattctgttatagcagcagaaaacatactaagacactgcttaaattggcttgttttgctcatttcctgcttctgaggtctccagagttccctctcttccaggataggatggatatactgtctggaggagaggcaatgctaggcaggaggagaagaaccagtgatcctgttagtgtgcccagcatccctctgctcaataaggcaaatgtagatgttctttggaaatgattgccagagcctacagtccttcataatgttcagctccaggtcctggtgaggtggacagtaacaagagattgagccagcccctgcttgcacccttttccaggggccctttgagccctttcgataaagagtttgtttctttaccgaaagcataaaaggtagtgcaagaagaggcacacagaaccaagcatgtgcactttctaaagtgaaaaactttagaaggaatgtcttgcctggcagatgccctgatgtcctccgtgcatgtcctcccccaacctcatgtgtctgatgcagcactgcagtgaggcacggtggatcctgcaggttcagaggggttgtgatccacctgaagtcacagtcgggcagggtgtgccagagttgaaactgccgaccttccagatggagttcactagtctccccctggcgtatacctttttggttgtcaaggccattatcacagagtcactttagaggacactagggctactgttgatggttcacccagagagccacttagaggccgtgtgcactgcctgatcttacacccaggaatttgtagttgtaacaaggtccctgcggaacttccatttgcttctgtcttctgtcctagaagtggactttagaaagactcttgtggtagacatgtgtactgctgtgttgctgctggtctaatgtcttcaacacgtattcttagtgactcttaacacttttaaccttagattgtgaatattcacaattcttacttgtcatagtctgtttatcctctttatcttctatgctgtttttaaaagcaatacaaacagaagctagagtaaccagtgttttgtctgacacattcttcttgataaactttatgaggtgtggtttatattcgtctgtacctttagtcacatgcctgcttcttagcacagaggtgatgaaaccacccacagctttgttgggggtattatatttctttatttttttgttttttagcccatcacaacattgatagctgatgttgaaactgcttgaggtaagtttgatgctttcattgaagagaatctacaggactatagtttttagtttttagaacatcaaaacatagctattcctaatacattgccacattaagtgggaaatctagatcagcatattagacaagatattttatatgtccagtccaccctctgtacctgcaggttccacatccaccgattgattcaaccaactgtggatcaaaaatactctaaaaaaatttttttttaaataacagtacaaaaataaaaataataaaaacaatacagtataacagctacttacataacacttacattgtattaggtattttgagtgatctagagatgatatacagtatgtaggaggatgtgtgtagcggtcctggaaccaatcccacatggatccagagggacgactgcatgtatacatacgatacagtgaccctctctgtatatatatgcattgctatataaatgtgtgtgtatttatatatatatatacacacacacagtgagtatgtgggtgtatattaaaaaaataaaatttttttacttaaaaagataagattttttaagtaaaaaaataagggctggtgcggtggctcacgcctgtaatcccagcactatgggaggccgaggcgggcagatcacttgaggtcaggagctcaaaaccagcctggccaacatggtgaaaccctgtctctactaaaaaaagttagccgggtgtggtggggggcccctgtaatcctgcctgtaatctcagctactcaggaggccaaggcaggagaatcgcttcaacctgggaggcagaggttgcagtgagccgagatcacaccactgcactccagcgtgggcaacagagtgagactccatcacaaaaaaaaaaaaaaagaaaagaaaagaaagtgtttttttagagtaaaacaataagccaggcatggtggtacctgcctgtagtcccagctactggggtgggaggctgtggtagaaggattgcttgagcccaggagttcaaagatgcagtgaaccatgattatacctgtgaatagccaacacactcatgcactccaacttggaaaatgtagtgaaaccctatctctaaacaaacaaacaaacaaacaaaaatatagaaatctgtgtaccaaccacgtaccttgagtattatggacttaaaattttttctggctgggcatggtagctcatgcctgtaatcccagcactttgggaagctgaggtgggcagatcacttggggttaggagttcgagaccagcctggccaacatggagaaaccccatctctactaaaaaatataaaaattagccaggcatggtggcaggtgcctgtagtcccagctacttgggaggctgaggcttaaacccgggagacggaggttgcagtgagctgagatcgcgccattgcactccagcctgggcgacagagtgagactccctctcaaaaaaataaataaataaaataagataaataaacaaaaaccacaaagtttttttctatattgctatattttaacagattgctttgcaacaaacatattgctgtgcctcttgaacgagtttatattttccttagttcttaggctcctggtggggaccattttaggattatgcattcctggttttctctttattaattcaaaagcaatttctaaaattgtgtttaactttcatttcagcatcaggatactcaaagtggaaaggatcacagatttttggtagtttctgggtctacaaggactttccaaatccaggagcaacgccagtggcaacgtagtgactcaggcgggcaccaaggcaacggcaccattggtctctgggtagtgctttaagaatgaacacaatcacgttatagtccatggtccatcactattcaaggatgactccctcccttcctgtctatttttgttttttacttttttacactgagtttctatttagacactacaacatatggggtgtttgttcccattggatgcatttctatcaaaactctatcaaatgtgatggctagattctaacatattgccatgtgtggagtgtgctgaacacacaccagtttacaggaaagatgcattttgtgtacagtaaacggtgtatataccttttgttaccacagagttttttaaacaaatgagtattataggactttcttctaaatgagctaaataagtcaccattgacttcttggtgctgttgaaaataatccattttcactaaaagtgtgtgaaacctacagcatattcttcacgcagagattttcatctattatactttatcaaagattggccatgttccacttggaaatggcatgcaaaagcaatcatagagaaacctgcgtaactccatctgacaaattcaaaagagagagagagatcttgagagagaaatgctgttcgttcaaaagtggagttgttttaacagatgccaattacggtgtacagtttaacagagttttctgttgcattaggataaacattaattggagtgcagctaacatgagtatcatcagactagtatcaagtgttctaaaatgaaatatgagaagatcctgtcacaattcttagatctggtgtccagcatggatgaaacctttgagtttggtccctaaatttgcatgaaagcacaaggtaaatattcatttgcttcaggagtttcatgttggatctgtcattatcaaaagtgatcagcaatgaagaactggtcggacaaaatttaacgttgatgtaatgaaattccagatgtaggcattccccccaggtcttttcatgtgcagattgcagttctgattcatttgaataaaaaggaacttggaaaacatggtctgtatgttacttccttgagtagatccagcgactcacaacttagatgatgaaatttgagcatgtgttatttgtgaattccattaattattattaaagggcccattatacagaagtaattcaaggtcaaggtcaaagtgggctatattttagatcttcttcagatctgaagtcatcatttcttagggaactgtccaggtcacaatatatacaattggataccaactataggcaaactctactttgcctgatgtaatcctaaacatacaccagtaaaaatgaatttttttaaaacaacacacaagaggcttgcaatttaaacaagactatatagtgtataattcttctggaggaaattatctcttaacttcccattttttgaaaacctgatttttgtatcacccatttaaatattaattcaagttattcaagccaacttaatactcctagtgtcactcttgcaacttttatttatttatttttcagtatctcaagcctaaagttcttatctttgctaaaaaaaaaaaaaaaaaaatcagcctggccgggcgcagtggctcacgcctgtaatcccaacactttgggaggccgaggtgggtggatcatgaggtcaggagatcgggaccatcctggccaacatggtgaaaccccatctctactaaaaatacaaaaattagctgggtgtggtggtgggcacctgtagtcccagctactcgggaagctgaggcaggagaatcgcttgaacctggaaggcagaggttgcagtgggctgagatcgcaccactgcactccagcctgctgagagagcgagactctgtctcaaaaaagaaaaaaaaatcagcctttcctcaaccccattgcacactcagcctggtagtacaagatgttttcttttttccctctctctttggaaataatttccttgaaagaactctcgacacatctctgtccactttctcatttccattggttcttcaactcctgtgacctgccttctgtccccaccactcctgaagggctattgtcggggtcaccgaggacttgacttttgtgaacacatcccctttacaaagatctcccttcaggctcccaaaagagccctctccagtgtcctcttggggcatggctgtctattcctcagtctccttccaacactcatctctccagttcttaaatttaggtggcattcgcaggttctctcctggtgtgcccctcactctctccttgggtaatttcattcactcatcacctcacctgctccctttggttctctcatcacatagagctccagccaaccctgtgttcatgacatgtctccacccagatgcaccttaaaccagaggttggcaacctttctctgaaaagggtcagacagtgaatatattttgctttctgggccatgtggcctatgtctcctcaactctgttgcagcacaaaagtagccataggcaatacgtaagtgaatagatattgccgtgttcttcaaaaattacaaaagcaggccacatgggccacagtttgtggcccctgccttaaaccactgtcctgacctgcacatcgccacaggttctccccaccatgttccctttcttgaatttctccaccttcaccctatcactggagctggaggaactcttaagtctgctttccccctcccttccatctacctttggtcacaaagccttcctctccatccccactgatgagatcctcttacagttttcataatgtctcttggctagcgaggcattcccctctttcttttaggcttccagccccctgagttttggctagacataggctacctggtggggactatatttcccagcctcccttgcagctagatgtggctgagtgactaggcttaggccagcagtaagtaagtgaagtgtctactaagacaaaactgcagatcctggacttgttccactggaaatggcagcaatcaaacaaaccttggaaggcatgtgttaaatttgaagaacttccaggcatgaacc

SEQ ID NO: 10 - DNA, 20 bp, Artificial
  Synthetic DNA primer for Sox2 in forward direction.
agtacaactccatgaccagc

SEQ ID NO: 11 - DNA, 19 bp, Artificial
  Synthetic DNA primer for Sox2 in reverse direction.
tcacatgtgtgagaggggc

SEQ ID NO: 12 - DNA, 19 bp, Artificial
  Synthetic DNA primer for Oct4 in forward direction.
taagcttccaaggccctcc

SEQ ID NO: 13 - DNA, 19 bp, Artificial
  Synthetic DNA primer for Oct4 in reverse direction.
ctcctccgggttttgctcc

SEQ ID NO: 14 - DNA, 20 bp, Artificial
  Synthetic DNA primer for Klf4 in forward direction.
aattacccatccttcctgcc

SEQ ID NO: 15 - DNA, 23 bp, Artificial
  Synthetic DNA primer for Klf4 in reverse direction.
ttaaaaatgcctcttcatgtgta

SEQ ID NO: 16 - DNA, 20 bp, Artificial
  Synthetic DNA primer for c-Myc in forward direction.
tccactcggaaggactatcc

SEQ ID NO: 17 - DNA, 21 bp, Artificial
  Synthetic DNA primer for Sox2 reverse direction.
ttacgcacaagagttccgtag

(19) United States
(12) Patent Application Publication
(10) Pub. No.: US 2011/0044961 A1
(43) Pub. Date: Feb. 24, 2011

(54) Generation of Induced Pluripotent Stem Cells from Cord Blood

(75) Inventors: Alessandra Giorgetti, Barcelona (ES); Juan Carlos Izpisua Belmonte, La Jolla, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Salk Institute for Biological Studies, La Jolla, CA (US); Center of Regenerative Medicine, Barcelona (ES)

(21) Appl. No.: 12/819,059

(22) Filed: Jun. 18, 2010

Related U.S. Application Data
(60) Provisional application No. 61/218,611, filed on Jun. 19, 2009.

(51) Int. Cl. A61K 35/12; C12N 5/071; C12N 5/0789; A61P 43/00

(52) U.S. Cl. 424/93.21; 435/377; 435/325; 435/372

(57) ABSTRACT

Methods and compositions for the generation and use of genetically corrected induced pluripotent stem cells are provided.

### CROSS-REFERENCES TO RELATED APPLICATIONS

This application claims the benefit of U.S. Provisional Application No. 61/218,611, filed Jun. 19, 2009, the content of which is incorporated herein by reference in its entirety and for all purposes.

### BACKGROUND OF THE INVENTION

Ectopic expression of pluripotency factors and oncogenes using integrative viral methods is sufficient to induce pluripotency in both mouse and human fibroblasts.³⁻⁹ However, this process is slow, inefficient and the permanent integration of the vectors into the genome limits the use of iPS cells for therapeutic applications.¹ Further studies have shown that the age, origin and cell type used has a deep impact on the reprogramming efficiency, eventually requiring the expression of less factors and/or reducing the timing of the whole process. Recently, it was shown that retroviral transduction of human keratinocytes resulted in reprogramming to pluripotency which was 100-fold more efficient and twice as fast when compared to fibroblasts. It was hypothesized that these differences could result from the endogenous expression of KLF4 and c-MYC in the starting keratinocyte population and/or the presence of a pool of undifferentiated progenitor cells presenting an epigenetic status more amenable to reprogramming.¹⁰ This latter hypothesis has been further supported by other studies in mouse.^{11,12} However, stem cells are usually rare and difficult to access and isolate in large amounts (e.g., neural stem cells^{13,14}).

Induced pluripotent stem (iPS) cells have generated interest for regenerative medicine, as they allow generating patient-specific progenitors in vitro with potential value for cell therapy.¹ However, in many instances an off-the-shelf approach would be desirable, such as for cell therapy of acute conditions or when the patient's somatic cells are altered as a consequence of a chronic disease or ageing. Cord blood (CB) stem cells appear ideally suited for this purpose, as they are newborn, immunologically immature cells with minimal genetic and epigenetic alterations, and several hundred thousand immunotyped CB units are readily available through a worldwide network of CB banks.² CB cells, considered an alternative to bone marrow (BM) as a source of stem cells for haematopoietic transplantation, can be collected in sufficient amounts without any risk to the donor.¹⁵ In addition to being easily accessible, CB cells combine the characteristics of being young cells with minimal somatic mutations with the advantage given by the immunological immaturity of newborn cells.¹⁵ These properties allow for less stringent criteria for HLA-donor-recipient selection, representing a decisive benefit for transplantation. Moreover, a worldwide comprehensive network of cord blood banks guarantees a fast and effective search for compatible donors for CB stem cells.² Finally, CB CD133+ stem cells have been shown to express OCT4, SOX2, NANOG, REX1 and other pluripotency-associated markers¹⁶⁻¹⁸ and therefore may be, in principle, more amenable to reprogramming. Here it is described for the first time, embodiments including the fast and efficient reprogramming of CB stem cells to pluripotency by retroviral transduction of four (OSKM), three (OSK) and as few as two (OS) transcription factors without the need of two potent oncogenes (c-MYC and KLF4) or additional chemical compounds.

Using certain methods and compositions described herein, CB stem cells can be reprogrammed to pluripotency by retroviral transduction with OCT4, SOX2, KLF4, and c-MYC, in a process that is extremely efficient and fast. The resulting CB-derived iPS (CBiPS) cells are phenotypically and molecularly indistinguishable from human embryonic stem (hES) cells. Furthermore, the generation of cord blood iPS can be efficiently achieved without the use of the c-MYC and KLF4 oncogenes and just by overexpression of OCT4 and SOX2. The methods and compositions described herein overcome the problems in the art and may set the basis for the creation of a comprehensive bank of HLA-matched CBiPS cells for off-the-shelf applications.

### BRIEF SUMMARY OF THE INVENTION

Provided herein are, inter alia, highly efficient methods and compositions for making and using induced pluripotent stem cells from cord blood.

In one aspect, a method for preparing an induced pluripotent stem cell is provided. The method includes transfecting a cord blood stem cell with a nucleic acid encoding an OCT4 protein and a nucleic acid encoding a SOX2 protein to form a transfected cord blood stem cell. The transfected cord blood stem cell is allowed to divide thereby forming the induced pluripotent stem cell.

In another aspect, a method for preparing an induced pluripotent stem cell is provided. The method includes transfecting a cord blood stem cell with a nucleic acid encoding an OCT4 protein to form a transfected cord blood stem cell. The transfected cord blood stem cell is allowed to divide thereby forming the induced pluripotent stem cell.

In another aspect, an induced pluripotent stem cell prepared in accordance with the methods herein is provided.

In one aspect, a cord blood stem cell including a nucleic acid encoding an OCT4 protein and a nucleic acid encoding a SOX2 protein is provided.

In another aspect, a cord blood stem cell including a nucleic acid encoding an OCT4 protein is provided.

In one aspect, a method for producing a human somatic cell is provided. The method includes contacting an induced pluripotent stem cell with cellular growth factors. The induced pluripotent stem cell is allowed to divide, thereby forming the human somatic cell. In some embodiments, the induced pluripotent stem cell is prepared by a process including the steps of transfecting a cord blood stem cell with a nucleic acid encoding an OCT4 protein and a nucleic acid encoding a SOX2 protein to form a transfected cord blood stem cell. The transfected cord blood stem cell is allowed to divide thereby forming the induced pluripotent stem cell. In another embodiment, the induced pluripotent stem cell is prepared by a process including the steps of transfecting a cord blood stem cell with a nucleic acid encoding an OCT4 protein to form a transfected cord blood stem cell. The transfected cord blood stem cell is allowed to divide thereby forming the induced pluripotent stem cell.

In another aspect, a method of treating a mammal in need of tissue repair is provided. The method includes administering an induced pluripotent stem to the mammal and allowing the induced pluripotent stem cell to divide and differentiate into somatic cells in the mammal, thereby providing tissue repair in said mammal. In some embodiments, the induced pluripotent stem cell is prepared by a process including the steps of transfecting a cord blood stem cell with a nucleic acid encoding an OCT4 protein and a nucleic acid encoding a SOX2 protein to form a transfected cord blood stem cell. The transfected cord blood stem cell is allowed to divide thereby forming the induced pluripotent stem cell. In another embodiment, the induced pluripotent stem cell is prepared by a process including the steps of transfecting a cord blood stem cell with a nucleic acid encoding an OCT4 protein to form a transfected cord blood stem cell. The transfected cord blood stem cell is allowed to divide thereby forming the induced pluripotent stem cell.

### BRIEF DESCRIPTION OF THE DRAWINGS

FIGS. 1A-1E. Generation of CBiPS cell lines using only OCT4 and SOX2 factors. FIG. 1A: Timeline of cord blood stem cells reprogramming. Three days post infection, CB CD 133+ cells are transferred on feeders. Small adherent colonies are observed around day 9. Typical hES-like colonies are clearly visible after 12 days. FIG. 1B: Genomic DNA PCR confirming the insertion of 4, 3, and only 2 transgenes. FIG. 1C: Representative phase contrast images and Alkaline Phosphatase (AP) staining of CBiPS2F-1, 3F-10 and 4F-3 cell lines. FIG. 1D: Representative telomerase activity in CBiPS2F, 3F and 4F cell lines (HI: Heat Inactivation, HFF: Human Foreskin Fibroblast, —C: lysis buffer as negative control, +C: positive control and QC: Quantitative Control). FIG. 1E: Immunofluorescence analysis of CBiPS2F-1 cell line for pluripotency markers. The colonies express the embryonic markers SSEA-4, SSEA-3, TRA-1-60, TRA-1-81 and the transcription factors OCT4, SOX2 and NANOG. Underlying fibroblasts provide a negative control. Scale bars, 250 μm

FIGS. 2A-2G. Characterization of CBiPS cell lines. FIG. 2A: Histogram depicting quantitative RT-PCR analysis for pluripotency markers OCT4, SOX2, NANOG, REX1, CRIPTO, KLF4 and c-MYC. ES[2] and Keratinocyte-iPS (KiPS) cell lines were analysed together with the different CBiPS cell lines derived from fresh and frozen samples. Error bars indicate the s.d. (standard deviation) generated from triplicates. Histogram legend (left to right):CBiPS4F-3, CBiPS4F-5, CBiPS3F-10, CBiPS3F-12, CBiPS2F-1, CBiPS2F-2, CBiPSF-1, CBiPSF-5, ES2 and KiPS. FIG. 2B: Histogram depicting quantitative RT-PCR showing the repression of the OCT4, SOX2, KLF4 and c-MYC transgenes in the CBiPS cell lines. Histogram legend (left to right): CBiPS4F-3, CBiPS3F-10 and CBiPS2F-1. FIG. 2C: In vitro differentiation of CBiPS 2F-1 into the three primary germ cell layers (Ectoderm-Tuj 1, Endoderm-AFP and FOXA2, and Mesoderm-ASA and GATA4). FIG. 2D: Immunofluorescence analysis of teratoma sections 60 days after intra-testicular injection of CBiPS2F-1 showing Tuj1/GFAP positive ectoderm, AFP/FoxA2 positive endoderm and ASM/ASA positive mesoderm. Scale bar 75-250 μm. FIG. 2E: Specific in vitro differentiation of CBiPS2F-1, and FIG. 2F: CBiPS3F-12 into dopaminergic neurons (Tuj 1/TH tyrosine hydroxilase), which are immunophenotypically mature. FIG. 2G: Histograms depicting chromatin immuno-precipitation assays comparing the levels of histone H3 methylation at K4 (H3K4me2), K27 (H3K27me3) and K9 (H3K9me3) in the promoters of OCT4, NANOG, HOXB4 and HOXB5 in human fibroblasts and CD133+ cells. Histogram legend: Fibroblast (arrayed dots), CD133+ (black).

FIGS. 3A-3C. Flow Cytometry analysis of human CD133+ cells. FIG. 3A: Depicts representative dot-plot for CD133 cells purity after immuno-selection. FIG. 3B: Depicts quantification of total GFP+ cells and double positive GFP/CD133 cells three days post infection. FIG. 3C: Histogram depicts flow cytometry analysis of untransduced CD133+ stem cells cultured for 3 weeks in hES conditions. Cells were analysed for the haematopoietic markers CD45, CD34, CD38 and CD133, and for embryonic stem cell markers, including SSEA3, SSEA4 and TRA-1-60.

FIGS. 4A-4B. Scheme of pMXs-OSKMG and pMXs-OSKG polycitronic retrovirus. FIG. 4A: pMXs-OSKG polycitronic retrovirus. FIG. 4B: pMXs-OSKMG polycitronic retrovirus.

FIGS. 5A-5C. Immunofluorescence analysis for pluripotency markers. FIG. 5A: CBiPS3F-10. FIG. 5B: CBiPS4F-3 cell lines express other typical pluripotency markers including SSEA-4, SSEA-3, TRA-1-60, TRA-1-81 and the transcription factors OCT4, SOX2 and NANOG. FIG. 5C: CBiPS frozen (CBiPSFr)-1 cell lines, generated using CD133+ cells purified from frozen/thawed CB units, after transduction with OSK retroviruses, express other typical pluripotency markers including SSEA-4, SSEA-3, TRA-1-60, TRA-1-81 and the transcription factors OCT4, SOX2 and NANOG. Underlying fibroblasts provide a negative control. Scale bars, 250 μm

FIG. 6. Flow Cytometry analysis of CBiPS2F. Histogram depicts low cytometry analysis confirming that CBiPS2F-1 cells have lost haematopoietic markers such as CD45 and CD34 and acquired typical pluripotency markers including TRA-1-181 and SSEA-4.

FIGS. 7A-7B. Global gene expression analysis. FIG. 7A: Average global gene expression patterns were compared between CBiPS (2 lines, 2 replicates each) and ES2 (2 replicates), showing a very high level of correlation. Some pluripotency genes are identified in the plot. FIG. 7B: Correlation coefficients of genome-wide transcriptional profiles for all pairwise comparisons of different pluripotent lines and the respective starting populations.

FIGS. 8A-8B. Retroviral transgenes silencing. Immunofluorescence staining against OCT4 in combination with a specific FLAG-antibody which only detects transgene expression from any of the FLAG-tagged retroviral transcription factors. FIG. 8A: Expression of the endogenous OCT4 and silencing of the transgenes in CBiPS2F-1. FIG. 8B: Primary human fibroblasts infected with OCT4 and SOX2 as positive control. Scale bars, 250 μm

FIG. 9. Methylation promoter analysis by bisulfate genomic sequence. OCT4 promoter methylation analysis confirming consistent demethylation of the promoter in all CBiPS cell lines.

FIG. 10. Southern blot. Southern blot to assess the number of retroviral integrations in the CBiPS2F-1 line and subclones. 1: Genomic DNA digested with PstI hybridised with a KLF4-specific probe. Endogenous bands: 5.9 kb and 0.9 kb (black arrowheads). As expected, no additional bands are detected with this probe. 2: Genomic DNA digested with PstI hybridised with a SOX2-specific probe. Endogenous band: 0.9 kb (black arrowhead). The same additional band is present in CBiPS2F-1, CBiPS2F-1a and CBiPS2F-1b corresponding to a unique transgene insertion (red asterisks). 3: Genomic DNA digested with HindIII hybridised with an OCT4-specific probe. Endogenous specific band: 4.5 kb (black arrowhead). Endogenous unspecific bands (grey arrowheads). The same additional band is present in CBiPS2F-1, CBiPS2F-1a and CBiPS2F-1b corresponding to a unique transgene insertion (red asterisks). 4: Genomic DNA digested with HindIII hybridised with a c-MYC-specific probe. Endogenous band: 11 kb (black arrowhead). As expected, no additional bands are detected with this probe.

FIGS. 11A-11C. Karyotyping for CBiPS 2F, 3F and 4F cell lines. High-resolution, G-banded karyotype indicating a normal, diploid, male and female chromosomal content in (FIG. 11A) CBiPS2F-1, (FIG. 11B) CBiPS3F-10 and (FIG. 11C) CBiPS4F-3 cells analysed after passage 10.

FIGS. 12A12-F. In Vitro and In Vivo Pluripotency of CBiPS cell lines. FIG. 12A: Embryoid Bodies derived from CBiPS2F-1 cell line. FIG. 12B: CBiPS3F-10 and (FIG. 12C) CBiPS4F-3 can differentiate in vitro in the three germ layers including neural (Tuj1/GFAP), endodermal (AFP/FoxA2) and mesodermal (ASM) cells. FIG. 12D: In vitro differentiation in the three germ layer, including neural (Tuj 1), endodermal (AFP/FoxA2) and mesodermal (ASM) cells of CBiPSFr-1 cell line. FIG. 12E: CBiPS3F-10 and (FIG. 12F) CBiPS4F-3 in vivo differentiation. The resulting teratoma contained tissues representing all three germ layers: ectodermal (Tuj1/GFAP), endodermal (AFP/FoxA2) and mesodermal (ASM/ASA).

FIGS. 13A-13D. Gene expression analysis of CB CD133+ cells. FIG. 13A: Dendrogram representing hierarchical clustering of genome-wide transcriptional profiles of CD133+, keratinocytes, fibroblasts, ES cells, KiPS and CBiPS showing that CD133+ cells are not closer to pluripotent cells than fibroblasts and keratinocytes. FIG. 13B: Histogram depicting comparative gene expression analysis of pluripotency markers and KLF4 in CD133+ cells, fibroblasts and keratinocytes by Quantitative RT-PCR. Histogram legend (left to right): CD133+ (black), fibroblast (open) and keratinocyte (gray). FIG. 13C: Validation of SALL2, ZNF589, DPPA4, DNMT3A and DNMT3B genes up-regulation in CD133+ cell by Quantitative RT-PCR. Histogram legend: as in FIG. 13B. FIG. 13D: Quantitative RT-PCR analysis of c-MYC expression in CD133+ cells, fibroblasts and keratinocytes. Error bars indicate the s.d. generated from triplicates. Histogram legend: as in FIG. 13B.

### DETAILED DESCRIPTION OF THE INVENTION

### I. Definitions

The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., the NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target sequence, typically in a complex mixture of nucleic acids, but to not other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

A variety of methods of specific DNA and RNA measurement that use nucleic acid hybridization techniques are known to those of skill in the art (see, Sambrook, supra). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “transfection” or “transfecting” is defined as a process of introducing nucleic acid molecules to a cell by non-viral or viral-based methods. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods of transfection any useful viral vector may be used in the methods described herein. Examples for viral vectors include, but are not limited to retroviral, adenoviral, lentiviral and adeno-associated viral vectors.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The term “plasmid” refers to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

A “stem cell” is a cell characterized by the ability of self-renewal through mitotic cell division and the potential to differentiate into a tissue or an organ. Among mammalian stem cells, embryonic and adult stem cells can be distinguished. Embryonic stem cells reside in the blastocyst and give rise to embryonic tissues, whereas adult stem cells reside in adult tissues for the purpose of tissue regeneration and repair.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

The term “reprogramming” refers to the process of dedifferentiating a non-pluripotent cell into a cell exhibiting pluripotent stem cell characteristics.

The term “treating” means ameliorating, suppressing, eradicating, and/or delaying the onset of the disease being treated.

### II. Methods of Preparing Induced Pluripotent Stem Cells from Cord Blood

In one aspect, a method for preparing an induced pluripotent stem cell is provided. The method includes transfecting a cord blood stem cell with a nucleic acid encoding an OCT4 protein and a nucleic acid encoding a SOX2 protein to form a transfected cord blood stem cell. The transfected cord blood stem cell is allowed to divide thereby forming the induced pluripotent stem cell.

An “induced pluripotent stem cell” refers to a pluripotent stem cell artificially derived from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells. An adult stem cell is an undifferentiated cell found throughout the body after embryonic development. Adult stem cells multiply by cell division to replenish dying cells and regenerate damaged tissue. Adult stem cells have the ability to divide and create another cell like itself and also divide and create a cell more differentiated than itself. Even though adult stem cells are associated with the expression of pluripotency markers such as Rex1, Nanog, Oct4 or Sox2, they do not have the ability of pluripotent stem cells to differentiate into the cell types of all three germ layers. Adult stem cells have a limited potency to self renew and generate progeny of distinct cell types. Without limitation, an adult stem cell can be a hematopoietic stem cell, a cord blood stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

A “cord blood stem cell” refers to an adult stem cell that resides in cord blood and is characterized by a lesser potency to self renew and differentiate than a pluripotent stem cell.

The term “transfection” or “transfecting” is defined as a process of introducing nucleic acid molecules to a cell by non-viral or viral-based methods. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion the gene is positioned between two transposon linker sequences that allow insertion into the host genome as well as subsequent excision.

An “OCT4 protein” as referred to herein includes any of the naturally-occurring forms of the Octomer 4 transcription factor, or variants thereof that maintain Oct4 transcription factor activity (e.g. within at least 50%, 80%, 90%, 95%, 96%, 97%, 98%, 99% or 100% activity compared to Oct4). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Oct4 polypeptide (e.g. SEQ ID NO:1, SEQ ID NO:2 or SEQ ID NO:3). In other embodiments, the Oct4 protein is the protein as identified by the NCBI reference gi:42560248 corresponding to isoform 1 (SEQ ID NO:1), gi:116235491 and gi:291167755 corresponding to isoform 2 (SEQ ID NO:2 and SEQ ID NO:3).

A “Sox2 protein” as referred to herein includes any of the naturally-occurring forms of the Sox2 transcription factor, or variants thereof that maintain Sox2 transcription factor activity (e.g. within at least 50%, 80%, 90%, 95%, 96%, 97%, 98%, 99% or 100% activity compared to Sox2). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Sox2 polypeptide (e.g. SEQ ID NO:4). In other embodiments, the Sox2 protein is the protein as identified by the NCBI reference gi:28195386 (SEQ ID NO:4).

A “KLF4 protein” as referred to herein includes any of the naturally-occurring forms of the KLF4 transcription factor, or variants thereof that maintain KLF4 transcription factor activity (e.g. within at least 50%, 80%, 90%, 95%, 96%, 97%, 98%, 99% or 100% activity compared to KLF4). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KLF4 polypeptide (e.g. SEQ ID NO:5). In other embodiments, the KLF4 protein is the protein as identified by the NCBI reference gi:194248077 (SEQ ID NO:5).

A “cMYC protein” as referred to herein includes any of the naturally-occurring forms of the cMyc transcription factor, or variants thereof that maintain cMyc transcription factor activity (e.g. within at least 50%, 80%, 90%, 95%, 96%, 97%, 98%, 99% or 100% activity compared to cMyc). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cMyc polypeptide (e.g. SEQ ID NO:6). In other embodiments, the cMyc protein is the protein as identified by the NCBI reference gi:71774083 (SEQ ID NO:6).

A “NANOG protein” as referred to herein includes any of the naturally-occurring forms of the Nanog transcription factor, or variants thereof that maintain Nanog transcription factor activity (e.g. within at least 50%, 80%, 90%, 95%, 96%, 97%, 98%, 99% or 100% activity compared to Nanog). In some embodiments, variants have at least 90%, 95%, 96%, 97%, 98%, 99% or 100% amino acid sequence identity across their whole sequence or a portion of the sequence (e.g. a 50, 100, 150 or 200 continuous amino acid portion) compared to the naturally occurring Nanog polypeptide (e.g. SEQ ID NO:7). In other embodiments, the Nanog protein is the protein as identified by the NCBI reference gi:153945816 (SEQ ID NO:7).

A “LIN28 protein” as referred to herein includes any of the naturally-occurring forms of the Lin28 transcription factor, or variants thereof that maintain Lin28 transcription factor activity (e.g. within at least 50%, 80%, 90%, 95%, 96%, 97%, 98%, 99% or 100% activity compared to Lin28). In some embodiments, variants have at least 90%, 95%, 96%, 97%, 98%, 99% or 100% amino acid sequence identity across their whole sequence or a portion of the sequence (e.g. a 50, 100, 150 or 200 continuous amino acid portion) compared to the naturally occurring Lin28 polypeptide (e.g. SEQ ID NO:8). In other embodiments, the Lin28 protein is the protein as identified by the NCBI reference gi:13375938 (SEQ ID NO:8).

Allowing the transfected cord blood stem cell to divide and thereby forming the induced pluripotent stem cell may include expansion of the cord blood stem cell after transfection, optional selection for transfected cells and identification of pluripotent stem cells. Expansion as used herein includes the production of progeny cells by a transfected cord blood stem cell in containers and under conditions well know in the art. Expansion may occur in the presence of suitable media and cellular growth factors. Cellular growth factors are agents which cause cells to migrate, differentiate, transform or mature and divide. They are polypeptides which can usually be isolated from various normal and malignant mammalian cell types. Some growth factors can also be produced by genetically engineered microorganisms, such as bacteria (E. coli) and yeasts. Cellular growth factors may be supplemented to the media and/or may be provided through co-culture with irradiated embryonic fibroblasts that secrete such cellular growth factors. Examples of cellular growth factors include, but are not limited to FGF, bFGF2, and EGF.

Where appropriate the expanding transfected cord blood stem cell may be subjected to a process of selection. A process of selection may include a selection marker introduced into a cord blood stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected cord blood stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin, which no longer inhibits expansion and causes cell death of a transfected cord blood stem cell. Upon exposure to a toxin a cell lacking a selection marker may be eliminated and thereby precluded from expansion.

Identification of the induced pluripotent stem cell may include, but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

As afore mentioned the cord blood stem cell provided in the methods herein may be transfected with a nucleic acid encoding a OCT4 protein and a nucleic acid encoding a SOX2 protein. In some embodiments, the cord blood stem cell is not transfected with an additional nucleic acid encoding a cMYC protein, a LIN28 protein, a NANOG protein or a KLF4 protein.

In some embodiments, the nucleic acid encoding an OCT4 protein forms part of a plasmid and the nucleic acid encoding a SOX2 protein forms part of a plasmid. In another embodiment, the nucleic acid encoding an OCT4 protein and the nucleic acid encoding a SOX2 protein form part of the same plasmid. In one embodiment, the nucleic acid encoding an OCT4 protein forms part of a first plasmid and the nucleic acid encoding a SOX2 protein forms part of a second plasmid.

In another aspect, a method for preparing an induced pluripotent stem cell is provided. The method includes transfecting a cord blood stem cell with a nucleic acid encoding an OCT4 protein to form a transfected cord blood stem cell. The transfected cord blood stem cell is allowed to divide thereby forming the induced pluripotent stem cell.

In one embodiment, the cord blood stem cell is not transfected with an additional nucleic acid encoding a cMYC protein, a LIN28 protein, a NANOG protein or a KLF4 protein.

In some embodiments, the cord blood cell used in the methods provided herein expresses a CD133 antigen. A “CD133 antigen” refers to a five transmembrane domain glycoprotein, which is 120 kilo Dalton in size. A CD133 antigen may be expressed by adult stem cells and progenitor cells. The CD133 antigen is also known was PROML1, AC133, hematopoietic stem cell antigen, hProminin, prominin-like 1, prominin, RP41, MCDR2, STGD4, CORD12 or MSTP061. In some embodiments, the CD133 antigen is the protein encoded by the gene identified by the NCBI reference gi:225690512 (SEQ ID NO:9).

In some embodiments, the cord blood stem cell used in the methods provided herein is derived from fresh cord blood. “Fresh cord blood” is blood derived from the umbilical cord of a neonate, which is returned to the neonatal circulation if the umbilical cord is not prematurely clamped. Fresh cord blood as referred to herein is not cryopreserved after isolation from the umbilical cord. The term “cryoconservation” refers to the process of freezing biological material such as cord blood using liquid nitrogen thereby conserving the biological material for long time periods. In other embodiments, the cord blood stem cell used in the methods provided herein is derived from frozen cord blood. Frozen cord blood is blood derived from the umbilical cord of a neonate that has been cryo-conserved prior to being processed according to the methods provided herein.

### III. An Induced Pluripotent Stem Cell

In another aspect, an induced pluripotent stem cell prepared in accordance with the methods herein is provided. The methods described above in the section entitled “Methods of Preparing Induced Pluripotent Stem Cells from Cord Blood” are equally applicable to an induced pluripotent stem cell as provided herein.

### IV. Cord Blood Stem Cells

In one aspect, a cord blood stem cell including a nucleic acid encoding an OCT4 protein (e.g. an exogenous nucleic acid encoding an OCT4 protein or a recombinant nucleic acid encoding an OCT4 protein) and a nucleic acid encoding a SOX2 protein (e.g. an exogenous nucleic acid encoding an SOX2 protein or a recombinant nucleic acid encoding an SOX2 protein) is provided. The term “exogenous” in reference it a nucleic acid encoding a protein as used herein means not naturally occurring in the cell in which it is found (e.g. a cord blood cell). In some embodiments, the nucleic acid encoding an OCT4 protein forms part of a plasmid and the nucleic acid encoding a SOX2 protein forms part of a plasmid. In another embodiment, the nucleic acid encoding an OCT4 protein and the nucleic acid encoding a SOX2 protein form part of the same plasmid. In one embodiment, the nucleic acid encoding an OCT4 protein forms part of a first plasmid and the nucleic acid encoding a SOX2 protein forms part of a second plasmid. In some embodiments, the cord blood stem cell does not include nucleic acids encoding other transcription factors known to be useful in iPS cell formation, such as a nucleic acid encoding a cMYC protein (e.g. an exogenous nucleic acid encoding a cMYC protein or a recombinant nucleic acid encoding a cMYC protein), a nucleic acid encoding a LIN28 protein (e.g. an exogenous nucleic acid encoding a LIN28 protein or a recombinant nucleic acid encoding a LIN28 protein), a nucleic acid encoding a NANOG protein (e.g. an exogenous nucleic acid encoding a NANOG protein or a recombinant nucleic acid encoding a NANOG protein) and/or a nucleic acid encoding a KLF4 protein (e.g. an exogenous nucleic acid encoding a KLF4 protein or a recombinant nucleic acid encoding a KLF4 protein).

In other embodiments, the cord blood stem cell consists essentially of a nucleic acid encoding an OCT4 protein (e.g. an exogenous nucleic acid encoding an OCT4 protein or a recombinant nucleic acid encoding an OCT4 protein) and a nucleic acid encoding a SOX2 protein (e.g. an exogenous nucleic acid encoding an SOX2 protein or a recombinant nucleic acid encoding an SOX2 protein). Where a cord blood stem cell “consists essentially of” a nucleic acid encoding an OCT4 protein and a nucleic acid encoding a SOX2 protein, the cord blood stem cell does not include nucleic acids encoding other transcription factors known to be useful in iPS cell formation, such as a nucleic acid encoding a cMYC protein (e.g. an exogenous nucleic acid encoding a cMYC protein or a recombinant nucleic acid encoding a cMYC protein), a nucleic acid encoding a LIN28 protein (e.g. an exogenous nucleic acid encoding a LIN28 protein or a recombinant nucleic acid encoding a LIN28 protein), a nucleic acid encoding a NANOG protein (e.g. an exogenous nucleic acid encoding a NANOG protein or a recombinant nucleic acid encoding a NANOG protein) and/or a nucleic acid encoding a KLF4 protein (e.g. an exogenous nucleic acid encoding a KLF4 protein or a recombinant nucleic acid encoding a KLF4 protein). In some embodiments, the cord blood stem cell does not include nucleic acids encoding other transcription factors (e.g. other exogenous nucleic acids encoding a transcription factor or other recombinant nucleic acids encoding a transcription factor). In other embodiments, the cord blood stem cell does not include nucleic acids encoding other protein expressing genes (e.g. other exogenous nucleic acids encoding a protein or other recombinant nucleic acids encoding a protein).

In another aspect, a cord blood stem cell including a nucleic acid encoding an OCT4 protein (e.g. an exogenous nucleic acid encoding an OCT4 protein or a recombinant nucleic acid encoding an OCT4 protein) is provided. In other embodiments, the cord blood stem cell consists essentially of a nucleic acid encoding an OCT4 protein (e.g. an exogenous nucleic acid encoding an OCT4 protein or a recombinant nucleic acid encoding an OCT4 protein). Where a cord blood stem cell “consists essentially of” a nucleic acid encoding an OCT4 protein, the cord blood stem cell does not include nucleic acids encoding other transcription factors known to be useful in iPS cell formation, such as a nucleic acid encoding a cMYC protein (e.g. an exogenous nucleic acid encoding a cMYC protein or a recombinant nucleic acid encoding a cMYC protein), a nucleic acid encoding a LIN28 protein (e.g. an exogenous nucleic acid encoding a LIN28 protein or a recombinant nucleic acid encoding a LIN28 protein), a nucleic acid encoding a NANOG protein (e.g. an exogenous nucleic acid encoding a NANOG protein or a recombinant nucleic acid encoding a NANOG protein) and/or a nucleic acid encoding a KLF4 protein (e.g. an exogenous nucleic acid encoding a KLF4 protein or a recombinant nucleic acid encoding a KLF4 protein). In some embodiments, the cord blood stem cell does not include nucleic acids encoding other transcription factors (e.g. other exogenous nucleic acids encoding a transcription factor or other recombinant nucleic acids encoding a transcription factor). In other embodiments, the cord blood stem cell does not include nucleic acids encoding other protein expressing genes (e.g. other exogenous nucleic acids encoding a protein or other recombinant nucleic acids encoding a protein).

In some embodiments, the cord blood stem cell expresses a CD133 antigen. In other embodiments, the cord blood stem cell is derived from fresh cord blood. In some embodiments, the cord blood stem cell is derived from frozen cord blood.

### V. Methods for Producing Human Somatic Cells from Footprint-Free Human Induced Pluripotent Stem Cells

In one aspect, a method for producing a human somatic cell is provided. The method includes contacting an induced pluripotent stem cell with cellular growth factors. The induced pluripotent stem cell is allowed to divide, thereby forming the human somatic cell. The induced pluripotent stem cell is allowed to divide in the presence of appropriate media and cellular growth factors. Examples for cellular growth factors include, but are not limited to, SCF, GMCSF, FGF, TNF, IFN, EGF, IGF and members of the interleukin family. The induced pluripotent stem cell is prepared in accordance with the methods provided by the present invention. In some embodiments, the induced pluripotent stem cell is prepared by a process including the steps of transfecting a cord blood stem cell with a nucleic acid encoding an OCT4 protein and a nucleic acid encoding a SOX2 protein to form a transfected cord blood stem cell. The transfected cord blood stem cell is allowed to divide thereby forming the induced pluripotent stem cell. In another embodiment, the induced pluripotent stem cell is prepared by a process including the steps of transfecting a cord blood stem cell with a nucleic acid encoding an OCT4 protein to form a transfected cord blood stem cell. The transfected cord blood stem cell is allowed to divide thereby forming the induced pluripotent stem cell.

In another aspect, a method of treating a mammal in need of tissue repair is provided. The method includes administering an induced pluripotent stem to the mammal and allowing the induced pluripotent stem cell to divide and differentiate into somatic cells in the mammal, thereby providing tissue repair in said mammal. In some embodiments, the induced pluripotent stem cell is prepared by a process including the steps of transfecting a cord blood stem cell with a nucleic acid encoding an OCT4 protein and a nucleic acid encoding a SOX2 protein to form a transfected cord blood stem cell. The transfected cord blood stem cell is allowed to divide thereby forming the induced pluripotent stem cell. In another embodiment, the induced pluripotent stem cell is prepared by a process including the steps of transfecting a cord blood stem cell with a nucleic acid encoding an OCT4 protein to form a transfected cord blood stem cell. The transfected cord blood stem cell is allowed to divide thereby forming the induced pluripotent stem cell.

### EXAMPLES

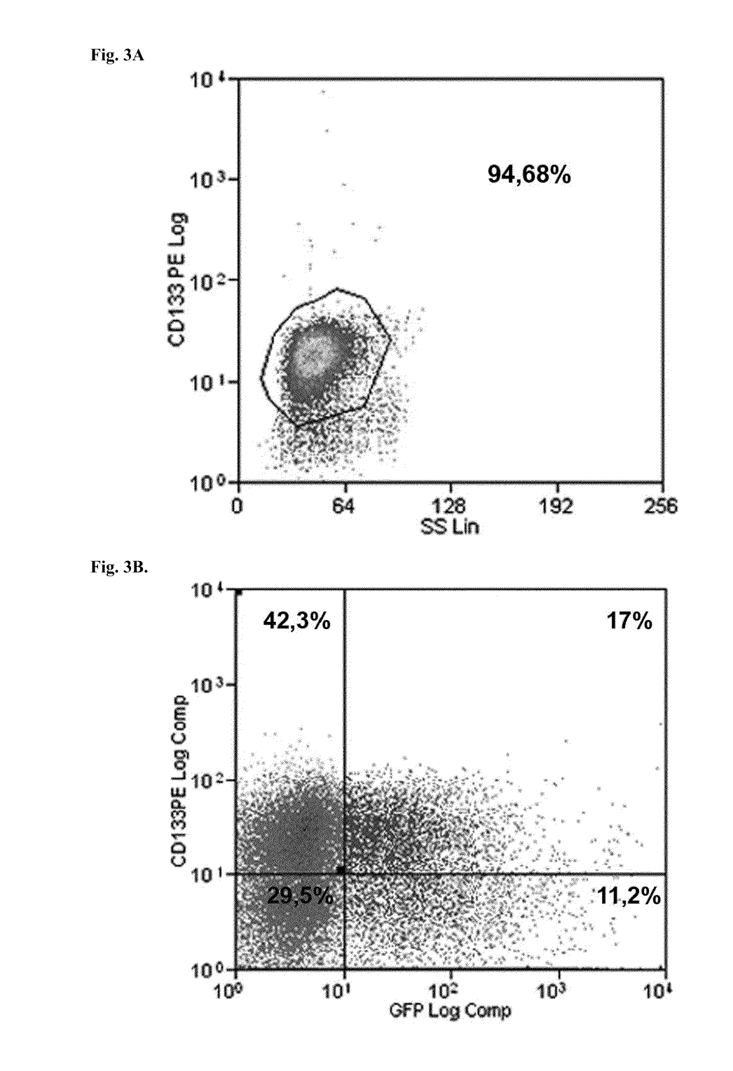

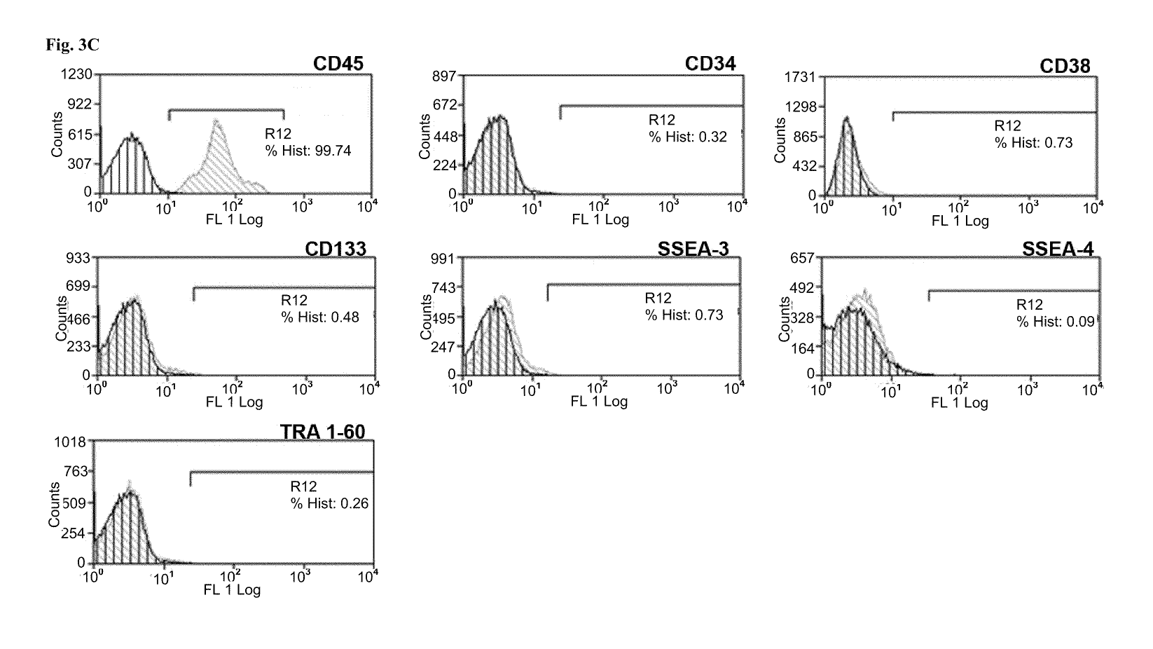

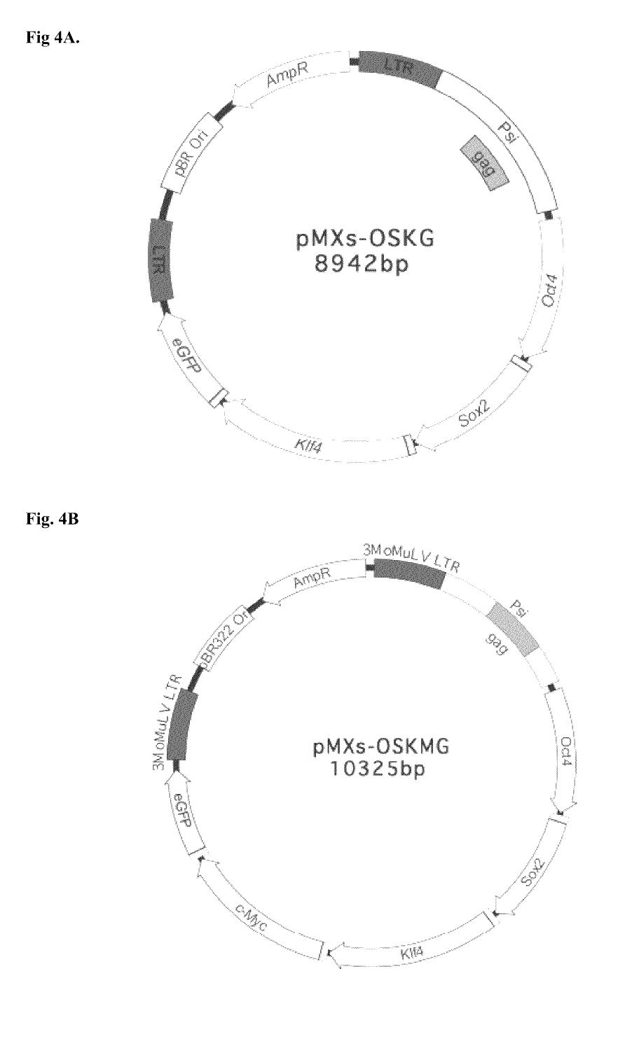

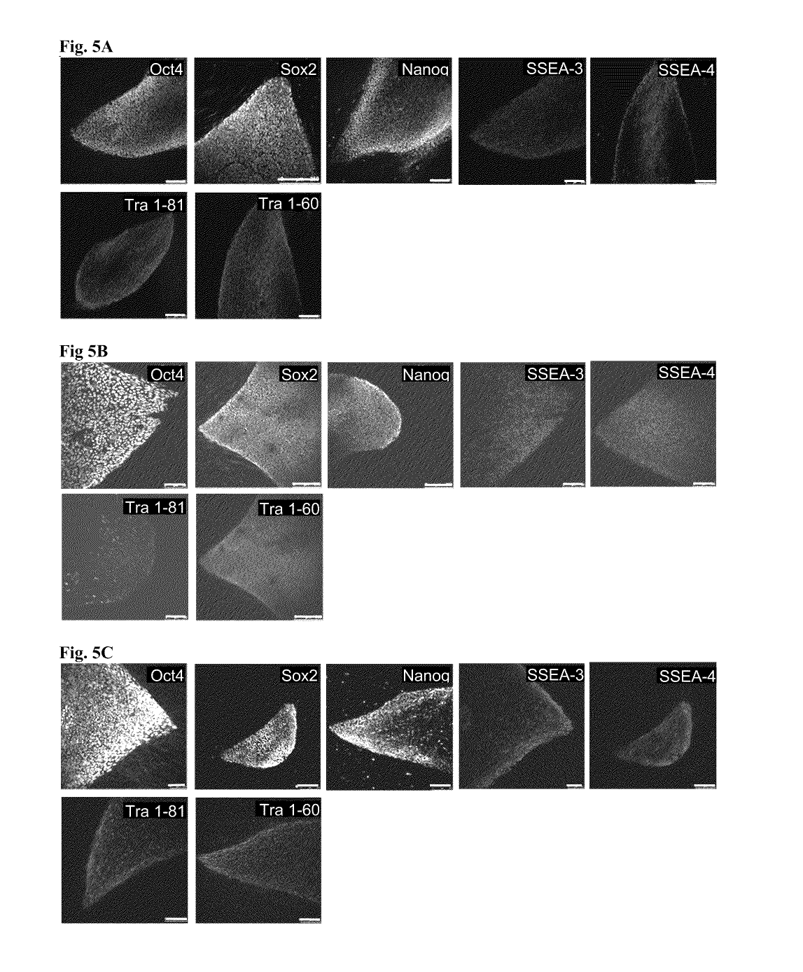

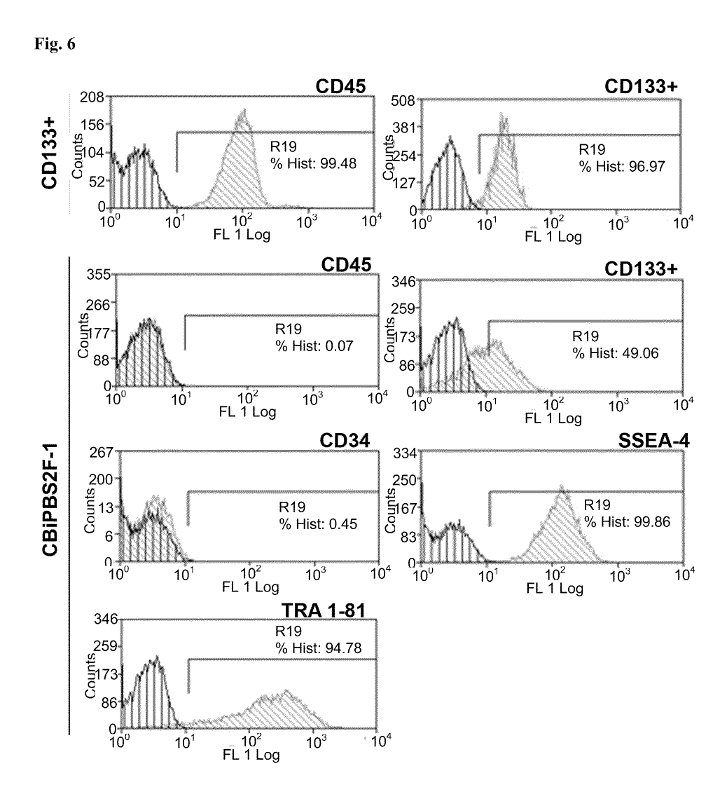

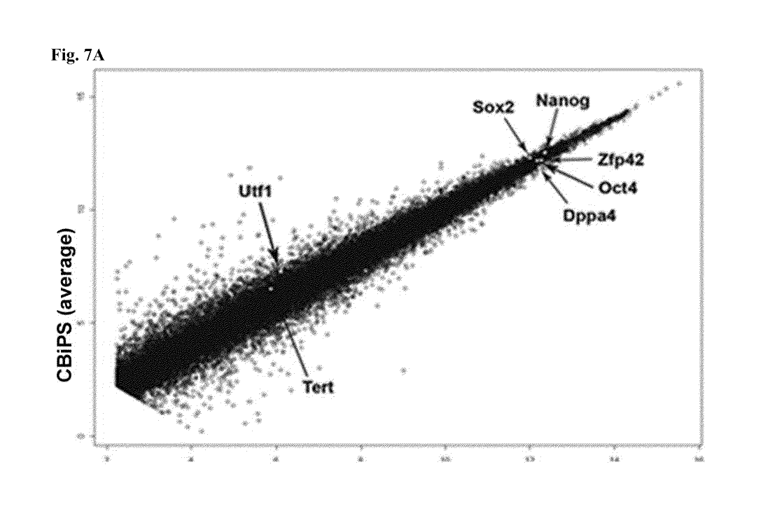

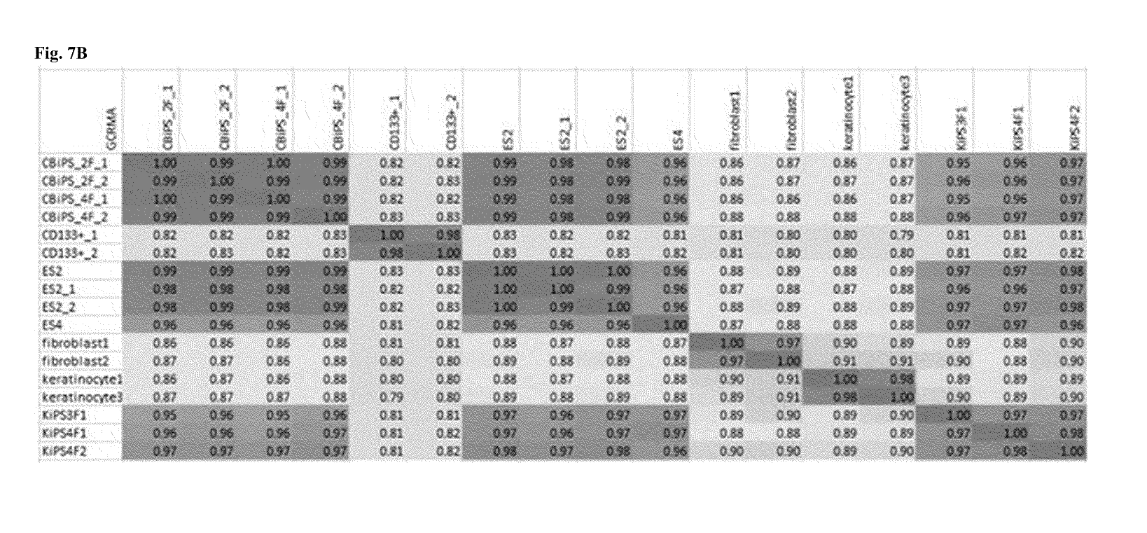

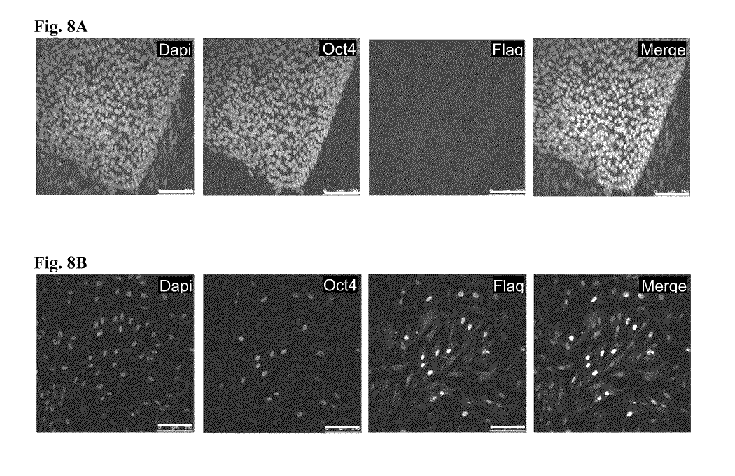

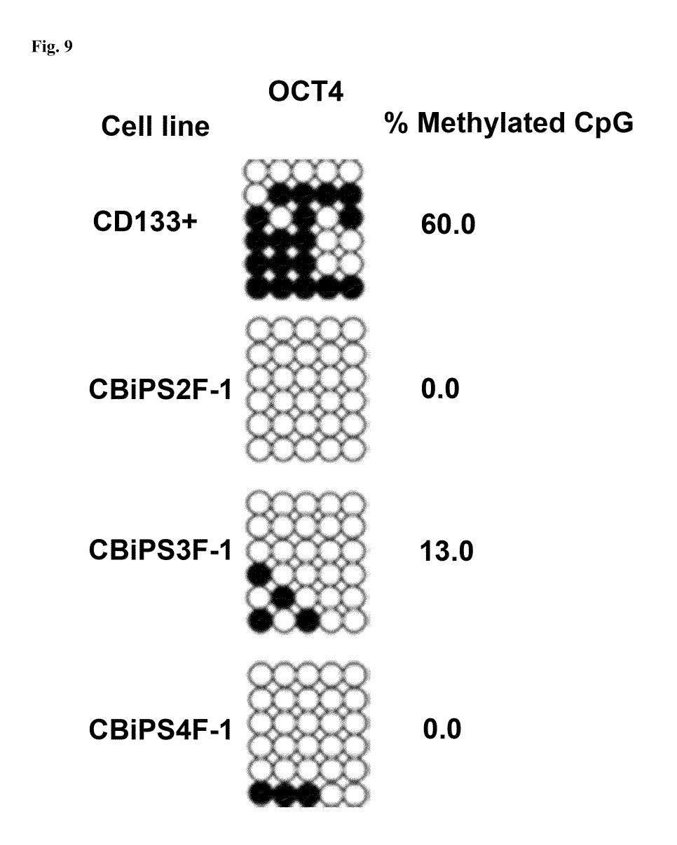

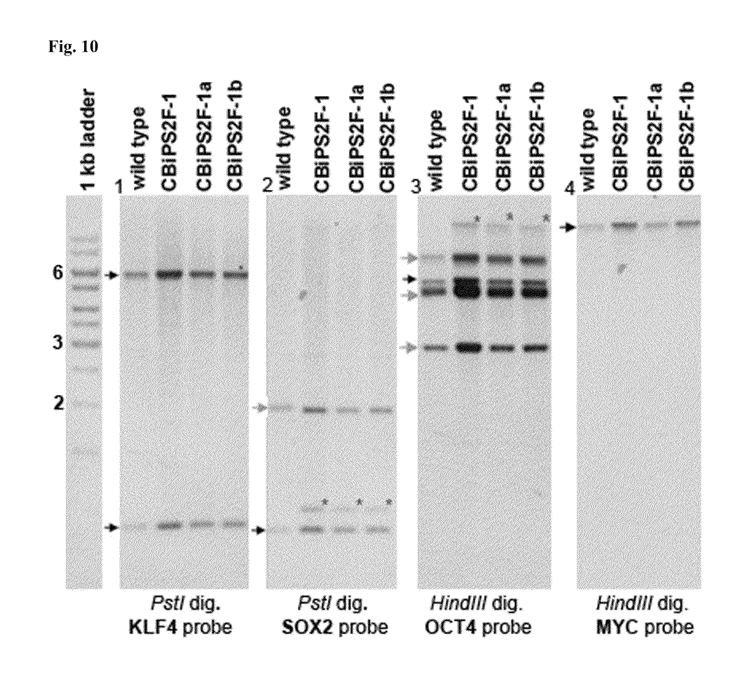

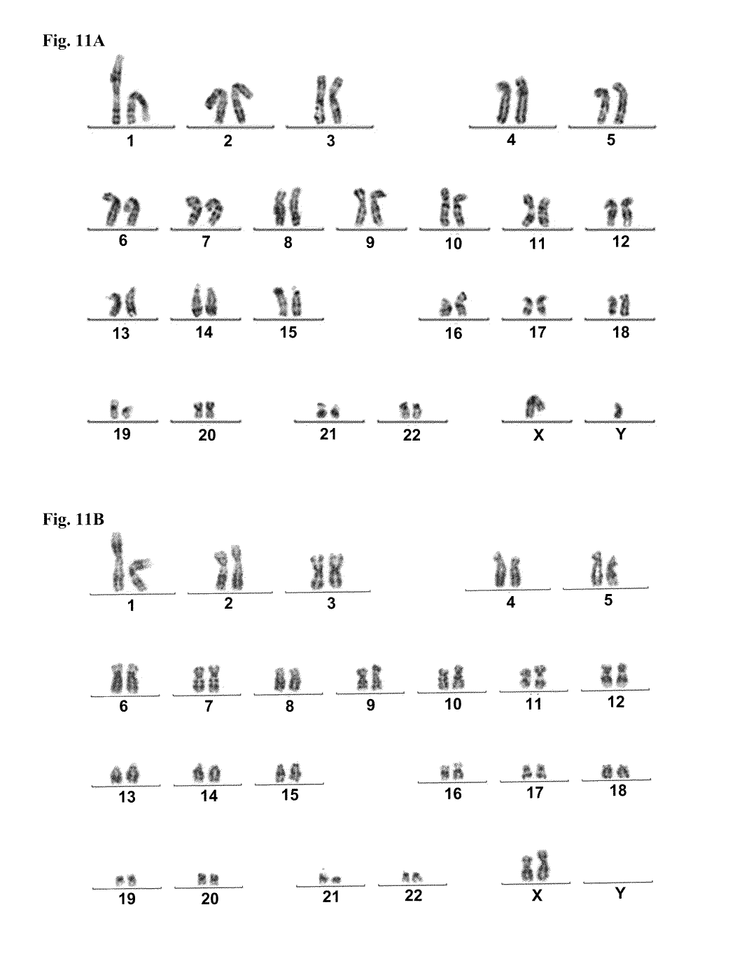

To isolate CB stem cells, CD133+ cells were purified from CB units, using immuno-magnetic selection, obtaining a population of cells with a purity range of 90-94% (FIG. 3A). In order to promote the proliferation of the quiescent CD133+ stem cells, cells were cultured in presence of Stem Cell Factor (SCF), Trombopoietin (TPO), Flt ligand 3 (Flt3) and Interleukin 6 (IL-6) for 24 hours. These cells were infected following a protocol for adherent cells,¹⁰ with some modifications. Briefly, cells were seeded over retronectin-coated plates previously pre-adsorbed with the viral particles as previously described.¹⁹ In control experiments, the purified CD133+ population was subjected to three cycles of infection every 12 h using a constitutive GFP retrovirus and the resulting population were analyzed three days post-infection using flow cytometry; FIG. 3B shows a typical experimental outcome: 28% of the total cells were GFP positive and 58% were still positive for the CD133 antigen. Within the GFP positive population, 17% were CD133+/GFP+, while 11% were CD133−/GFP+.

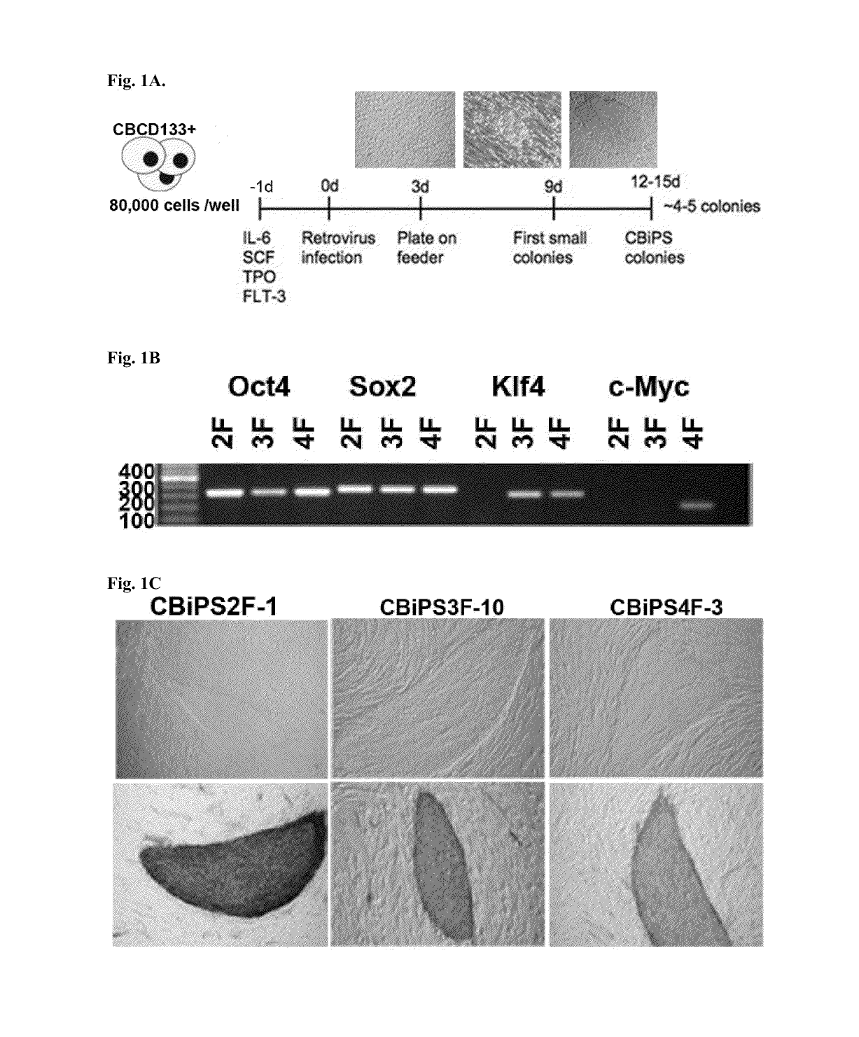

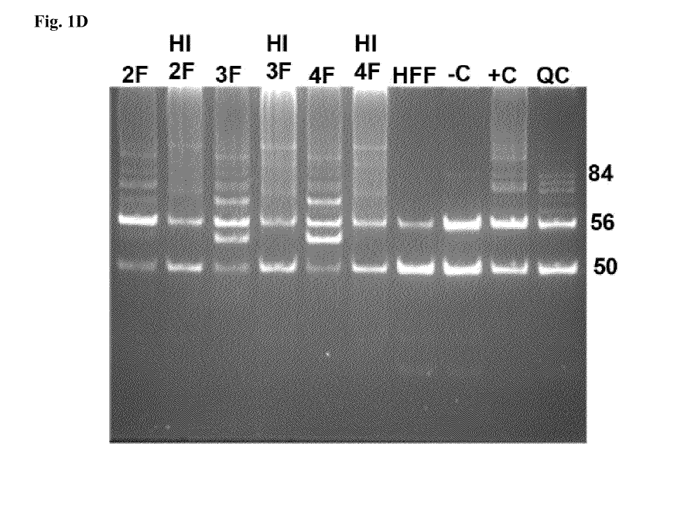

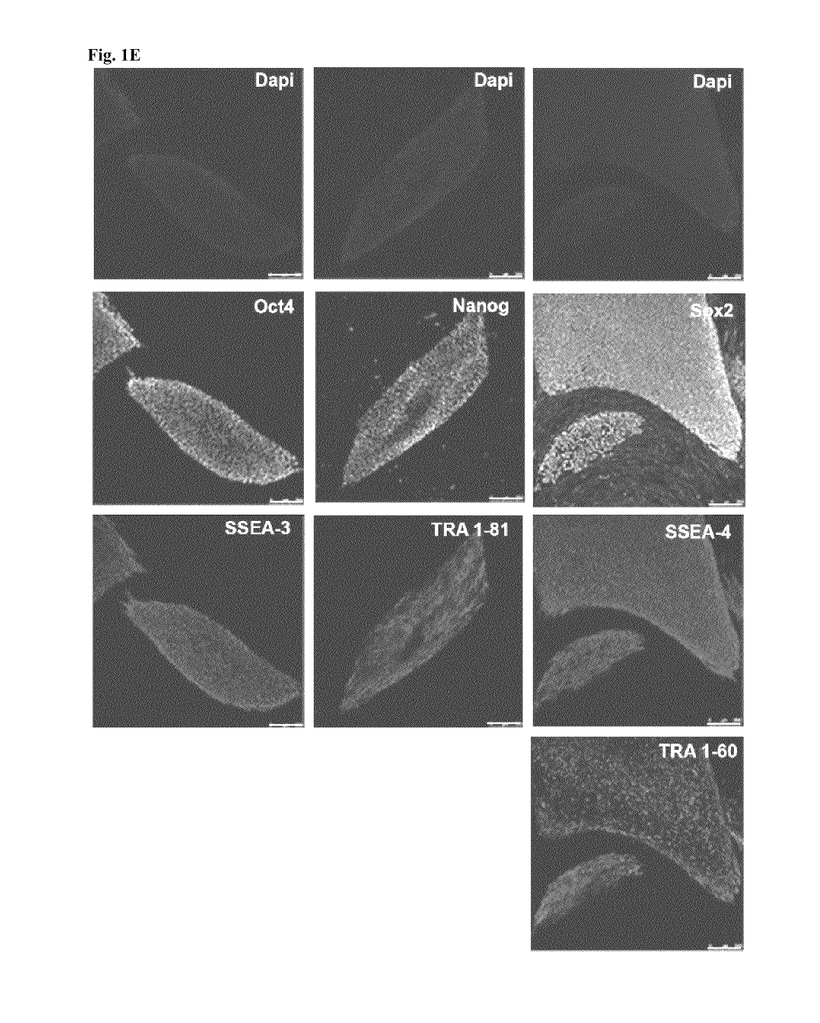

It was attempted to reprogram CD133+ cells using any single or combination of the OSKM factors. Three days post transduction, cells were plated onto irradiated Human Foreskin Fibroblasts (HFF-1) feeder cells and cultured in hES medium with bFGF. To rule out the possibility that mere ES-like culture conditions could induce reprogramming per se, untransduced CD133+ stem cells were also cultured for 3 weeks in hES cell conditions. No colony formation was observed in these plates. Flow cytometry analysis of those cells revealed that they no longer expressed the stem cells markers CD133, CD34, and CD38, remained positive for the haematopoietic marker CD45, but did not acquire the embryonic markers SSEA-3, SSEA-4 or TRA1-60, altogether suggesting that untransduced CD133+ cells differentiate into mature haematopoietic cells when cultured in hES cell conditions (FIG. 1C).

Around 9 days post infection, small colonies started to appear in cells that had been transduced with OSKM, OSK or OS. At 12 to 15 days post infection, some of the colonies exhibited typical hES cell morphology, with sharp borders, and were comprised of a small, tightly packed cell population with large nuclei and clearly visible nucleoli (FIG. 1A). From a standard infection of 8×10⁴ CD133+ cells, 4 to 5 hES-like colonies were observed and were named CBiPS. The colonies were expanded by manual picking and a CBiPS line from each factor combination (CBiPS 4F-1, CBiPS 3F-1, CBiPS 2F-1) was amplified for further characterization. The presence of each retroviral transgene was confirmed by PCR genotyping, demonstrating the insertion of the expected 4, 3 or 2 transcription factors in CBiPS 4F-1, CBiPS 3F-1, CBiPS 2F-1, respectively (FIG. 1B).

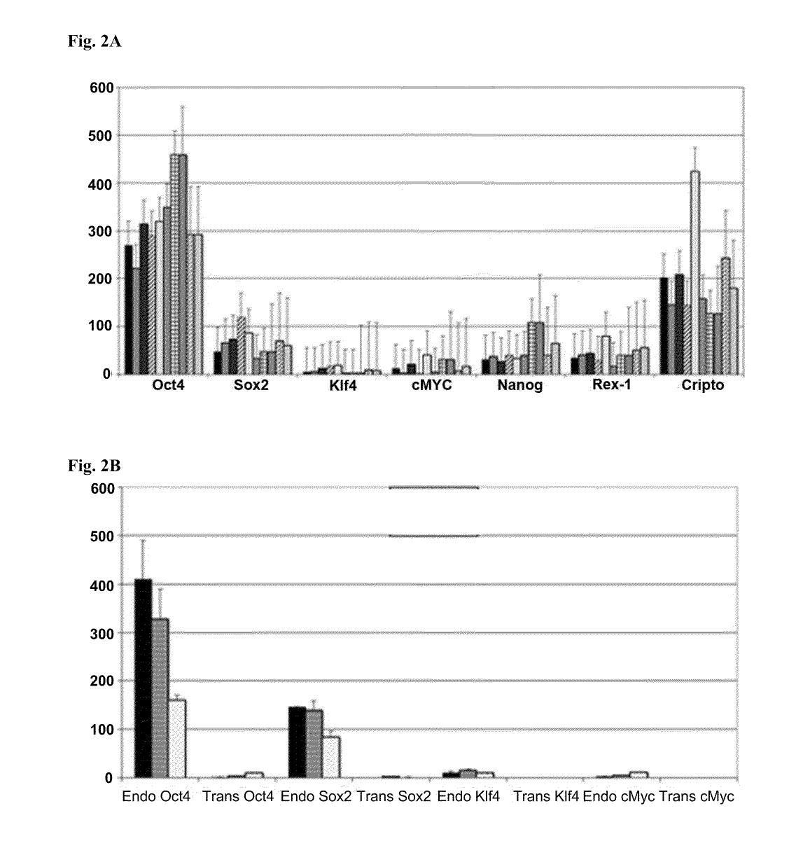

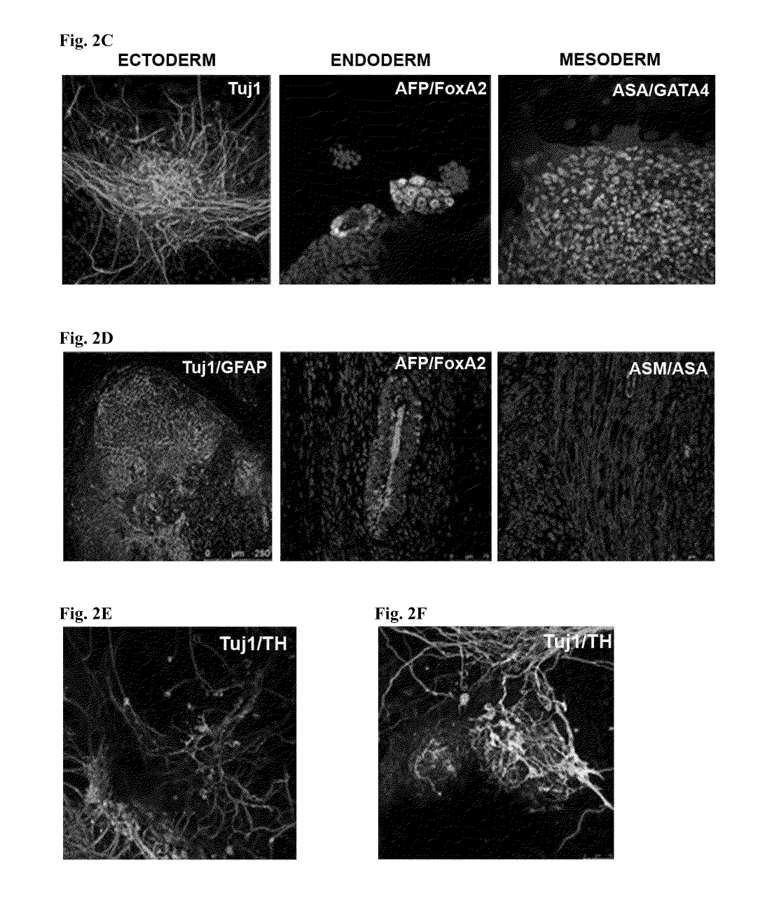

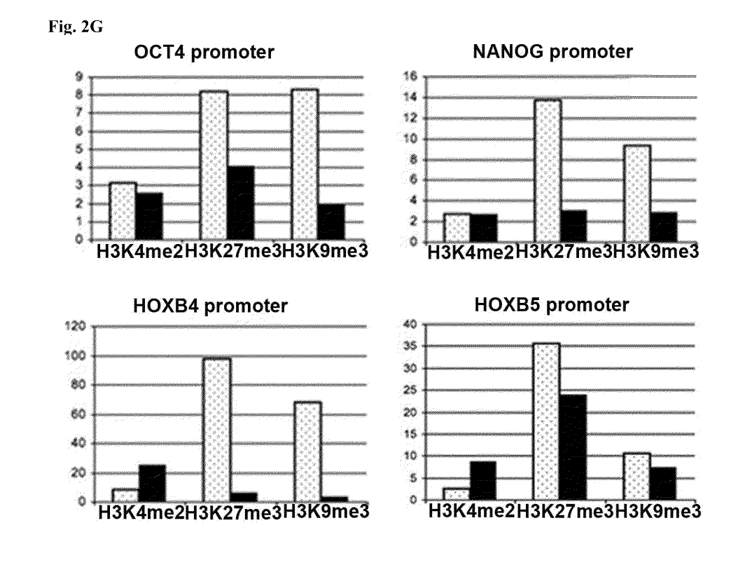

All three CBiPS lines stained positive for Alkaline Phosphatase (FIG. 1C) and expressed the pluripotency markers OCT4, SOX2, TRA-1-81, TRA-1-60, SSEA3, SSEA4, and NANOG, as assessed by immunofluorescence staining (FIG. 1D). In addition, reprogrammed CBiPS lines were negative for the haematopoietic stem cell markers CD45, CD34, and CD38 as assayed by flow cytometry. They were, however, positive for CD133, a common marker of haematopoietic and embryonic stem cells (FIG. 1E). Consistent with the previous immuno-characterization, Real Time PCR analysis showed that all three CBiPS lines expressed a number of pluripotency genes including OCT4, SOX2, NANOG, CRIPTO and REX, uncovering a gene-expression profile comparable to other iPS¹⁰ and hES [2] cell lines²⁰ (FIG. 2A). In addition, the expression of the retroviral trangenes was properly silenced, and expression of OCT4, SOX2, KLF4 and c-MYC in CBiPS lines was driven by the corresponding endogenous genes (FIG. 2B). This was also confirmed by immunofluorescence staining using antibodies specific for FLAG-tagged transgenic factors (FIGS. 8A-8B).

CD133+ stem cells, but not fibroblasts and keratinocytes, expressed low levels of endogenous OCT4, NANOG, SOX2, REX1 and CRIPTO (FIG. 2C) thus pointing towards a more plastic epigenetic state allowing rapid reprogramming. Moreover, it was also found that the promoters of OCT4 and NANOG in CD133+ cells have lower levels of histone repressive marks (H3K7 and H3K9 methylation) compared to fibroblasts (FIG. 2D), suggesting the presence of a more permissive chromatin organization that might favour the binding and the transcriptional activation of these genes by the overexpressed factors. In addition, the combination of high levels of KLF4 and c-MYC in CB CD133+ stem cells compared to fibroblasts and keratinocytes (FIG. 2C, FIG. 2E) might further indicate that endogenous expression of these factors may allow a more rapid and/or an enhanced reprogramming of those cells.¹⁰

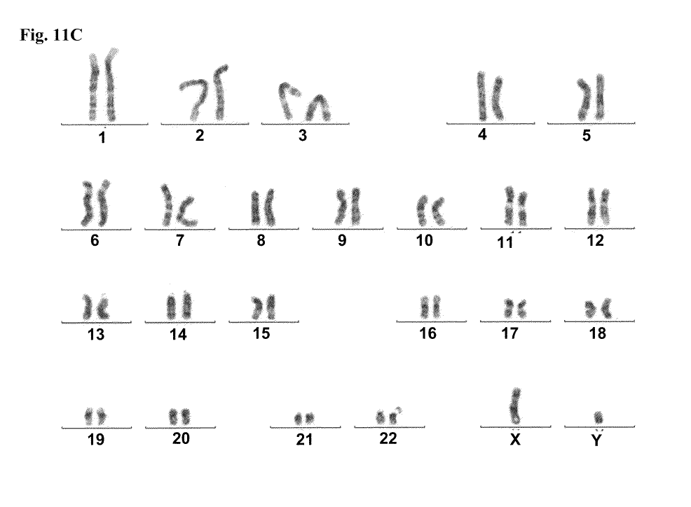

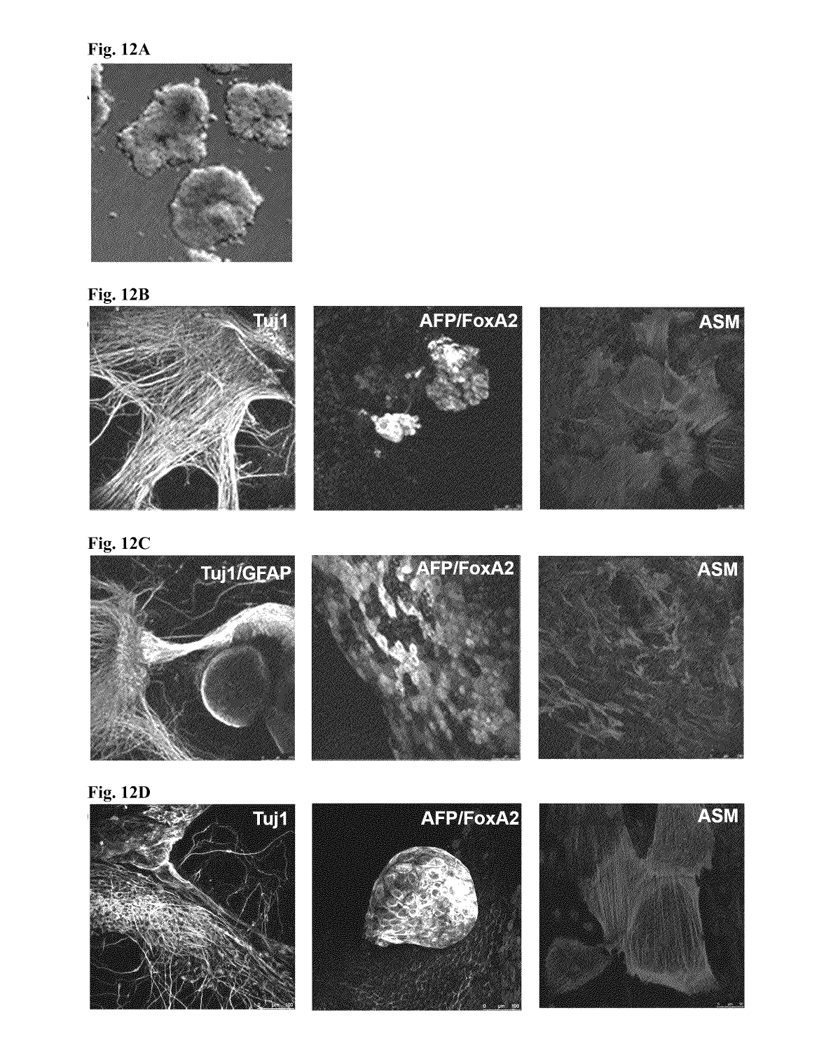

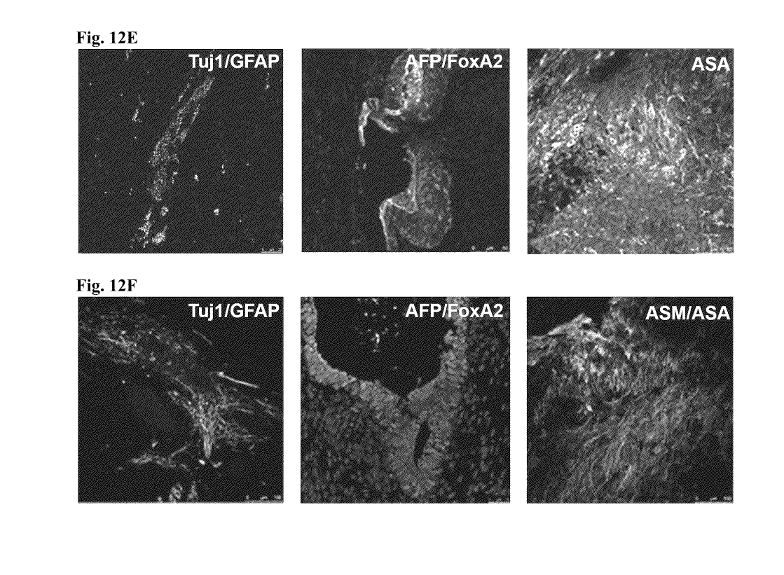

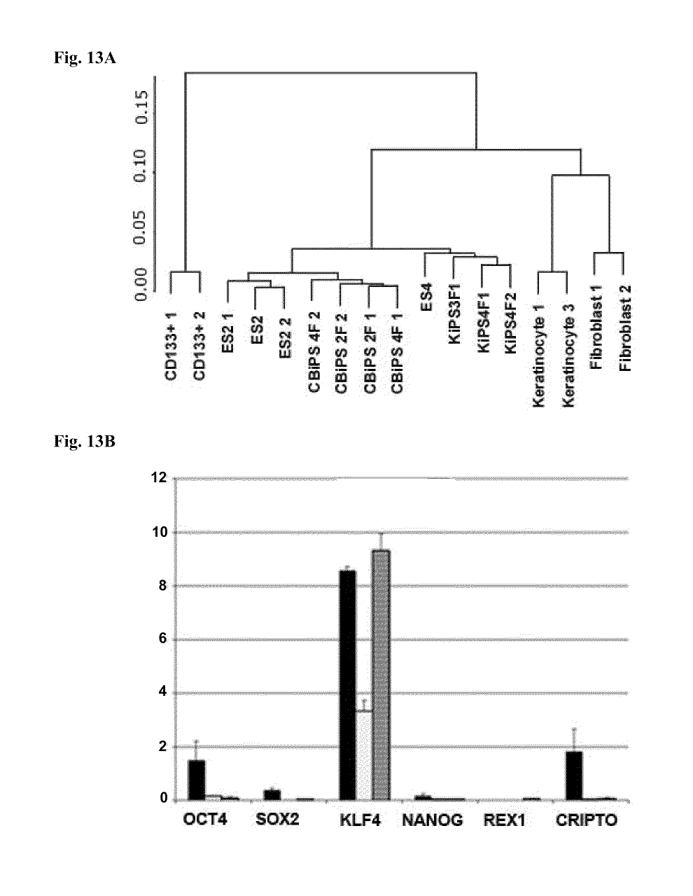

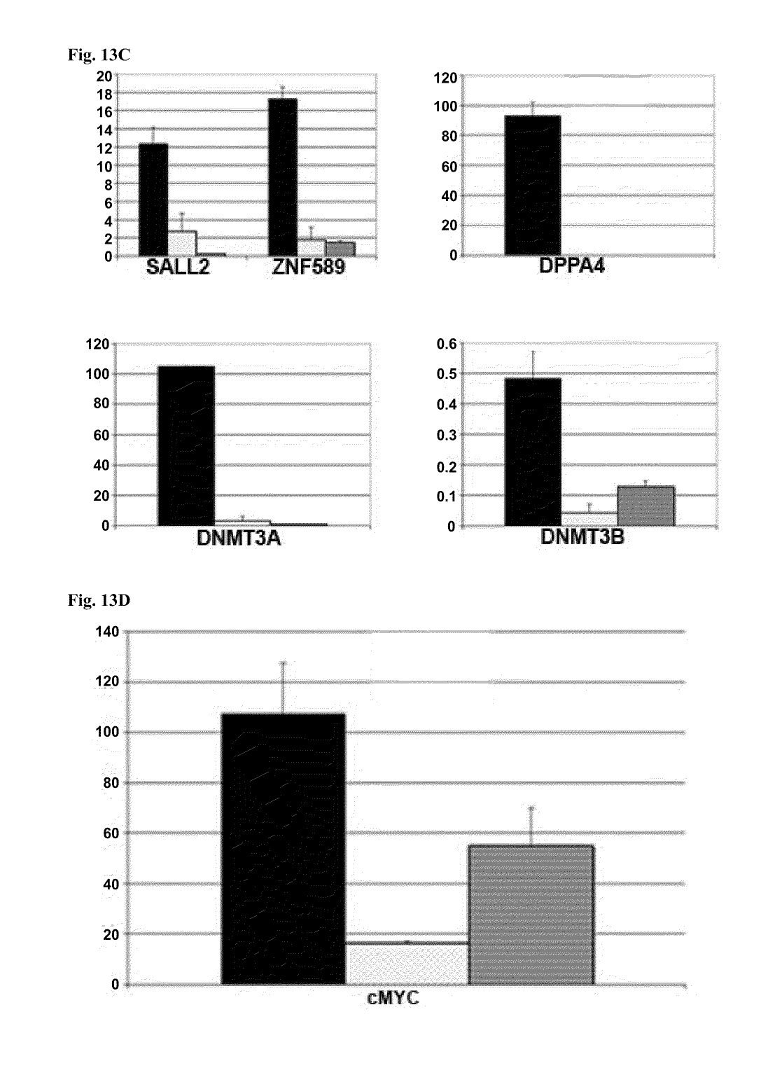

Cytogenetic analysis showed that the three cell lines maintain a normal 46XY karyotype after 10 passages. In addition, the male chromosomal content excludes the possibility that the reprogrammed cells arise from a small fraction of contaminating mother cells known to be present in the initial cord blood sample (FIG. 11A). Next the differentiation potential of CBiPS cell lines by in vitro embryoid body formation was evaluated. All cell lines were able to form embryoid bodies (EBs) with high efficiency (FIG. 12A), and the EBs could be differentiated into cell types of all three embryonic germ layers, including FoxA2 and α-actinin positive mesoderm, GFAP and Tuj1 positive ectoderm, and α-fetoprotein positive endoderm (FIGS. 12B-12F). The results confirm that the CBiPS 4F-1, CBiPS 3F-1, CBiPS 2F-1 cell lines are transcriptionally reprogrammed to a similar state as other hiPS and hES cell lines, are karyotypically stable, and display in vitro developmental potentialities consistent with pluripotency.

Reprogramming of somatic cells has been accomplished through expression of a combination of pluripotency factors and oncogenes. The fact that it was possible to reprogram CB CD133+ stem cells in only two weeks and using two factors highlights the potential of CB cells as an ideal source of somatic cells towards developing clinically suitable iPS cells for regenerative medicine. This may include the use of non-integrative or semi-integrative approaches,²¹⁻²³ as well as the replacement of OCT4 or SOX2 by small molecules.^{24,25} As far as other amenable somatic cell sources are concerned, it was recently shown that mobilized peripheral blood (mPB) cells could also represent an effective source for iPS derivation.²⁶ However, compared to newborn CB stem cells, adult mPB cells are more likely to accumulate genomic alterations as a result of ageing or as a direct consequence of a specific disease. Moreover, the pharmacological treatment used to mobilize the adult haematopoietic stem cell compartment represents a health risk for the donor: in a small but sizeable fraction of donors, this procedure can induce severe reactions, including splenic rupture.²⁷

CB stem cells overcome these problems, are easily accessible and, due to their early origin, are still immunologically immature, allowing for less stringent criteria for HLA-donor-recipient selection.¹⁵ To date, more than 400,000 CB units are available worldwide in a comprehensive network of cord blood banks, facilitating a rapid and effective search for compatible donors for CBiPS generation². Even though the generation of patient specific iPS lines has been repeatedly advocated as a theoretically ideal clinical option, from a practical and cost-benefit aspect, this approach may be in many cases unfeasible. Large scale production and banking of CBiPS lines representing a variety of HLA haplotypes in a publicly available network would represent an invaluable tool for basic research and future clinical applications of human iPS cells.

### VI. Materials and Methods

### Sample Collection

Umbilical CB samples were obtained from the Banc de Sang i Teixits, Hospital Duran i Reynals, Barcelona.

### CD133+ Cell Purification

Mononuclear cells (MNC) were isolated from CB using Lympholyte-H (Cederlane, Ontario, Calif.) density gradient centrifugation. CD133+ cells were positively selected using Mini-Macs immunomagnetic separation system (Miltenyi Biotec, Bergisch Gladbach, Germany). Purification efficiency was verified by flow cytometric analysis staining with CD133-phycoerythrin (PE; Miltenyi Biotec, Bergisch Gladbach, Germany) antibody.

### Constructs and Retroviral Production

OCT4 and SOX2 human cDNAs were amplified from ES[4] total RNA by RT-PCR; human KLF4 was amplified from IMAGE clone 5111134 and the mutant human c-MYCT58A was amplified from a DNA template kindly provided by Luciano Di Croce. The amplified cDNAs were cloned into the EcoRI/ClaI sites of a modified pMSCVpuro vector that allows the expression of N-terminal FLAG-tagged proteins. pMXs-OSKMG was constructed as follows: the mouse Oct4 cDNA was amplified using a reverse primer eliminating the Oct4 stop codon and adding a BspEI site and cloned into pCRII (Invitrogen) to give pCRII-Oct4-Bsp (oriented NotI-5′ cDNA3′-Acc651). The mouse Sox2 cDNA was amplified using a forward primer containing an AgeI site followed by P2A peptide sequence and a reverse primer eliminating the Sox2 stop codon and containing a BspEI site; this fragment was cloned in pCRII to give pCRII-Age-Sox2-Bsp (oriented NotI-5′ cDNA3′-Acc65I). pCRII-Age-Sox2-Bsp was cut AgeI and Acc65I and cloned into pCRII-Oct4-Bsp cut BspEI-Acc65I producing pCRII-Oct4-P2A-Sox2-BspEI. The same cloning approach was repeated twice in order to incorporate mouse Klf4 and eGFP (producing pCRII-OSKG) or mouse Klf4, c-Myc and eGFP (producing pCRII-OSKMG). Finally pCRII-OSKG and pCRII-OSKMG were cut EcoRI and cloned into a unique EcoRI site of the retroviral empty vector pMXs, producing pMXs-OSKG and pMXs-OSKMG. Retroviruses for the four factors were independently produced after transfecting the cell line Phoenix Amphotropic using Fugene 6 reagent (Roche) according to manufacturer's directions. After 24 hours, the medium was replaced, cells were incubated at 32° C., and viral supernatant was harvested every 12 hours.

### Transduction of CD133+ Cells

CB CD133+ cells (1×10⁵ cells per ml) were pre-stimulated for 24 h in DMEM supplemented with 10% of FBS in the presence of SCF (50 ng/ml)+Flt3 (50 ng/ml)+TPO (10 ng/ml)+IL-6 (10 ng/ml)(PeproTech). Multi-well non-tissue culture-treated plates were coated with retronectin (Takara, Otsu, Japan, www.takara-bio.com), a fibronectin fragment CH-296 (15 mg/cm²), and preloaded by centrifuging the plates with a filtered 1:1:1:1 mix of retroviral supernatant for OCT4, SOX2, KLF4, and c-MYC factors at the 2,500 RPM for 30 minutes. About 80,000 CD133+ cells were plated in the presence of DMEM+10% FBS and the cytokine cocktail mentioned above. Every 12 h, half of the medium was replaced with fresh viral supernatant containing the cytokine cocktail and incubated at 37° C., 5% CO₂; three infection cycles were performed. At day 3, the cells were harvested and transferred into 6 well-plates containing irradiated human fibroblasts and ES medium, consisting of KO-DMEM medium (Invitrogen) supplemented with 20% KO-Serum Replacement (GIBCO), non-essential amino acids (Lonza), 2-β-mercaptoethanol (GIBCO), Penicillin/Streptomycin (GIBCO), GlutaMAX™ (Invitrogene), and 10 ng/ml bFGF (Peprotech). CBiPS cells were cultured on top of irradiated human fibroblasts and picked mechanically.

### Purification of Total RNA and Quantitative RT-PCR

Isolation of total RNA from CB CD133+ stem cells, hES[2] cells, KiPS cells (14) and CBiPS was performed using either Trizol reagent (Invitrogen, Carlsbad, Calif.) or RNAqueous®-Micro kit (Ambion Inc., Austin Tex.) based on the cell number available. All samples were treated with TURBO DNase inhibitor (Ambion) to remove any residual genomic DNA and 1 ug of RNA was used to synthesize cDNA using the Invitrogen SuperScript™ II Reverse Transcriptase kit. 25 ng of cDNA were used to quantify gene expression by Quantitative RT-PCR using primers as previously described.¹°

### Genechip® Expression Analysis

The GeneChip® microarray processing was performed by the Functional Genomica Core in the Institute for Research in Biomedicine (Barcelona, Spain) according to the manufacturer's protocols (Affymetrix, Santa Clara, Calif.). The amplification and labelling were processed as indicated in Nugen protocol with 25 ng starting RNA. For each sample, 3.75 μg ssDNA were labelled and hybridized to the Affymetrix HG-U133 Plus 2.0 chips. Expression signals were scanned on an Affymetrix GeneChip Scanner (7G upgrade). The data extraction was done by the Affymetrix GCOS software v.1.4. The statistical analysis of the data was performed using the program R from the R Project for Statistical Computing. First, the raw data was normalized using the gcRMA algorithm implemented in R, and a hierarchical clustering using Pearson correlation coefficients was performed on the normalized data. To integrate datasets obtained for two different experiments (our keratinocyte reprogramming (GEO accession number: GSE12583), and this experiment (GSE16694), we normalized together the raw CEL files using the gcRMA algorithm in R, and then corrected the batch effect using the ComBat algorithm, as known in the art. See e.g., Johnson et al., 2007, Biostatistics 8:118-127.

### Southern Blot

Genomic DNA from each cell line was isolated using All Prep DNA/RNA columns (Qiagen), following manufacturer's guidelines. Each lane of the Southern blot corresponds to 4 ug of genomic DNA digested with 40 U of either PstI or HindIII restriction enzyme (New England Biolabs), electrophoreses on a 1% agarose gel, transferred to a neutral nylon membranes (Hybond™-N, Amersham) and hybridized with DIG-dUTP labeled probes generated by PCR using the PCR DIG Probe Synthesis Kit (Roche Diagnostics). Probes were detected by an AP-conjugated DIG-Antibody (Roche Diagnostics) using CDP-Star (Sigma-Aldrich) as a substrate for chemiluminescence. Conditions were as per the instructions of the manufacturer. The probes were generated using SOX2, OCT4, KLF4 and c-MYC cDNAs as templates with the following primers (F, forward; R, reverse):

| (SEQ ID NO: 10) | |
|---|---|
| SOX2 F | 5′-AGTACAACTCCATGACCAGC-3′; |
| (SEQ ID NO: 11) | |
| SOX2 R | 5′-TCACATGTGTGAGAGGGGC-3′; |
| (SEQ ID NO: 12) | |
| OCT4 F | 5′-TAAGCTTCCAAGGCCCTCC-3′; |
| (SEQ ID NO: 13) | |
| OCT4 R | 5′-CTCCTCCGGGTTTTGCTCC-3′; |
| (SEQ ID NO: 14) | |
| KLF4 F | 5′-AATTACCCATCCTTCCTGCC-3′; |
| (SEQ ID NO: 15) | |
| KLF4 R | 5′-TTAAAAATGCCTCTTCATGTGTA-3′; |
| (SEQ ID NO: 16) | |
| c-MYC F | 5′-TCCACTCGGAAGGACTATCC-3′; |
| (SEQ ID NO: 17) | |
| c-MYC R | 5′-TTACGCACAAGAGTTCCGTAG-3. |

### Immunofluoresence Analysis and AP Analyses.

CBiPS were grown on plastic coverslide chambers and fixed with 4% paraformaldehyde (PFA). The following antibodies were used: TRA-1-60 (MAB4360, 1:200), TRA-1-81 (MAB4381, 1:200), SOX2 (AB5603, 1:500) all Chemicon, SSEA-4 (MC-813-70, 1:2), SSEA-3 (MC-631, 1:2) all Iowa, Tuj1 (1:500; Covance), α-fetoprotein (1:400; Dako), α-actinin (1:100; Sigma), OCT4 (C-10, SantaCruz, sc-5279, 1:100), NANOG (Everest Biotech EB06860, 1:100), GATA 4 (1:50, SantaCruz), smooth muscle actin (1:400, Sigma), FoxA2 (1:50 R&D System), GFAP (1:1000, Dako), α-sarcomeric actin (1:400, Sigma), Anti-Flag (Sigma M2). Images were taken using a Leica SP5 confocal microscope. Direct AP activity was analysed using an Alkaline Phosphatase Blue/Red Membrane substrate solution kit (Sigma) according to the manufacturer's guidelines.

### In Vitro Differentiation

EBs formation was induced from colony fragments mechanically collected and then maintained in suspension in presence of hES medium for 24 hours. We performed a pre condition culture for 2-3 days where the EBs were maintained in the three different differentiation medium onto ultra-low attachment plates. In particular, for endoderm differentiation the EBs were cultured in the presence of KO-DMEM medium supplemented with 10% fetal bovine serum (FBS), 2 mM L-glutamine, 0.1 mM 2-β-mercaptoethanol, non-essential amino acids, and penicillin-streptomycin. For mesoderm differentiation, we used the same medium described above, but adding ascorbic acid (0.5 mM). For ectoderm induction, the EBs were cultured in N2/B27 medium. After the precondition step, the EBs in endoderm and mesoderm conditions were transferred to 0.1% gelatine-coated plastic chamber slides and cultured in differentiation medium and differentiation medium plus acid ascorbic (0.5 nM) respectively, for 2 weeks. For the ectoderm differentiation, the EBs were transferred onto stromal cell line PA6 and in the presence of N2/B27 medium for 2 weeks. The medium for each condition was changed every other day.

### Chromatin Immunoprecipitation

Chromatin immunoprecipitation experiments were performed using the Magnetic Low cell ChIP Kit from Diagenode following the manufacturer's instructions and using 15,000 cells per immunoprecipitation. Antibodies used were from Millipore 07-440 (anti-H3K27me3), 07-030 (anti-H3K4me2) and 17-625 (anti-H3K9me3).

### Promoter Methylation Analysis

Genomic DNA was extracted by samples of about 500.000 CD133+ and CBiPS cells using QIA AMP DNA Mini Kit (Qiagen). Two micrograms of purified DNA was mutagenised with Epitect Bisulfite Kit (Qiagen) according to manufacturer specifications. The promoter sequences of interest were amplified by two subsequent PCRs using primers previously described.¹⁰ The resulting amplified products were cloned into pGEM T Easy plasmids, amplified in TOP10 cells, purified and sequenced.

### Teratoma Formation

Severe combined immunodeficient (SCID) beige mice (Charles River Laboratories) were anesthetized and approximately 0.5×10⁶ CBiPS cells, resuspended in 20-40 μl of hES media, were injected into the testis. Mice were euthanized 6-8 weeks after cell injection and tumours were processed and analyzed following conventional immunohistochemistry protocols (Masson's trichromic stain) and immunofluorescence.

### VII. References

1. Yamanaka, S. A fresh look at iPS cells. Cell 137, 13-7 (2009).
2. Gluckman, E. & Rocha, V. Cord blood transplantation: state of the art. Haematologica 94, 451-4 (2009).
3. Takahashi, K. & Yamanaka, S. Induction of pluripotent stem cells from mouse embryonic and adult fibroblast cultures by defined factors. Cell 126, 663-76 (2006).
4. Takahashi, K. et al. Induction of pluripotent stem cells from adult human fibroblasts by defined factors. Cell 131, 861-72 (2007).
5. Takahashi, K., Okita, K., Nakagawa, M. & Yamanaka, S. Induction of pluripotent stem cells from fibroblast cultures. Nat Protoc 2, 3081-9 (2007).
6. Yu, J. et al. Induced pluripotent stem cell lines derived from human somatic cells. Science 318, 1917-20 (2007).
7. Park, I. H. et al. Reprogramming of human somatic cells to pluripotency with defined factors. Nature 451, 141-6 (2008).
8. Hochedlinger, K. & Plath, K. Epigenetic reprogramming and induced pluripotency. Development 136, 509-23 (2009).
9. Lowry, W. E. et al. Generation of human induced pluripotent stem cells from dermal fibroblasts. Proc Natl Acad Sci USA 105, 2883-8 (2008).
10. Aasen, T. et al. Efficient and rapid generation of induced pluripotent stem cells from human keratinocytes. Nat Biotechnol 26, 1276-84 (2008).
11. Silva, J. et al. Promotion of reprogramming to ground state pluripotency by signal inhibition. PLoS Biol 6, e253 (2008).
12. Eminli, S., Utikal, J., Arnold, K., Jaenisch, R. & Hochedlinger, K. Reprogramming of neural progenitor cells into induced pluripotent stem cells in the absence of exogenous Sox2 expression. Stem Cells 26, 2467-74 (2008).
13. Kim, J. B. et al. Oct4-induced pluripotency in adult neural stem cells. Cell 136, 411-9 (2009).
14. Kim, J. B. et al. Pluripotent stem cells induced from adult neural stem cells by reprogramming with two factors. Nature 454, 646-50 (2008).
15. Rocha, V. et al. Transplants of umbilical-cord blood or bone marrow from unrelated donors in adults with acute leukemia. N Engl J Med 351, 2276-85 (2004).
16. Kucia, M. et al. Morphological and molecular characterization of novel population of CXCR4+SSEA-4+ Oct-4+ very small embryonic-like cells purified from human cord blood: preliminary report. Leukemia 21, 297-303 (2007).
17. Nikolova, T. et al. WNT-conditioned media differentially affect the proliferation and differentiation of cord blood-derived CD133+ cells in vitro. Differentiation 75, 100-11 (2007).
18. Zhao, Y., Wang, H. & Mazzone, T. Identification of stem cells from human umbilical cord blood with embryonic and hematopoietic characteristics. Exp Cell Res 312, 2454-64 (2006).
19. Gammaitoni, L. et al. Serial transplantations in nonobese diabetic/severe combined immunodeficiency mice of transduced human CD34+ cord blood cells: efficient oncoretroviral gene transfer and ex vivo expansion under serum-free conditions. Stem Cells 24, 1201-12 (2006).
20. Raya, A. et al. Generation of Cardiomyocytes from New Human Embryonic Stem Cell Lines Derived from Poor-quality Blastocysts. Cold Spring Harb Symp Quant Biol (2008).
21. Yu, J. et al. Human Induced Pluripotent Stem Cells Free of Vector and Transgene Sequences. Science (2009).
22. Woltjen, K. et al. piggyBac transposition reprograms fibroblasts to induced pluripotent stem cells. Nature 458, 766-70 (2009).
23. Kaji, K. et al. Virus-free induction of pluripotency and subsequent excision of reprogramming factors. Nature 458, 771-5 (2009).
24. Huangfu, D. et al. Induction of pluripotent stem cells from primary human fibroblasts with only Oct4 and Sox2. Nat Biotechnol 26, 1269-75 (2008).
25. Shi, Y. et al. A combined chemical and genetic approach for the generation of induced pluripotent stem cells. Cell Stem Cell 2, 525-8 (2008).
26. Loh, Y. H. et al. Generation of induced pluripotent stem cells from human blood. Blood (2009).
27. Anderlini, P. Effects and safety of granulocyte colony-stimulating factor in healthy volunteers. Curr Opin Hematol 16, 35-40 (2009).

What is claimed is:

1. A method for preparing an induced pluripotent stem cell comprising:
(i) transfecting a cord blood stem cell with a nucleic acid encoding an OCT4 protein and a nucleic acid encoding a SOX2 protein to form a transfected cord blood stem cell, and
(ii) allowing said transfected cord blood stem cell to divide thereby forming said induced pluripotent stem cell.

2. The method of claim 1, wherein said cord blood stem cell is not transfected with an additional nucleic acid encoding a cMYC protein, a LIN28 protein, a NANOG protein or a KLF4 protein.

3. A method for preparing an induced pluripotent stem cell comprising:
(i) transfecting a cord blood stem cell with a nucleic acid encoding an OCT4 protein to form a transfected cord blood stem cell, and
(ii) allowing said transfected cord blood stem cell to divide thereby forming said induced pluripotent stem cell.

4. The method of claim 3, wherein said cord blood stem cell is not transfected with an additional nucleic acid encoding a cMYC protein, a LIN28 protein, a NANOG protein or a KLF4 protein.

5. The method of claim 1, wherein said cord blood stem cell expresses a CD133 antigen.

6. The method of claim 1, wherein said cord blood stem cell is derived from fresh cord blood.

7. The method of claim 1, wherein said cord blood stem cell is derived from frozen cord blood.

8. An induced pluripotent stem cell prepared in accordance with the method of claim 1.

9. A cord blood stem cell comprising a nucleic acid encoding an OCT4 protein and a nucleic acid encoding a SOX2 protein.

10. The cord blood stem cell of claim 9, wherein said cord blood stem cell consists essentially of a nucleic acid encoding an OCT4 protein and a nucleic acid encoding a SOX2 protein.

11. A cord blood stem cell comprising a nucleic acid encoding an OCT4 protein.

12. The cord blood stem cell of claim 11, wherein said cord blood stem cell consists essentially of a nucleic acid encoding an OCT4 protein.

13. The cord blood stem cell of claim 9, wherein said cord blood stem cell expresses a CD133 antigen.

14. The cord blood stem cell of claim 9, wherein said cord blood stem cell is derived from fresh cord blood.

15. The cord blood stem cell of claim 9, wherein said cord blood stem cell is derived from frozen cord blood.

16. A method for producing a human somatic cell comprising:
(i) contacting an induced pluripotent stem cell with cellular growth factors; and
(ii) allowing said induced pluripotent stem cell to divide, thereby forming said human somatic cell;
wherein said induced pluripotent stem cell is prepared by a process comprising the steps of:
(i) transfecting a cord blood stem cell with a nucleic acid encoding an OCT4 protein and a nucleic acid encoding a SOX2 protein to form a transfected cord blood sem cell, and
(ii) allowing said transfected cord blood stem cell to divide thereby forming said induced pluripotent stem cell;
or wherein said induced pluripotent stem cell is prepared by a process comprising the steps of:
(i) transfecting a cord blood stem cell with a nucleic acid encoding an OCT4 protein to form a transfected cord blood stem cell, and
(ii) allowing said transfected cord blood stem cell to divide thereby forming said induced pluripotent stem cell.

17. A method of treating a mammal in need of tissue repair comprising:
(i) administering an induced pluripotent stem to said mammal,
(ii) allowing said induced pluripotent stem cell to divide and differentiate into somatic cells in said mammal, thereby providing tissue repair in said mammal;
wherein said induced pluripotent stem cell is prepared by a process comprising the steps of:
(i) transfecting a cord blood stem cell with a nucleic acid encoding an OCT4 protein and a nucleic acid encoding a SOX2 protein to form a transfected cord blood stem cell, and
(ii) allowing said transfected cord blood stem cell to divide thereby forming said induced pluripotent stem cell;
or wherein said induced pluripotent stem cell is prepared by a process comprising the steps of:
(i) transfecting a cord blood stem cell with a nucleic acid encoding an OCT4 protein to form a transfected cord blood stem cell, and
(ii) allowing said transfected cord blood stem cell to divide thereby forming said induced pluripotent stem cell.
